(19) United States
(12) Patent Application Publication
(10) Pub. No.: US 2025/0243243 A1
(43) Pub. Date: Jul. 31, 2025

(54) DISRUPTION OF SONIC HEDGEHOG-SURF4 INTERACTION FOR CANCER TREATMENT

(71) Applicant: The Hong Kong University of Science and Technology, Hong Kong (CN)

(72) Inventors: Yusong GUO, Hong Kong (CN); Xiao TANG, Hong Kong (CN); Tingxuan WANG, Hong Kong (CN)

(21) Appl. No.: 18/833,263

(22) PCT Filed: Feb. 10, 2023
(86) PCT No.: PCT/IB2023/000075

§ 371 Date: Jul. 25, 2024

Related U.S. Application Data
(60) Provisional application No. 63/309,648, filed on Feb. 14, 2022.

(51) Int. Cl. C07K 7/08; A61K 31/727; A61K 31/737; A61K 38/00; A61P 35/00; C07K 7/06

(52) U.S. Cl. CPC C07K 7/08; A61K 31/727; A61K 31/737; A61P 35/00; C07K 7/06; A61K 38/00

(57) ABSTRACT

Provided are compositions and methods that block the interaction between Sonic hedgehog (Shh) and Surfeit locus protein 4 (SURF4), particularly for use in treating subjects with cancer or at risk of suffering from cancer. The subject methods block the interaction between SURF4 and Shh, either by mutating residue E50, D53, D56, or any combination thereof of SURF4 or mutating the CW motif (amino acid residues 32-38) on human Shh. Also provided is a composition comprising a polypeptide that contains the CW motif at the position of 32-38 on human Shh, a polypeptide that contains the first luminal loop (residues 49-60) of human SURF4, or small chemical molecules that block the interaction between SURF4 and Shh and administering said composition to a subject, particularly in subjects with cancer or at risk of suffering from cancer.

### CROSS-REFERENCE TO RELATED APPLICATION

This application claims the benefit of U.S. Provisional Application Ser. No. 63/309,648, filed Feb. 14, 2022, which is hereby incorporated by reference in its entirety including any tables, figures, or drawings.

### BACKGROUND OF THE INVENTION

The Hedgehog (Hh) signaling pathway plays an important role in various developmental processes in metazoans (1, 2). Mutations of key components that regulate Hh signaling are associated with many human diseases (3). Hh was first found in the Drosophila larval epidermis. It mediates larval segment development and adult appendage patterning (4). In mammals, there are three Hh family members, Sonic hedgehog (Shh), Indian hedgehog (Ihh) and Desert hedgehog (Dhh). Ihh regulates the proliferation and differentiation of chondrocytes (5). Dhh functions in gonads, regulating testis organogenesis, spermatogenesis (6, 7), and follicle development in the ovary (8). Shh functions more extensively than the other two Hh members: it regulates embryonic patterning (4), specification of cell types in the nervous system (9), axon guidance (10), cell differentiation and organ development (11).

Hh is synthesized as a full-length precursor Hh (HhFL). After entering the ER, HhFL is auto-cleaved into two parts: an N-terminal Hedge domain (HhN) and a C-terminal Hog domain (HhC) (1). HhC is degraded through ER-associated degradation (12). HhN undergoes lipid modifications, in which a cholesterol molecule is covalently linked to the C-terminus and a palmitoyl group is linked to the N-terminus (13-15). Lipid-modified HhN subsequently exits the ER and is delivered via the secretory pathway to the plasma membrane. Once at the plasma membrane, Hh is released into the extracellular matrix (ECM) and ultimately recognized by its receptors on the plasma membrane of target cells to induce downstream signal transduction.

Although significant progress has been achieved in understanding the Hh signaling pathway in target cells, the molecular mechanisms that mediate secretion of newly synthesized Shh proteins from the producing cells are still unclear. The ER is the first station where newly synthesized proteins enter the secretory pathway. In this compartment, cargo proteins are generally recognized by the coat protein complex II (COPII) to be packaged into vesicles and exported from the ER. Soluble cargo proteins in the ER lumen cannot directly engage the COPII coat, but instead are captured into vesicles by transmembrane cargo receptors. One mammalian cargo receptor, ERGIC53, is a mannose-specific lectin that recognizes N-linked glycoproteins in the ER lumen (16, 17). The p24 family of proteins function as cargo receptors to regulate ER export of glycosylphosphatidylinositol (GPI)-anchored proteins (18). Mammalian orthologs of yeast ER vesicle (Erv) proteins have also been thought to function as cargo receptors (16). Surfeit locus protein 4 (SURF4), the mammalian ortholog of Erv29p, regulates ER export of soluble proteins, including lipoproteins and proprotein convertase subtilisin/kexin type 9 (PCSK9) (19-21). SURF4 recognizes amino-terminal tripeptide motifs of soluble cargo proteins, and participates in ER exit site (ERES) organization (19, 22). The cargo receptors that mediate sorting of Shh in the secretory pathway remain unknown.

Abnormal activation of the Hh signaling pathway has been shown to promote cancer progression. All current Hh pathway inhibitors approved by FDA or undergoing clinical trials target components downstream of Hh ligand reception, such as SMO and Gli. However, these inhibitors failed to show efficacy in ligand-dependent cancer types.

Therefore, there remains a need to target Shh signaling to inhibit cancer progression, particularly ligand-dependent cancer types.

### BRIEF SUMMARY OF THE INVENTION

The subject invention pertains to compositions and methods that block the interaction between Sonic hedgehog (Shh) and Surfeit locus protein 4 (SURF4), particularly for use in treating subjects with cancer or at risk of suffering from cancer. The subject methods block the interaction between SURF4 and Shh, either by mutating residue E50, D53, D56, or any combination thereof of SURF4 or mutating the Cardin-Weintraub (CW) motif (amino acid residues 32-38) on human Shh. Amino acid residues 32-38 in human Shh are corresponding to amino acid residues 33-39 in mouse Shh. The invention further pertains to compositions comprising a polypeptide that contains the CW motif at the position of 32-38 on human Shh, a polypeptide that contains the first luminal loop (residues 49-60) of human SURF4, small chemical molecules that block the interaction between SURF4 and Shh, or any combination thereof and to methods of administering the compositions comprising a polypeptide that contains the CW motif at the position of 32-38 on human Shh, a polypeptide that contains the first luminal loop (residues 49-60) of human SURF4, small chemical molecules that block the interaction between SURF4 and Shh, or any combination thereof to a subject in need thereof.

In certain embodiments, the small chemical molecule is a glycosaminoglycan, such as, for example, heparin or heparin sulfate.

In certain embodiments, the CW motif in Shh is necessary and sufficient for ER-to-Golgi transport of ShhN. Surfeit locus protein 4 (SURF4) interacts directly with the CW motif of ShhN to regulate packaging of ShhN into COPII vesicles. ShhN and SURF4 interact with each other at the ER and separate from each other after entering the Golgi. The CW motif is known to interact with proteoglycans (PGs) that are predominantly synthesized at the Golgi. PGs compete with SURF4 to bind ShhN, and that inhibiting synthesis of PGs causes defects in export of ShhN from the trans Golgi network (TGN). SURF4 and PG maturation are also important for intracellular traffic of full length Shh in mammalian cells.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIGS. 1A-1N. ER-to-Golgi transport of ShhN depends on its KRRHPKK (SEQ ID NO: 6) motif. FIG. 1A and FIG. 1D. HeLa cells were transfected with plasmids encoding Str-KDEL and SBP-EGFP-ShhN²⁵⁻¹⁹⁸ (FIG. 1A Panels A-B, FIG. 1D Panels K-M) or SBP-EGFP tagged fragments or a mutant version of ShhN (FIG. 1A Panels C-H, FIG. 1D Panels N-P). Day 1 after transfection, the localization of the different versions of RUSH constructs containing ShhN was analyzed after incubation with biotin for the indicated time. Scale bar, 10 μm. Magnification, 63×. FIG. 1B and FIG. 1E. Quantifications of the percentage of cells showing juxta-nuclear-accumulated EGFP signal after incubation with biotin for the indicated time (mean±S.D.; n=3; >100 cells counted for each time point). **, p<0.01. FIG. 1C. Sequence alignment of amino acids 33-49 of mouse Shh across species. FIG. 1F. HEK293T cells were transfected with plasmids encoding the indicated constructs. On day 1 after transfection, cells were incubated with biotin for 2 hr. After biotin incubation, the level of wild type (wt) or mutant versions of SBP-EGFP-ShhN²⁵⁻¹⁹⁸-HA in the culture medium and in cell lysates was analyzed by immunoblotting using anti-HA and anti-SEC22B antibodies. FIG. 1G. Quantification of the level of secreted SBP-EGFP-ShhN²⁵⁻¹⁹⁸-HA normalized to that detected in the wt group (mean±S.D.; n=3). In each experimental group, the secreted abundance of ShhN after biotin treatment is normalized to the abundance of ShhN in cell lysates before biotin treatment. **, p<0.01. FIG. 1H. Diagram depicting the vesicle formation assay to reconstitute release of ShhN-HA into transport vesicles. FIGS. 11-1K and FIG. 1N. Vesicle formation was performed using the reagents as indicated in cells transfected with plasmids encoding wt or mutant versions of ShhN¹⁻¹⁹⁸-HA. The vesicle fraction was analyzed by immunoblotting using anti-HA or anti-ERGIC53 antibodies. FIGS. 1L-1M. Quantification of the percentage of ShhN-HA (FIG. 1L) or ERGIC53 (FIG. 1M) that was packaged into transport vesicles (mean±S.D.; n=3). ***, p<0.001; N.S., not significant.

FIGS. 2A-2K. SURF4 mediates packaging of ShhN into transport vesicles and regulates ER-to-Golgi trafficking and the secretion of ShhN. FIG. 2A. HeLa cells were transfected with negative control (NC) siRNA or two different siRNAs against SURF4. 24 hr after transfection, cells were re-transfected with plasmids encoding SBP-EGFP-ShhN²⁵⁻¹⁹⁸ and Str-KDEL. On day 3 after knockdown, cells were incubated with biotin for the indicated time and the localization of the indicated proteins was analyzed using antibodies against endogenous TGN46 and SURF4. Scale bar, 10 μm. Magnification, 63×. FIGS. 2C-2D. The level of SURF4 and ERGIC53 in cell lysates from Hela cells transfected with NC siRNA or with siRNA against SURF4 (FIG. 2C) and from HEK293Trex WT or SURF4 KO cells (FIG. 2D) were analyzed by immunoblotting with anti-SURF4 and anit-ERGIC53 antibodies. FIG. 2E. Wild type or SURF4 KO HEK293Trex cells were transfected with plasmids encoding SBP-EGFP-ShhN²⁵⁻¹⁹⁸ and Str-KDEL. On day 3 after knockdown, cells were incubated with biotin for the indicated time and the localization of ShhN was analyzed. Scale bar, 10 μm. Magnification, 63×. FIG. 2B and FIG. 2F. Quantifications of the percentage of cells showing juxta-nuclear-localized SBP-EGFP-ShhN²⁵⁻¹⁹⁸ (mean±S.D.; n=3; >100 cells counted for each experiment). **, p<0.01; ***, p<0.001; ****, p<0.0001. FIG. 2G. Wild type or SURF4 KO cells were transfected with the indicated amount of plasmids encoding HA-tagged ShhN¹⁻¹⁹⁸ (ShhN-HA). Day 1 after transfection, the levels of ShhN-HA in the medium and in cell lysates were analyzed by immunoprecipitation and immunoblotting with anti-HA antibodies. FIGS. 2H-2I. Vesicle formation was performed using the indicated reagents in wild type cells (FIG. 2H) and SURF4 KO cells (FIG. 2I). The vesicle fraction was analyzed by immunoblotting using anti-ERGIC53 or anti-HA antibodies. FIGS. 2J-2K. Quantification of the percentage of ShhN-HA (FIG. 2J) or ERGIC53 (FIG. 2K) that was packaged into transport vesicles (n=3, mean±S.D.). **, p<0.01; N.S., not significant.

FIGS. 3A-3O. The CW motif of ShhN directly interacts with the predicted first luminal loop of SURF4. FIG. 3A. Purified GST or GST-tagged human ShhN²⁵⁻⁴⁹ was incubated with lysates from HEK293T cells transfected with SURF4-HA. After incubation, the bound proteins were analyzed by immunoblotting with anti-HA antibodies. FIG. 3B. Relative levels of SURF4-HA that bound to GST or ShhN²⁵⁻⁴⁹-GST were quantified (n=3, mean±S.D.). The level of SURF4-HA that bound to GST or ShhN²⁵⁻⁴⁹-GST was normalized to the corresponding bait protein, and this value was then normalized to the level of SURF4-HA that bound to GST in each experimental group. ****, p<0.0001. FIG. 3C. HEK293T cells were co-transfected with plasmids encoding the indicated constructs. Day 1 after transfection, cells were treated in 2 mM DSP, and cell lysates were incubated with beads conjugated with HA antibodies. The bound proteins were analyzed by immunoblotting with anti-HA or anti-Myc antibodies. FIG. 3D. The percentage of SURF4 that bound to ShhN^{⊗25-49}-HA was normalized to that bound to ShhN-HA. The normalized abundance was then quantified (n=3, mean±S.D.). ***, p<0.001. FIG. 3E. The structure of SURF4 predicted by AlphaFold. Hydrophobic amino acids are highlighted in light blue. FIG. 3F. Co-IP was performed in HEK293T cells expressing the indicated constructs in the presence of DSP. The bound proteins were analyzed by immunoblotting with anti-HA or anti-Myc antibodies. FIG. 3G. The percentage of SURF4^{WT}-Myc that bound to ShhN-HA was normalized to the percentage of SURF4^{ED-AA}-Myc. The normalized abundance was then quantified (n=3, mean±S.D.). *, p<0.05. FIG. 3H and FIG. 3J. Purified GST, Shh²⁵⁻⁴⁹-GST, GST-SURF4⁴⁹⁻⁶⁰ were purified from E.coli and analyzed by Coomassie blue staining. FIG. 3I and FIG. 3K. CW or SURF4-lumenal peptides were covalently linked to thiopyridone sepharose 6B, and incubated with the indicated prey proteins. After incubation, the bound proteins were analyzed by immunoblotting with anti-GST antibodies. FIGS. 3L-3M. Isothermal titration calorimetry (ITC)-based measurement of the interaction between CW peptides and GST-SURF4⁴⁹⁻⁶⁰ or GST. FIG. 3N. CW, CW(KR-AA), or RRFR peptides were covalently linked to thiopyridone sepharose 6B, incubated with GST-SURF4⁴⁹⁻⁶⁰. After incubation, the bound proteins were analyzed by immunoblotting with anti-GST antibodies. FIG. 3O. Levels of GST-SURF4⁴⁹⁻⁶⁰ bound to the indicated peptides were quantified (n=3, mean±S.D.). The quantification is normalized to the level of GST-SURF4⁴⁹⁻⁶⁰ that bound to CW peptides in each experimental group. **, p<0.01; ****, p<0.0001.

FIGS. 4A-4E Synthesis of proteoglycans regulates export of ShhN out of the TGN. FIG. 4A. HeLa cells were treated (or not) with 2.5 mM xyloside. 24 hr after xyloside treatment, cells were transfected with plasmids encoding Str-KDEL and SBP-EGFP-ShhN²⁵⁻¹⁹⁸. 48 hr after xyloside treatment, cells were treated with biotin and incubated at 20° C. for 2 hr. Then the cells were incubated at 32° C. for 0 min (FIG. 4A Panels A-F) or 45 min (FIG. 4A Panels G-L). The localizations of SBP-EGFP-ShhN²⁵⁻¹⁹⁸ and TGN46 were analyzed. Scale bar, 10 μm. Magnification, 63×. The magnified views of the indicated areas in panels A, D, G, J are shown in panels A′, D′, G′, J′. FIG. 4B. Quantification of the number of punctate structures labelled with SBP-EGFP-ShhN²⁵⁻¹⁹⁸ in each expressing cell (n=3, mean±S.D., over 25 cells were quantified in each experimental group). ****, p<0.0001. FIG. 4C. HeLa cells were transfected with NC siRNA or two different siRNAs against XYLT2. 48 hr after transfection, cells were re-transfected with plasmids encoding Str-KDEL and SBP-EGFP-ShhN²⁵⁻¹⁹⁸. On day 3 after knockdown, cells were treated with biotin and incubated in 20° C. for 2 hr. Then the cells were incubated at 32° C. for 0 or 45 min and the localization of Shh was analyzed. Scale bar, 10 μm. Magnification, 63×. The magnified views of the indicated area in panels Q-S are shown in panels Q′-S′. FIG. 4D. HEK293T cells were transfected with negative control (NC) siRNA or siRNA against XYLT2. 48 h after transfection, cells were re-transfected with plasmids encoding Myc-XYLT2. On day 3 after knockdown, the level of SEC22B and Myc-XYLT2 in cell lysates was analyzed by immunoblotting using anti-Myc or anti-SEC22B antibodies. FIG. 4E. Quantifications of the number of punctate structures containing SBP-EGFP-ShhN²⁵⁻¹⁹⁸ per cell at different time points after biotin treatment (n=3, mean±S.D., over 20 cells were quantified in each experimental group). **, p<0.01.

FIGS. 5A-5H. PGs compete with SURF4 to bind ShhN and facilitate the trafficking of ShhN through the Golgi. FIG. 5A. Purified GST-tagged human ShhN²⁵⁻⁴⁹ was incubated with lysates from HEK293T cells transfected with SURF4-HA in the presence of the indicated concentrations of heparin. After incubation, the bound proteins were analyzed by immunoblotting with anti-HA antibodies. FIG. 5B. Relative levels of SURF4-HA that bound to ShhN²⁵⁻⁴⁹-GST were quantified (n=3, mean±S.D.). The quantification is normalized to the level of SURF4-HA that is bound to ShhN²⁵⁻⁴⁹-GST in the absence of heparin in each experimental group. *, p<0.05. FIGS. 5C-5D. HEK293T cells were transfected with negative control (NC) siRNA or siRNAs against XYLT2. 48 hr after transfection, cells were re-transfected with plasmids encoding SURF4-Myc and SBP-EGFP-ShhN²⁵⁻¹⁹⁸-HA (referred to as SBP-EGFP-ShhN-HA). On day 3 after knockdown, cells were incubated at 20° C. for 2 hr in the absence or presence of biotin. Then cells were treated with 2 mM DSP, and the cell lysates were incubated with beads conjugated with HA antibodies. The bound proteins were analyzed by immunoblotting with anti-HA or anti-Myc antibodies. FIG. 5E. Relative levels of SURF4-Myc that bound to SBP-EGFP-ShhN-HA were quantified (n=3, mean±S.D.). In each experimental group, the levels of bound SURF4-Myc after biotin treatment are normalized to the levels of bound SURF4-Myc before biotin treatment.*, p<0.05. FIG. 5F. Our model depicting the molecular mechanisms regulating sorting and secretion of ShhN. FIG. 5G. HeLa cells transfected with negative control siRNA (NC) or siRNA against XYLT2 (XYLT2 KD) were treated with biotin for 20 or 30 min, and the localizations of the indicated proteins were analyzed. Scale bar, 10 μm. Magnification, 63×. The magnified views of the indicated area in panels L, P, S, V, Y were shown in panels L′, P′, S′, V′, Y′. FIG. 5H. Quantifications of the colocalization between SBP-EGFP-ShhN and GM130 in the juxtanuclear area labelled by SBP-EGFP-ShhN. ****, p<0.0001.

FIGS. 6A-6D. Synthesis of proteoglycans regulates TGN-to-cell surface delivery of SBP-EGFP-Shh^{FL}. FIG. 6A. HeLa cells were untreated (FIG. 6A Panels A-F) or treated (FIG. 6A Panels G-I) with 2.5 mM xyloside. 24 hr after xyloside treatment, cells were transfected with plasmids encoding Str-KDEL_SBP-EGFP-HA-Shh^{FL}. 48 hr after xyloside treatment, cells were treated without biotin (FIG. 6A Panels A-C) or with biotin for 1 hr (FIG. 6A Panels D-I). Then the antibody uptake assay was performed using mouse anti-HA antibodies. The surface and total SBP-EGFP-HA-Shh^{FL} were detected by immunofluorescence. Scale bar, 10 μm. Magnification, 63×. FIG. 6B. Quantification of the percentage of cells showing surface-localized Shh (mean±S.D.; n=3; >50 cells counted for each group). ***, p<0.001. FIG. 6C. HeLa cells were transfected with NC siRNA or two different siRNAs against XYLT2. 48 hr after transfection, cells were re-transfected with plasmids encoding Str-KDEL and SBP-EGFP-Shh^{FL}. On day 3 after knockdown, cells were treated with biotin and incubated at 20° C. for 2 hr. Cells were then incubated at 32° C. for 0 or 45 min and the localizations of Shh constructs were analyzed. Scale bar, 10 μm. Magnification, 63×. The magnified views of the indicated areas in panels N-P are shown in panels N′-P′. FIG. 6D. Quantifications of the number of punctate structures containing SBP-EGFP-Shh^{FL} per cell at different time points after biotin treatment (n=3, mean±S.D., over 20 cells were quantified in each experimental group). **, p<0.01; ***, p<0.001.

FIGS. 7A-7F. Residues 33-49 in ShhN are important for ER export of ShhN. FIG. 7A. Diagram demonstrating the RUSH assay. FIG. 7B. HeLa cells were transfected with plasmids encoding SBP-EGFP-ShhN²⁵⁻¹⁹⁸ and Streptavidin-KDEL. Day 1 after transfection, cells were left untreated or treated with biotin for 15 min. The localizations of the indicated proteins were then analyzed by immunofluorescence. Scale bar, 10 [symbol] m. Magnification, 63×. FIG. 7C. Quantification showing that ShhN colocalized with TGN46 among all of the cells showing juxta-nuclear located pattern of SBP-EGFP-ShhN 15 min after biotin treatment (n=3). FIG. 7D. HeLa cells were transfected with plasmids encoding Str-KDEL and SBP-EGFP-ShhN²⁵⁻¹⁹⁸ (FIG. 7D Panels K-M) or SBP-EGFP (FIG. 7D Panels N-P) or SBP-EGFP tagged different truncated versions of ShhN (FIG. 7D Panels Q-AE). Day 1 after transfection, the localization of the different versions of RUSH constructs containing ShhN was analyzed after incubation with biotin for the indicated time. Scale bar, 10 μm. Magnification, 63×. FIG. 7E. Quantification of the percentage of cells showing juxta-nuclear-accumulated EGFP signal 30 min after biotin treatment (mean±S.D.; n=3; >100 cells counted for each time point). FIG. 7F. Diagram depicting the fragment of ShhN that can be transported to the Golgi.

FIGS. 8A-8D. SURF4 was identified to be a binding partner of ShhN. FIGS. 8A-8B. HEK293T cells were left un-transfected or transfected with plasmids encoding ShhN¹⁻¹⁹⁸-HA (ShhN-HA) or IGF2-HA. Day 1 after transfection, cells were lysed and ShhN-HA or IGF2-HA were immunoprecipitated and analyzed by SDS-PAGE and Coomassie blue staining (FIG. 8A) or immunoblotting with anti-HA antibodies (FIG. 8B). Asterisks indicate the position of IGF2-HA or ShhN-HA. FIG. 8C. A label-free quantification was performed to quantify the intensity of unique peptides to measure the abundance of proteins identified in the mass spectrometry. A table showing the list of proteins in the eluted fraction whose abundance in the ShhN-HA group was calculated to be more than 3-fold that detected in the IGF2-HA group. FIG. 8D. HEK293T cells were transfected with plasmids encoding the constructs indicated. Day 1 after transfection, cells were treated with 2 mM DSP, and cell lysates were incubated with beads conjugated with anti-HA antibodies. The bound proteins indicated were analyzed by immunoblotting with anti-HA or anti-Myc antibodies.

FIGS. 9A-9B. Expressing the siRNA resistant SURF4 rescues the defects of ER-to-Golgi transport of ShhN in SURF4 knockdown cells. FIG. 9A Panels A-R. HeLa cells were transfected with negative control (NC) siRNA (FIG. 9A Panels A-B, G-H and M-N) or siRNAs against SURF4 (FIG. 9A Panels C-F, I-L and O-R). 48 hr after transfection, cells were re-transfected with plasmids encoding Str-KDEL_SBP-EGFP-ShhN²⁵⁻¹⁹⁸ (FIG. 9A Panels A-D, G-J and M-P), or siRNA-resistant SURF4^{RS}-HA and Str-KDEL_SBP-EGFP-ShhN²⁵⁻¹⁹⁸ (FIG. 9A Panels E-F, K-L and Q-R). On day 3 after knockdown, cells were incubated with biotin for the indicated time and the localization of ShhN was analyzed. Scale bar, 10 μm. Magnification, 63×. FIG. 9B. Quantification of the percentage of cells showing juxta-nuclear-localized SBP-EGFP-ShhN²⁵⁻¹⁹⁸ (n=4, mean±S.D., over 100 cells were quantified in each experimental group). **, p<0.01; n.s., not significant.

FIGS. 10A-10B. SURF4 regulates ER-to-Golgi trafficking of SBP-EGFP-ShhN³³⁻³⁹ FIG. 10A. HeLa cells were transfected with negative control (NC) siRNA (FIG. 10A Panels A, D) or two different siRNAs against SURF4 (FIG. 10A Panels B, E, C, F). 24 hr after transfection, cells were re-transfected with plasmids encoding SBP-EGFP-ShhN³³⁻³⁹ and Str-KDEL. On day 3 after knockdown, cells were incubated with biotin for the indicated time and the localization of the indicated proteins was analyzed. Scale bar, 10 μm. Magnification, 63×. FIG. 10B. Quantifications of the percentage of cells showing juxta-nuclear-localized SBP-EGFP-ShhN³³⁻³⁹ (mean±S.D.; n=3; >100 cells counted for each experiment). **, p<0.01. Note: Shh used in this figure is from mouse.

FIGS. 11A-11C. Knockdown of SURF4 did not cause defects in ER-to-Golgi transport and the secretion of IGF2. FIG. 11A Panels A-F. HeLa cells were transfected with negative control (NC) siRNA (FIG. 11A Panels A-C) or siRNAs against SURF4 (FIG. 11A Panels D-F). 48 hr after transfection, cells were re-transfected with plasmids encoding Str-KDEL_SBP-EGFP-IGF2. On day 3 after knockdown, cells were incubated with biotin for the indicated time and the localization of IGF2 was analyzed. Scale bar, 10 μm. Magnification, 63×. FIG. 11B. Quantification of the percentage of cells showing juxta-nuclear-localized SBP-EGFP-IGF2 (n=3, mean±S.D., over 100 cells were quantified in each experimental group). FIG. 11C. HEK293T cells were transfected with control siRNA or siRNA against SURF4. 24 hr after transfection, cells were re-transfected with plasmids encoding Str-KDEL and SBP-EGFP-IGF2-HA. On day 3 after knockdown, cells were incubated with biotin for 2 hr. After biotin incubation, the level of SBP-EGFP-IGF2-HA in the medium and in cell lysates was analyzed by immunoblotting with anti-HA or anti-SEC22B antibodies.

FIG. 12. Analysis of the colocalization between SBP-EGFP-ShhN and SURF4 or SEC31A using a permeabilized cell assay. Panels A-O. HeLa cells were transfected with SBP-EGFP-ShhN²⁵⁻¹⁹⁸ (referred to as SBP-EGFP-ShhN). Day 1 after transfection, cells were untreated (Panels A-C) or treated with biotin for 4 min (Panels D-O). Subsequently, cells were permeabilized by digitonin and incubated with the indicated reagents. After incubation, the localization of the indicated proteins was analyzed by immunofluorescence. Size Bar, 10 [symbol] m. Magnified views of the indicated areas in panels F and I are shown in panels F′-F′″ and I′-I′″. Magnified views of the indicated areas in panel O are shown in panels O′-P′″.

FIGS. 13A-13H. SURF4 traffics together with ShhN from the ER to the Golgi. FIG. 13A Panels A-I. HeLa cells were co-transfected with SBP-EGFP-ShhN²⁵⁻¹⁹⁸ (SBP-EGFP-ShhN) and SURF4-HA. Day 1 after transfection, the localizations of the indicated proteins were analyzed 0 min (FIG. 13A Panels A-C), 20 min (FIG. 13A Panels D-F) or 60 min (FIG. 13A Panels G-I) after biotin treatment. FIG. 13B. Quantification of the percentage of cells showing Golgi-localized SURF4-HA in cells co-expressing SURF4-HA and SBP-EGFP-ShhN 0 min or 20 min after biotin treatment (n=3, mean±S.D., over 100 cells were quantified in each experimental group). **, p<0.01. FIG. 13C Panels K-M. The localizations of ERGIC53 and GM130 were analyzed in Hela cells. FIG. 13D. Quantification of the colocalization between GM130 and ERGIC53 (n=3, mean±S.D., over 20 cells were quantified in each experimental group). FIG. 13E Panels O-V. HeLa cells co-transfected with SURF4-HA and SBP-EGFP-ShhN were treated with biotin for 20 min, and the localizations of the indicated proteins were analyzed. Scale bar, 10 μm. Magnification, 63×. FIG. 13F. Quantification of the colocalization between SURF4-HA and ERGIC53 or GM130 (n=3, mean±S.D., over 20 cells were quantified in each experimental group). ****, p<0.0001. FIG. 13G. HEK293T cells were co-transfected with plasmids encoding SURF4-Myc and SBP-EGFP-ShhN-HA. Day 1 after transfection, cells were incubated at 20° C. for 2 hr in the absence or presence of biotin. Then the cells were treated in 2 mM DSP, and the cell lysates were incubated with beads conjugated with HA antibodies. The bound proteins were analyzed by immunoblotting with anti-HA or anti-Myc antibodies. FIG. 13H. Relative levels of SURF4-Myc that bound to SBP-EGFP-ShhN-HA were quantified (n=3, mean±S.D.). *, p<0.05.

FIGS. 14A-14B. Xyloside treatment did not cause defects in ER-to-Golgi transport of ShhN. FIG. 14A Panels A-D. HeLa cells were untreated or treated with 2.5 mM xyloside. 24 hr after xyloside treatment, cells were transfected with plasmids encoding Str-KDEL and SPB-EGFP-ShhN²⁵⁻¹⁹⁸ (SBP-EGFP-ShhN). 48 hr after xyloside treatment, cells were incubated with biotin for the indicated time. After biotin incubation, the localization of SBP-EGFP-ShhN was analyzed. Scale bar, 10 μm. Magnification, 63×. FIG. 14B. Quantification of the percentage of cells showing juxta-nuclear-localized SBP-EGFP-ShhN (n=3, mean±S.D., over 100 cells were quantified in each experimental group). n.s., non-significant.

FIGS. 15A-15C. Live imaging analysis of the surface delivery of SBP-EGFP-ShhN. FIG. 15A Panels A-P″. HeLa cells were untreated or treated with 2.5 mM xyloside. 24 hr after xyloside treatment, cells were transfected with plasmids encoding Str-KDEL and SBP-EGFP-ShhN²⁵⁻¹⁹⁸ (SBP-EGFP-ShhN). 48 hr after xyloside treatment, a time-lapse series of confocal images of SBP-EGFP-ShhN following biotin addition were acquired at an interval of 30 seconds. Representative images at selected time points are shown. The magnified views of the indicated areas in panels C-D, G-H, K-L, and O-P are shown in panels C′-D″, G′-H″, K′-L″, O′-P″. Scale bar, 10 μm. Magnification, 63×. FIG. 15B. Quantifications of number of punctate structures of SBP-EGFP-ShhN per cell at different time points after biotin treatment. FIG. 15C. Quantifications of number of punctate structures containing SBP-EGFP-ShhN per cell at 50 min after biotin treatment (mean±S.D., each dot represents one cell). **, p<0.01.

FIGS. 16A-16D. Lowing the pH to 6.0 did not promote the release of SURF4 from ShhN. FIG. 16A. Purified ShhN²⁵⁻⁴⁹-GST was incubated with lysates from HEK293T cells transfected with SURF4-HA at pH 6.0 or pH 7.2. After incubation, the bound proteins were analyzed by immunoblotting with anti-HA antibodies. FIG. 16B. Relative levels of SURF4-HA that bound to ShhN²⁵⁻⁴⁹-GST were quantified (n=3, mean±S.D.). The level of SURF4-HA that bound to ShhN²⁵⁻⁴⁹-GST at pH7.2 was normalized to the corresponding bait protein, and this value was then normalized to the level of SURF4-HA that bound to ShhN²⁵⁻⁴⁹-GST at pH6.0 in each experimental group. n.s., not significant. FIG. 16C. Purified ShhN²⁵⁻⁴⁹-GST was incubated with lysates from HEK293T cells transfected with SURF4-HA in the presence of the indicated concentrations of heparin at pH6.0. After incubation, the bound proteins were analyzed by immunoblotting with anti-HA antibodies. FIG. 16D. Relative levels of SURF4-HA that bound to ShhN²⁵⁻⁴⁹-GST were quantified (n=3, mean±S.D.). The quantification is normalized to the level of SURF4-HA that bound to ShhN²⁵⁻⁴⁹-GST in the absence of Heparin in each experimental group. *, p<0.05.

FIGS. 17A-17D. SBP-EGFP-Shh^{FL} is processed into N-terminal and C-terminal fragments. FIG. 17A. HA-tagged Shh^{FL} in cell lysates from HEK293T cells expressing SBP-EGFP-HA-Shh^{FL} (HA-Shh^{FL}) or SBP-EGFP-Shh^{FL}-HA (Shh^{FL}-HA) was analyzed by immunoblotting using anti-HA antibodies. FIG. 17B. HEK293T cells transfected with plasmids encoding Str-KDEL and SBP-EGFP-HA-Shh^{FL} were incubated in the presence or absence of biotin for 2 hr. After incubation, the level of Shh in the medium and in cell lysates was then analyzed by immunoblotting using anti-HA antibodies. FIG. 17C. HeLa cells were transfected with plasmids encoding Str-KDEL and SBP-EGFP-Shh^{FL}-HA. Day 1 after transfection, the localizations of the indicated proteins were analyzed 0 min (FIG. 17C panels C-E) or 20 min (FIG. 17C panels F-H) after biotin treatment. Scale bar, 10 μm. Magnification, 63×. FIG. 17D. Quantification of the percentage of cells showing juxta-nuclear-localized SBP-EGFP-ShhN and ShhC-HA 0 min or 20 min after biotin treatment (n=3, mean±S.D., over 100 cells were quantified in each experimental group). ****, p<0.0001.

FIGS. 18A-18D. SURF4 regulates ER-to-Golgi transport of SBP-EGFP-Shh^{FL}. FIG. 18A Panels A-R. HeLa cells were transfected with negative control (NC) siRNA (FIG. 18A panels A-F) or siRNAs against SURF4 (FIG. 18A panels G-R). 48 hr after transfection, cells were re-transfected with plasmids encoding Str-KDEL_ SBP-EGFP-Shh^{FL} (FIG. 18A panels A-L), or siRNA-resistant SURF4^{RS}-HA and Str-KDEL SBP-EGFP-Shh^{FL} (FIG. 18A panels M-R). On day 3 after knockdown, cells were incubated with biotin for the indicated time and the localization of Shh was analyzed. FIG. 18B. Wild type (wt) or SURF4 KO HEKTrex cells were transfected with plasmids encoding Str-KDEL_SBP-EGFP-Shh^{FL} (FIG. 18B panels S-X), or re-transfected with plasmids encoding the siRNA-resistant SURF4^{RS}-HA and Str-KDEL SBP-EGFP-Shh^{FL} (FIG. 18B panels Y-AA). On day 3 after knockdown, cells were incubated with biotin for 15 min and the localizations of the indicated proteins were analyzed. Scale bar, 10 μm. Magnification, 63×. FIGS. 18C-18D. Quantification of the percentage of cells showing juxta-nuclear-localized SBP-EGFP-ShhFL (n=3, mean±S.D., over 100 cells were quantified in each experimental group). **, p<0.01; ***, p<0.001.

FIG. 19A-19B. Blocking synthesis of proteoglycan does not inhibit the surface delivery of SBP-EGFP-E-cadherin. FIG. 19A panels A-L. HeLa cells were untreated (FIG. 19A panels A-F) or treated with 2.5 mM xyloside (FIG. 19A panels G-L). 24 hr after xyloside treatment, cells were transfected with plasmids encoding SBP-EGFP-E-cadherin. On day 3 after xyloside treatment, cells were treated with biotin and incubated in the 20° C. for 2 hr. Then the cells were incubated in 32° C. for 0 min (FIG. 19A panels A-C, G-I) or 45 min (FIG. 19A panels D-F, J-L). The localizations of the indicated constructs were analyzed. Size bar, 10 μm. FIG. 19B. Quantifications of the percentage of cells showing surface-localized SBP-EGFP-E-cadherin (n=3, mean±S.D., over 100 cells were quantified in each experimental group). n.s. not significant.

FIG. 20. A diagram demonstrating the invention.

### BRIEF DESCRIPTION OF THE SEQUENCES

SEQ ID NO: 1: SURF4 sgRNA

SEQ ID NO: 2: siRNA against SURF4

SEQ ID NO: 3: siRNA against SURF4

SEQ ID NO: 4: siRNA against XYLT2

SEQ ID NO: 5: siRNA against XYLT2

SEQ ID NO: 6: Cardin-Weintraub peptide

SEQ ID NO: 7: Cardin-Weintraub peptide

SEQ ID NO: 8: Cardin-Weintraub peptide

SEQ ID NO: 9: Cardin-Weintraub peptide^{MT} peptide

SEQ ID NO: 10: SURF4 peptide

SEQ ID NO: 11: RRFR peptide

SEQ ID NO: 12: SURF4 peptide

SEQ ID NO: 13: Nucleotide sequence encoding Cardin-Weintraub peptide of SEQ ID NO: 8

SEQ ID NO: 14: Nucleotide sequence encoding SURF4 peptide of SEQ ID NO: 10

SEQ ID NO: 15: Human SURF4 amino acid sequence

SEQ ID NO: 16: Human SURF4 cDNA nucleotide sequence

SEQ ID NO: 17: Human Sonic Hedgehog amino acid sequence

SEQ ID NO: 18: Human Sonic Hedgehog cDNA nucleotide sequence

### DETAILED DISCLOSURE OF THE INVENTION

### Selected Definitions

As used herein, the singular forms “a,” “an,” and “the” are intended to include the plural forms as well, unless the context clearly indicates otherwise. Further, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” “comprise,” include the phrases “consisting essentially of,” “consists essentially of,” “consisting,” and “consists.”

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which will depend in part on how the value is measured or determined, i.e., the limitations of the measurement system. Where particular values are described in the application and claims, unless otherwise stated the term “about” meaning within an acceptable error range for the particular value should be assumed.

In the present disclosure, ranges are stated in shorthand, to avoid having to set out at length and describe each and every value within the range. Any appropriate value within the range can be selected, where appropriate, as the upper value, lower value, or the terminus of the range. For example, a range of 1-10 represents the terminal values of 1 and 10, as well as the intermediate values of 2, 3, 4, 5, 6, 7, 8, 9, and all intermediate ranges encompassed within 1-10, such as 2-5, 2-8, and 7-10. Also, when ranges are used herein, combinations and sub-combinations of ranges (e.g., subranges within the disclosed range) and specific embodiments therein are intended to be explicitly included.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

As used herein, an “isolated” or “purified” nucleic acid molecule, polynucleotide, polypeptide, protein or organic compound such as a large molecule (e.g., those described below), is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. An isolated microbial strain means that the strain is removed from the environment in which it exists in nature. Thus, the isolated strain may exist as, for example, a biologically pure culture, or as spores (or other forms of the strain) in association with a carrier.

As used herein, the term “large molecule” refers to a biologic, including, for example, a protein, peptide, antibody, or blood component.

As used herein, the term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

In this application, the terms “polypeptide”, “peptide”, and “protein” are used interchangeably herein to refer to a polymer of amino acids. The terms apply to amino acid polymers in which one or more amino acid residues are artificial chemical mimetic of a corresponding naturally occurring amino acids, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used herein, “vector” refers to a DNA molecule such as a plasmid for introducing a nucleotide construct, for example, a DNA construct, into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide a selectable characteristic, such as tetracycline resistance, hygromycin resistance or ampicillin resistance.

As used in herein, the terms “identical” or “percent identity”, in the context of describing two or more polynucleotide sequences, refer to two or more sequences or subsequences that are the same or have a specified percentage of nucleotides that are the same over the compared region. For example, a homologous nucleotide sequence used in the method of this invention has at least 80% sequence identity, preferably 85%, 90%, 91%, 92%, 93, 94%, 95%, 96%, 97%, 98%, 99%, or 100% identity, to a reference sequence, when compared and aligned for maximum correspondence over a comparison window, or over a designated region as measured using a comparison algorithms or by manual alignment and visual inspection. With regard to polynucleotide sequences, this definition also refers to the complement of a test sequence.

An endogenous nucleic acid is a nucleic acid that is naturally present in a cell. For example, a nucleic acid present in the genomic DNA of a cell is an endogenous nucleic acid.

An exogenous nucleic acid is any nucleic acid that is not naturally present in a cell. For example, a nucleic acid vector introduced into a cell constitutes an exogenous nucleic acid. Other examples of an exogenous nucleic acid include the vectors comprising a heterologous promoter linked to an endogenous nucleic acid, e.g., a nucleic acid encoding a kinase.

The subject invention provides for the use of “homologous amino acid sequences” or “homologs of amino acid sequences”. Homologs of amino acid sequences will be understood to mean any amino sequence obtained by mutagenesis, particularly mutagenesis of the encoding nucleotide sequence, according to techniques well known to persons skilled in the art, and exhibiting modifications in relation to the parent sequences. For example, mutations in the regulatory and/or promoter sequences for the expression of a polypeptide that result in a modification of the level of expression of a polypeptide according to the invention provide for a “homolog of an amino acid sequence”. Likewise, substitutions, deletions, or additions of nucleic acid to the polynucleotides of the invention provide for “homologs” of encoded amino acid sequences. In various embodiments, “homologs” of amino acid sequences have substantially the same biological activity as the corresponding reference amino acid sequence, i.e., a protein homologous to a native protein+ [symbol] having the same biological activity as the naturally occurring protein. Typically, a homolog of an amino acid sequence shares a sequence identity with the gene of at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. These percentages are purely statistical and differences between two amino acid sequences can be distributed randomly and over the entire sequence length.

The subject invention provides for the use of “homologous nucleic acid sequences” or “homologs of nucleic acid sequences”. Homologs of nucleic acid sequences will be understood to mean any nucleotide sequence obtained by mutagenesis according to techniques well known to persons skilled in the art, and exhibiting modifications in relation to the parent sequences. For example, mutations in the regulatory and/or promoter sequences for the expression of a polypeptide that result in a modification of the level of expression of a polypeptide according to the invention provide for a “homolog of a nucleotide sequence”. Likewise, substitutions, deletions, or additions of nucleic acid to the polynucleotides of the invention provide for “homologs” of nucleotide sequences. In various embodiments, “homologs” of nucleic acid sequences have substantially the same biological activity as the corresponding reference gene, i.e., a gene homologous to a native gene would encode for a protein having the same biological activity as the corresponding protein encoded by the naturally occurring gene. Typically, a homolog of a gene shares a sequence identity with the gene of at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. These percentages are purely statistical and differences between two nucleic acid sequences can be distributed randomly and over the entire sequence length.

The phrase “a transformed cell” as used herein refers to a cell in which the cells are transformed with a DNA vector or plasmid disclosed herein.

In certain embodiments, purified compounds are at least 60% by weight the compound of interest. Preferably, the preparation is at least 75%, more preferably at least 90%, and most preferably at least 98%, by weight the compound of interest. For example, a purified compound is one that is at least 80%, 85%, 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

As used herein a “reduction” means a negative alteration, and an “increase” means a positive alteration, wherein the negative or positive alteration is at least 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

As used herein, the term “effective amount” is used to refer to an amount of a compound or composition that, when applied or contacted to an organism, is capable of inhibiting, preventing, or improving a condition in a subject. In other words, when applied or contacted to an organism, the amount is “effective.” The actual amount will vary depending on a number of factors including, but not limited to, the severity of the condition and the route of application.

“Treating” or “treatment” of any cancer refers, in one embodiment, to ameliorating the cancer (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the cancer, either physically, (e.g., stabilization of a discernible symptom), physiologically, (e.g., stabilization of a physical parameter), or both. In yet another embodiment, “treating” or “treatment” refers to delaying the onset of the cancer.

As used herein, the terms “reducing”, “inhibiting”, “blocking”, “preventing”, “alleviating”, or “relieving” when referring to a polypeptide or other compound, mean that the polypeptide or other compound brings down the occurrence, severity, size, volume or associated symptoms of cancer by at least about 7.5%, 10%, 12.5%, 15%, 17.5%, 20%, 22.5%, 25%, 27.5%, 30%, 35%, 40%, 45%, 50%, 55%, 60%, 65%, 70%, 75%, 80%, 90%, or 100% compared to how the cancer would normally exist without application of the polypeptide or other compound or a composition comprising the polypeptide or other compound.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references cited herein are hereby incorporated by reference in their entirety.

### Compositions that Reduce the Interaction of Shh and SURF4

The present disclosure is related to various compounds that inhibit the interaction between Shh and SURF4 in a subject, particularly in a subject suffering from cancer or to delay the onset of cancer in a subject. In certain embodiments, the methods according to the invention comprise the administration of a therapeutically effective amount of a novel composition comprising a polypeptide and/or a different molecule (e.g., large molecule) to a subject suffering from or diagnosed as having cancer. A “therapeutically effective amount” means an amount or dose sufficient to generally bring about the desired therapeutic or prophylactic benefit in patients in need of such treatment for the designated cancer.

In certain embodiments, a novel composition inhibits the Shh and SURF4 interaction and comprises an effective amount of a polypeptide with an amino acid comprising SEQ ID NOs: 6-8 or SEQ ID NO: 10. Furthermore, the effective amount of the composition would comprise a polypeptide with an amino acid sequence with a 90% identity or greater to SEQ ID NOs: 6-8 or SEQ ID NO: 10.

In certain embodiments, a vector can be readily prepared using methods available in the art. The transformation vector comprises one or more nucleotide sequences that is/are capable of being transcribed to an RNA molecule and that is/are substantially homologous and/or complementary to one or more nucleotide sequences encoding amino acid sequences SEQ ID NOs: 6-8 or SEQ ID NO: 10. A recombinant nucleic acid vector may, for example, be a linear or a closed circular plasmid. The vector system may be a single vector or plasmid or two or more vectors or plasmids that together contain the total nucleic acid. The vector may encode a nucleotide sequence that encodes a polypeptide that targets Shh, SURF4, and/or the interaction of Shh and SURF4.

In certain embodiments, the novel compositions can comprise molecules, such as, for example, a glycosaminoglycan (GAG). In certain embodiments, the GAG is, for example, heparin, heparin sulfate, heparin sulfate proteoglycans (HSPG), or chondroitin sulfate proteoglycans (CSPGs).

Effective amounts or doses of the polypeptides or other molecules (e.g., large molecule) of the present invention may be ascertained by routine methods such as modeling, dose escalation studies or clinical trials, and by taking into consideration routine factors, e.g., the mode or route of administration or drug delivery, the pharmacokinetics of the polypeptide or other (e.g., large molecule) molecule, the severity and course of cancer, the subject's previous or ongoing therapy, the subject's health status and response to drugs, and the judgment of the treating physician. An example of a dose is in the range of from about 0.001 to about 200 mg of a polypeptide or other molecule (e.g., large molecule) per kg of the subject's body weight per day, preferably about 0.05 to 100 mg/kg/day, or about 1 to 35 mg/kg/day, even more preferably, about 1, 5, 10, or 20 mg/kg/day, in single or divided dosage units (e.g., BID, TID, QID). For a 70-kg human, an illustrative range for a suitable dosage amount is from about 0.05 to about 7 g/day, preferably, about 0.07 to about 2.45 g/day, even more preferably, about 0.07, 0.35, 0.7, or 1.4 g/day.

In certain embodiments, the polypeptides or other molecules (e.g., large molecule) can be administered intramuscularly, subcutaneously, intrathecally, intravenously or intraperitoneally by infusion or injection. Solutions of the polypeptides or other molecules (e.g., large molecule) can be prepared in water, optionally mixed with a nontoxic surfactant. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the polypeptides or other molecules (e.g., large molecule) that are adapted for the extemporaneous preparation of sterile injectable or infusible solutions or dispersions, optionally encapsulated in liposomes. Preferably, the ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained by, for example, the formation of liposomes, by the maintenance of the required particle size in the case of dispersions, or by the use of surfactants. The prevention of the action of microorganisms can be brought about by various antibacterial and antifungal agents, for example, parabens, chlorobutanol, phenol, sorbic acid, thimerosal, and the like. In many cases, it is preferable to include isotonic agents, for example, sugars, buffers, or sodium chloride. Prolonged absorption of the injectable compositions can be brought about by the use in the compositions of agents delaying absorption, for example, aluminum monostearate and gelatin.

Sterile injectable solutions are prepared by incorporating the polypeptides or other molecules (e.g., large molecule) in the required amount in the appropriate solvent as described herein with various of the other ingredients enumerated herein, as required, preferably followed by filter sterilization. In the case of sterile powders for the preparation of sterile injectable solutions, the preferred methods of preparation are vacuum drying and freeze drying techniques, which yield a powder of the active ingredient plus any additional desired ingredient present in the previously sterile-filtered solutions.

The compositions of the subject invention may also be administered orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the subject's diet.

For oral therapeutic administration, the polypeptides or other molecules (e.g., large molecule) can be combined with one or more excipients and used in the form of ingestible tablets, buccal tablets, troches, capsules, elixirs, suspensions, syrups, wafers, and the like. Such compositions and preparations should contain, for example, at least 0.1% of a polypeptides or other molecules of the present invention. The percentage of the polypeptides or other molecules (e.g., large molecule) of the invention present in such compositions and preparations may, of course, be varied. The amount of the polypeptides or other molecules (e.g., large molecule) in such therapeutically useful compositions is such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid, and the like; a lubricant such as magnesium stearate; and a sweetening agent such as sucrose, fructose, lactose, or aspartame, or a flavoring agent such as peppermint, oil of wintergreen, or cherry flavoring may be added.

When the unit dosage form is a capsule, it may contain, in addition to materials of the above type, a liquid carrier, such as a vegetable oil or a polyethylene glycol.

Various other materials may be present as coatings or for otherwise modifying the physical form of the solid unit dosage form. For instance, tablets, pills, or capsules may be coated with gelatin, wax, shellac, or sugar, and the like. A syrup or elixir may contain the polypeptides or other molecules (e.g., large molecule), sucrose or fructose as a sweetening agent, methyl and propylparabens as preservatives, a dye, and flavoring such as cherry or orange flavor.

Of course, any material used in preparing any unit dosage form should be pharmaceutically acceptable and substantially non-toxic in the amounts employed.

In addition, the polypeptides or other molecules (e.g., large molecule) may be incorporated into sustained-release preparations and devices. For example, the polypeptides or other molecules (e.g., large molecule) may be incorporated into time release capsules, time release tablets, time release pills, and time release polypeptides or other molecules (e.g., large molecule) or nanoparticles.

Pharmaceutical compositions for topical administration of the polypeptides or other molecules (e.g., large molecule) to the epidermis (mucosal or cutaneous surfaces) can be formulated as ointments, creams, lotions, gels, or as a transdermal patch. Such transdermal patches can contain penetration enhancers such as linalool, carvacrol, thymol, citral, menthol, t-anethole, and the like. Ointments and creams can, for example, include an aqueous or oily base with the addition of suitable thickening agents, gelling agents, colorants, and the like. Lotions and creams can include an aqueous or oily base and typically also contain one or more emulsifying agents, stabilizing agents, dispersing agents, suspending agents, thickening agents, coloring agents, and the like. Gels preferably include an aqueous carrier base and include a gelling agent such as cross-linked polyacrylic acid polymer, a derivatized polysaccharide (e.g., carboxymethyl cellulose), and the like.

Pharmaceutical compositions suitable for topical administration in the mouth (e.g., buccal or sublingual administration) include lozenges comprising the composition in a flavored base, such as sucrose, acacia, or tragacanth; pastilles comprising the composition in an inert base such as gelatin and glycerin or sucrose and acacia; and mouthwashes comprising the active ingredient in a suitable liquid carrier. The pharmaceutical compositions for topical administration in the mouth can include penetration enhancing agents, if desired.

Useful solid carriers include finely divided solids such as talc, clay, microcrystalline cellulose, silica, alumina, and the like. Other solid carriers include nontoxic polymeric nanoparticles or microparticles. Useful liquid carriers include water, alcohols, or glycols, or water/alcohol/glycol blends, in which the polypeptides or other molecules (e.g., large molecule) can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of useful dermatological compositions which can be used to deliver the polypeptides or other molecules (e.g., large molecule) to the skin are known in the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508), all of which are hereby incorporated by reference.

The concentration of the polypeptides or other molecules (e.g., large molecule) of the invention in such formulations can vary widely depending on the nature of formulation and intended route of administration. For example, the concentration of the polypeptides or other molecules (e.g., large molecule) in a liquid composition, such as a lotion, can preferably be from about 0.1-25% by weight, or, more preferably, from about 0.5-10% by weight. The concentration in a semi-solid or solid composition such as a gel or a powder can preferably be about 0.1-5% by weight, or, more preferably, about 0.5-2.5% by weight.

Pharmaceutical compositions for spinal administration or injection into amniotic fluid can be provided in unit dose form in ampoules, pre-filled syringes, small volume infusion, or in multi-dose containers, and can include an added preservative. The compositions for parenteral administration can be suspensions, solutions, or emulsions, and can contain excipients such as suspending agents, stabilizing agents, and dispersing agents.

A pharmaceutical composition suitable for rectal administration comprises polypeptides or other molecules (e.g., large molecule) of the present invention in combination with a solid or semisolid (e.g., cream or paste) carrier or vehicle. For example, such rectal compositions can be provided as unit dose suppositories. Suitable carriers or vehicles include cocoa butter and other materials commonly used in the art.

According to one embodiment, pharmaceutical compositions of the present invention suitable for vaginal administration are provided as pessaries, tampons, creams, gels, pastes, foams, or sprays containing polypeptides or other molecules (e.g., large molecule) of the invention in combination with carriers as are known in the art. Alternatively, compositions suitable for vaginal administration can be delivered in a liquid or solid dosage form.

Pharmaceutical compositions suitable for intra-nasal administration are also encompassed by the present invention. Such intra-nasal compositions comprise polypeptides or other molecules (e.g., large molecule) of the invention in a vehicle and suitable administration device to deliver a liquid spray, dispersible powder, or drops. Drops may be formulated with an aqueous or non-aqueous base also comprising one or more dispersing agents, solubilizing agents, or suspending agents. Liquid sprays are conveniently delivered from a pressurized pack, an insufflator, a nebulizer, or other convenient means of delivering an aerosol comprising polypeptides or other molecules (e.g., large molecule). Pressurized packs comprise a suitable propellant such as dichlorodifluoromethane, trichlorofluoromethane, dichlorotetrafluoroethane, carbon dioxide, or other suitable gas as is well known in the art. Aerosol dosages can be controlled by providing a valve to deliver a metered amount of polypeptides or other molecules (e.g., large molecule).

The polypeptides or other molecules (e.g., large molecule) may be combined with an inert powdered carrier and inhaled by the subject or insufflated.

Pharmaceutical compositions for administration by inhalation or insufflation can be provided in the form of a dry powder composition, for example, a powder mix of polypeptides or other molecules (e.g., large molecule) and a suitable powder base such as lactose or starch. Such powder composition can be provided in unit dosage form, for example, in capsules, cartridges, gelatin packs, or blister packs, from which the powder can be administered with the aid of an inhalator or insufflator.

The exact amount (effective dose) of the polypeptides or other molecules (e.g., large molecule) varies from subject to subject, depending on, for example, the species, age, weight, and general or clinical condition of the subject, the severity or mechanism of any cancer being treated, the particular agent or vehicle used, the method and scheduling of administration, and the like. A therapeutically effective dose can be determined empirically, by conventional procedures known to those of skill in the art. See, e.g., The Pharmacological Basis of Therapeutics, Goodman and Gilman, eds., Macmillan Publishing Co., New York. For example, an effective dose can be estimated initially either via in vivo assays or in suitable animal models. The animal model may also be used to determine the appropriate concentration ranges and routes of administration. Such information can then be used to determine useful doses and routes for administration in humans. Methods for the extrapolation of effective dosages in mice and other animals to humans are known to the art; for example, see U.S. Pat. No. 4,938,949, which is hereby incorporated by reference. A therapeutic dose can also be selected by analogy to dosages for comparable therapeutic agents.

The particular mode of administration and the dosage regimen can be selected by the attending clinician, taking into account the particulars of the case (e.g., the subject, the disease, the disease state involved, and whether the treatment is prophylactic). Treatment may involve daily or multi-daily doses of compound(s) over a period of a few days to months, or even years.

In general, however, a suitable dose can be in the range of from about 0.001 to about 100 mg/kg of body weight per day, preferably from about 0.01 to about 100 mg/kg of body weight per day, more preferably, from about 0.1 to about 50 mg/kg of body weight per day, or even more preferred, in a range of from about 1 to about 10 mg/kg of body weight per day. For example, a suitable dose may be about 1 mg/kg, 10 mg/kg, or 50 mg/kg of body weight per day.

The polypeptides or other molecules (e.g., large molecule) can be conveniently administered in unit dosage form, containing for example, about 0.05 to about 10000 mg, about 0.5 to about 10000 mg, about 5 to about 1000 mg, or about 50 to about 500 mg of active ingredient per unit dosage form.

The polypeptides or other molecules (e.g., large molecule) can be included in the compositions within a therapeutically useful and effective concentration range, as determined by routine methods that are well known in the medical and pharmaceutical arts.

The polypeptides or other molecules (e.g., large molecule) may conveniently be presented in a single dose or as divided doses administered at appropriate intervals, for example, as one dose per day or as two, three, four or more sub-doses per day. The sub-dose itself may be further divided, e.g., into a number of discrete loosely spaced administrations; such as multiple inhalations from an insufflator.

Optionally, the pharmaceutical compositions of the present invention can include one or more other therapeutic agents, e.g., as a combination therapy. The additional therapeutic agent(s) will be included in the compositions within a therapeutically useful and effective concentration range, as determined by routine methods that are well known in the medical and pharmaceutical arts. The concentration of any particular additional therapeutic agent may be in the same range as is typical for use of that agent as a monotherapy, or the concentration may be lower than a typical monotherapy concentration if there is a synergy when combined with polypeptides or other molecules (e.g., large molecule) of the present invention.

### Methods of Inhibiting Shh and SURF4 Interaction

The present disclosure relates to treating various types of cancer in a subject, particularly ligand-dependent cancer types including, for example, stomach, esophageal, pancreatic, colorectal, ovarian and endometrial, breast, prostate, lung, melanomas, gliomas, other extracutaneous tumors, or any combination thereof, by inhibiting the Shh and SURF4 interaction.

In certain embodiments, the polypeptides and/or other molecules (e.g., large molecule) can be used for cancer treatment.

Methods for treating or preventing cancer can be performed in any subject, such as a mammal, including humans. Such methods comprise administering to a subject in need of such prevention or treatment of cancer an effective amount of a polypeptide or other molecule (e.g., large molecule) of the subject invention. A polypeptide or other molecule (e.g., large molecule) can be administered in the form of a pharmaceutical composition of a polypeptide, a molecule (e.g., large molecule), or a combination thereof.

In certain embodiments, the present invention provides methods for inhibiting the interaction between Shh and SURF4 by mutating the nucleotide sequence encoding Shh and/or SURF4 through CRISPR/Cas9 technology. In preferred embodiments, amino acid residues E50, D53, D56, or any combination thereof of human SURF4 are mutated or the nucleotide sequences encoding amino acid residues E50, D53, D56, or any combination thereof of human SURF4 are mutated. In preferred embodiments, 1, 2, 3, 4, 5, 6, or 7 residues at the position of 32-38 on human Shh are mutated or the nucleotide sequence encoding the 1, 2, 3, 4, 5, 6, or 7 residues at the position of 32-38 on human Shh are mutated.

The methods can further encompass the altering the expression of SURF4 or Shh, so as to achieve down-regulation or inhibition of one or multiple target amino acids or to achieve inhibition of the Shh and SURF4 interaction.

In certain embodiments, transcriptional suppression of a target gene is mediated by a gene suppression agent exhibiting substantial sequence identity to a DNA sequence of a target nucleotide sequence or the complement thereof, including promoter sequences or SURF4 and/or Shh. Inhibition of a target gene or amino acid of the present invention is sequence-specific and can comprise insertions, deletions, and single point mutations relative to the target sequence.

### MATERIALS AND METHODS

### Constructs, Reagents, Cell Culture, Transfection and Immunofluorescence

HeLa cells and HEK293T cell lines were kindly provided by the University of California-Berkeley Cell Culture Facility and were confirmed by short tandem repeat profiling. All cell lines were tested negative for Mycoplasma contamination. HeLa and HEK293T cells were cultured in Dulbecco's Modified Eagle Medium (DMEM) containing 10% fetal bovine serum and 1% penicillin streptomycin mix (Invitrogen). HEK 293Trex and HEK 293Trex SURF4 KO cell lines were maintained in Gibco DMEM containing 5 μg/ml blasticidin and 10% FBS.

Plasmids, siRNAs and antibodies are described herein. Transfection of siRNA or DNA constructs into HeLa cells, or HEK293T cells and immunofluorescence were performed as described previously (27). Images were acquired with a Zeiss Axio Observer Z1 microscope system (Carl Zeiss, Germany) equipped with an ORCA Flash 4.0 camera (Hamamatsu, Japan) or Leica STED TCS SP5 II Confocal Laser Scanning Microscope (Leica, Germany). To quantify the colocalization between ERGIC53 and GM130, the juxtanuclear area labelled by GM130 was outlined manually using the freehand selections function in Fiji as the region of interest (ROI). To quantify the colocalization between SURF4-HA and GM130 or ERGIC53, the juxtanuclear area labelled by SBP-EGFP-ShhN was outlined manually in Fiji as the ROI. To quantify the colocalization between ShhN and GM130, the juxtanuclear area labelled by ShhN was outlined manually in Fiji as the ROI. All of the pixels in the ROI were not saturated. The Pearson's R value (no threshold) in the ROI between two channels was then calculated using Coloc 2 in Fiji.

For CRISPR experiments, sgRNA sequences ligated into pX458 (pSpCas9 BB-2A-GFP) plasmids were purchased from GenScript. Transfections were performed with TransitIT-293 (Mirus Bio) per manufacturer's instructions. Clonal cell lines were derived by diluting cell suspensions to a single cell per well and expanding individual wells. Genotyping of clonal cell lines was performed by Sanger sequencing of target site PCR amplicons of genomic DNA isolated by Puregene kit (Quiagene). sgRNAs were as follows: SURF4, 5′-AGTCGCGCTGCTCGCTCCAC-3′ (SEQ ID NO: 1) targeting exon 1.

### Retention Using Selective Hook (RUSH) Assay and Antibody Uptake Assay

RUSH assays were performed as described previously (26). The antibody uptake assay was performed as described previously (53).

### Immunoprecipitation, Protein Purification, and Binding Assay

Immunoprecipitation was performed as described (27). Purification of GST-tagged ShhN²⁵⁻⁴⁹ and GST-tagged SURF⁴¹⁻⁶² was performed as described previously (54). GST pull down assays were performed as described previously (54). Peptide binding assay was performed as described previously (27).

### Sample Preparation for Label-Free Quantitative MS Analysis

This procedure was performed as described previously (26).

### In Vitro Vesicle Formation Assay

In vitro vesicular release assays were performed as described previously (27, 55).

### Plasmids, siRNAs and Antibodies

The cDNA encoding mouse ShhN, human SURF4, human IGF2 and human XYLT2 were ordered from BGI (Beijing, China). The plasmids encoding C-terminal 3xHA-tagged ShhN¹⁻¹⁹⁸, C-terminal GST-tagged Shh²⁵⁻⁴⁹, C-terminal 3xHA-tagged IGF2, C-terminal 3xHA-tagged SURF4, C-terminal 3xMyc-tagged SURF4, N-terminal GST-tagged SURF4⁴⁹⁻⁶⁰, N-terminal 3xMyc-tagged XYLT2, Str-KDEL_SBP-EGFP-ShhN²⁵⁻¹⁹⁸, Str-KDEL_SBP-EGFP-Shh^{FL}, Str-KDEL_SBP-EGFP-E-cadherin and truncated versions of ShhN were generated by standard molecular cloning procedures. The N-terminus of SBP-EGFP tag is followed by a signal sequence derived from IL-2 (56). The plasmids encoding mutated version of ShhN and SURF4 were generated by QuickChange II site-directed mutagenesis using plasmids encoding ShhN-HA, Str-KDEL_SBP-EGFP-ShhN and SURF4-3xMyc as templates. The plasmids encoding siRNA-resistant SURF4-HA were generated by QuickChange II site-directed mutagenesis using plasmid encoding SURF4-HA as template.

siRNAs against SURF4 and XYLT2 were purchased from Ribo-bio (Guangzhou, China). The target sequence of the two siRNAs against SURF4 is GCAGGAACTTCGTGCAGTA (SEQ ID NO: 2) and GCATCCGTATGTGGTTCCA (SEQ ID NO: 3), respectively. The target sequence of the two siRNAs against XYLT2 is CTGGTAGTGTGGAGCTTCA (SEQ ID NO: 4) and GCGTGCACCTGTATTTCTA (SEQ ID NO: 5) respectively. The commercial antibodies were rabbit anti-HA (Cell Signaling, catalogue number 3724), mouse anti-HA (Biolegend, catalogue number 901501), mouse anti-Myc (Cell Signaling, catalogue number 2276), mouse anti-PDI (Enzo, catalogue number ADI-SPA-891-F), sheep anti-TGN46 (BIO-RAD, catalogue number AHP500G), rabbit anti-ERGIC53 for the immunofluorescence analysis (Sigma-Aldrich, catalogue number E1031) and mouse anti-GM130 (BD Bioscience, catalogue number 610823). Rabbit anti-SEC22B antibodies and rabbit anti-ERGIC53 antibodies for the immunoblot analyses were kindly provided by Prof. Randy Schekman (University of California, Berkeley, CA, USA). Rabbit anti-SURF4 antibodies were kindly provided by Prof. Xiaowei Chen (Peking University, China). Rabbit anti-GFP antibodies were kindly provided by Prof. Robert Qi (Hong Kong University of Science and Technology, Hong Kong, SAR).

### Retention Using Selective Hook (RUSH) Assay, Antibody Uptake Assay and Permeabilized Cell Assay

RUSH assays were performed by treating Hela cells transfected with plasmids encoding Str-KDEL and different version of SBP-EGFP-ShhN in complete medium containing 40 μM biotin (Sigma-Aldrich) and 100 ng/μl cycloheximide (Sigma-Aldrich) for the indicated time. Cells were then fixed by 4% PFA mounted on glass slides by ProLong™ Gold Antifade Mountant with DAPI (Invitrogen) for microscope analysis.

To analyze the secretion of ShhN, HEK293T cells transfected with plasmids encoding Str-KDEL and different version of SBP-EGFP-ShhN were treated by 100 ng/μl cycloheximide and 40 μM biotin in medium without FBS addition for the indicated time. Then the secreted proteins were collected by TCA precipitation. The cells were collected and lysed by HKT buffer (100 mM KCl, 20 mM Hepes, pH 7.2, 0.5% Triton X-100). The bound proteins and cell lysates were analyzed by immunoblotting.

For antibody uptake assays, HeLa cells were treated (or not) with 2.5 mM xyloside in complete medium. 24 hr after xyloside treatment, cells were transfected with plasmids encoding Str-KDEL_SBP-EGFP-HA-Shh^{FL}. On day 2 after xyloside treatment, cells were incubated without biotin or with 40 μM biotin and 100 ng/μl cycloheximide in complete medium for 1 hr. After incubation, mouse anti-HA antibodies were added to the incubation medium at a 1:200 dilution to label the ShhN fusion construct that had been delivered to the cell surface. After an additional incubation for 40 min at 37° C., cells were fixed for 15 min with 4% paraformaldehyde in PBS and then a standard immunofluorescence procedure was performed using rabbit anti-GFP antibodies as the primary antibodies.

Permeabilized cell assay was performed as described previously (57). Briefly, HeLa cells transfected with Str-KDEL_SBP-EGFP-ShhN were treated with 0.04 mM biotin at 37° C. for 4 min, followed by three washes in cold KOAc buffer (110 mM KOAc, 2 mM Mg(OAc)₂, 20 mM Hepes, pH 7.2). Then cells were permeabilized by 0.03 mg/mL digitonin in KOAc buffer for 6 min at room temperature. The permeabilized cells were washed with cold KOAc buffer. After 5 min of incubation on ice with cold 0.5 M KOAc buffer followed by three washes in cold KOAc buffer to remove cytosolic proteins, the permeabilized cells were then incubated at 37° C. for 15 min in KOAc buffer containing 2 mg/ml rat liver cytosol, 0.04 mM biotin, 500 μM GDP/GTPγS, and an ATP regeneration system (40 mM creatine phosphate, 0.2 mg/ml of creatine phosphokinase, and 1 mM ATP). The cells were then washed with cold KOAc buffer, fixed, and stained with specific antibodies.

### Immunoprecipitation, Protein Purification, and Binding Assay

Immunoprecipitation of HA-tagged ShhN was performed by incubating 200 ml of 0.5 mg/ml cell lysates from HEK293T cells transfected with HA-ShhN in HKT buffer with 10 μl of compact anti-HA agarose affinity beads with mixing at 4° C. overnight. After incubation, the beads were washed 4 times with 1 ml of HK buffer (100 mM KCl, 20 mM Hepes, pH 7.2), and the bound material was analyzed by Coomassie blue staining and immunoblotting.

Binding assays between Myc-tagged SURF4 or Myc-tagged SURF4^{ED-AA} and HA-tagged mouse ShhN or mouse ShhN^{Δ33-39} were performed by treating HEK293T cells co-transfected with plasmids encoding the indicated proteins in 1×PBS containing 2 mM dithiobis[succinimidylpropionate] (DSP) and 2 mM CaCl₂ at room temperature for 30 min, and then quenched with 25 mM Tris-HCl, pH 7.5. 200 ml of 0.5 mg/ml cell lysates were incubated with 10 μl of compact anti-HA agarose affinity beads with mixing at 4° C. overnight. After incubation, the beads were washed 4 times with 1 ml of HK buffer (100 mM KCl, 20 mM Hepes, pH 7.2), and the bound material was analyzed by immunoblotting.

Purification of GST-tagged ShhN²⁵⁻⁴⁹ and GST-tagged SURF4⁴⁹⁻⁶⁰ was performed as described previously (58). GST pull down assays were carried out with 10 μl of compact GSH beads bearing around 5 [symbol] g of GST-tagged ShhN²⁵⁻⁴⁹. The beads were incubated with 200 ml of 0.5 mg/ml of cell lysates from HEK293T cells transfected with HA-SURF4 in HKT buffer at pH 6.0 or 7.2 with mixing at 4° C. overnight. After incubation, the beads were washed three times with 500 ml of HKT buffer and twice with 500 ml of HK buffer, and the bound material was analyzed by immunoblotting.

Peptide binding assay was performed as described previously (59). Synthetic CW peptides (KRRHPKKC; SEQ ID NO: 6), CW^{MT} peptides (AAAHPAAC; SEQ ID NO: 9), SURF4⁴⁹⁻⁶¹ peptides (SEQRDYIDTTWNC; SEQ ID NO: 10), SURF4⁴⁹⁻⁶⁰ peptides (SEQRDYIDTTWN; SEQ ID NO: 12), or RRFR peptides (VRRFRYPERPC; SEQ ID NO: 11) were purchased from GenScript and coupled to thiopyridone-Sepharose 6B beads (Sigma-Aldrich) via the added C-terminal cysteine residue. For binding experiments, 2 [symbol] g purified GST-tagged SURF4⁴⁹⁻⁶⁰ or Shh²⁵⁻⁴⁹-GST was preincubated at 4° C. for 30 min in a total volume of 15 ml HK buffer. After incubation, 15 ml buffer containing around 5 [symbol] l beads containing 5 nmol of peptides was added to the reaction mixture for 1 h at 4° C. The beads were washed four times with 500 ml of HK buffer and analyzed by immunoblotting.

### Label-Free Quantitative Mass Spectrometry

Mass spectrometry were performed to identify the proteins involved in Shh secretion. After transfecting plasmids encoding Shh-HA or IGF2-HA into HEK293T cells, the immunoprecipitation was processed, and the bound proteins were analyzed by Coomassie Blue (Bio-Safe™ Coomassie-G250) staining. The protein gel was cut into small fragments and washed with 25 mM NH₄HCO₃/50% acetonitrile at room temperature for 15 min for three times. Then the gel fragments were shrunken by acetonitrile at room temperature for 15 min and dried by speed vacuum. The dried protein gel pieces were reduced by 0.1 M NH₄HCO₃ containing 10 mM TCEP at 55° C. for 45 min and alkylated by 0.1 M NH₄HCO₃ containing 55 mM Indoacetamide at room temperature in the dark for 45 min. After that, the gel pieces were washed by 0.1 M NH₄HCO₃ and repeat the steps of shrink and dry. Then the proteins were digested by 50 mM NH₄HCO₃ containing 20 ng/μl sequencing grade modified trypsin (Promega, number V511A) on ice for 45 min and incubated in 50 mM NH₄HCO₃ at 37° C. overnight. 25 mM NH₄HCO₃ and 60% acetonitrile containing 5% formic acid were used to exact the peptides respectively. Then the samples were dried with speed vacuum. The dried peptides were dissolved into 0.1% trifluoroacetic acid to remove the surfactant, desalted using pierce C18 spin column and dried by speed vacuum. Finally, the resulted peptides were analyzed by Mass Spectrometer. The tandem mass spectra were then subject to protein identification and label-free quantification by Proteome Discoverer. The proteins that were associated with the Shh or IGF2 were identified by comparing the peak intensity of the identified protein in the experimental group with the control group.

### In Vitro Vesicle Formation Assay

In vitro vesicular release assays were performed as described previously (59, 60). Briefly, Day 1 after transfection with plasmids encoding HA-tagged different version of ShhN, HEK293T cells grown in one 10-cm dish at around 90% confluence were permeabilized in 3 ml of ice-cold KOAc buffer containing 40 mg/ml digitonin on ice for 5 min, and the semi-intact cells were then sedimented by centrifugation at 300×g for 3 min at 4° C. The cell pellets were washed twice with 1 ml of KOAc buffer and resuspended in 100 ml of KOAc buffer. The budding assay was performed by incubating semi-intact cells (around 0.02 OD/reaction) with 2 mg/ml of rat liver cytosol in a 100 ml reaction mixture containing 200 mM GTP and an ATP regeneration system in the presence or absence of 0.5 mg of Sar1A (H79G). After incubation at 32° C. for 1 h, the reaction mixture was centrifuged at 14,000×g to remove cell debris and large membranes. The medium-speed supernatant was then centrifuged at 100,000×g to sediment small vesicles. The pellet fraction was then analyzed by immunoblotting. For density gradient flotation assays, the pellet fraction was resuspended in 100 ml of 35% OptiPrep and overlaid with 700 ml of 30% OptiPrep and 30 ml of KOAc buffer. The samples were centrifuged at 55,000 rpm in a TLS55 rotor in a Beckman ultracentrifuge for 2 hr at 4° C. After centrifugation, fractions were collected from the top to the bottom of the tube, and the top fraction was analyzed by SDS-PAGE and immunoblotting.

### Isothermal Titration Calorimetry (ITC)

We utilized a commercial isothermal titration calorimeter (PEAQ-ITC system, MicroCal, Malvern Panalytical, United Kingdom) to perform titration experiments. In these experiments, a 500 μM solution of CW peptide in a diluted binding buffer (10 mM PBS, 0.5 mM HEPES, 6.3 mM sorbitol, 1.75 mM KOAc, 25 μM Mg(OAc)₂, 25 μM BSA, 0.003% Triton, pH 7.2) was loaded into the syringe of the ITC instrument and a 50 μM solution of GST-SURF4 (49-60) in the same buffer was in the calorimetric cell. Similarly, GST was titrated with the CW peptide. During each ITC experiment, 19 injections of CW peptide into the calorimetric cell were carried out while duration of each injection was 4 s and the time between the injections was 150 s. The volume of each injection was 2 μL and the stirring speed was maintained at 750 rpm. Control experiments such as peptide to buffer and buffer to protein titrations were also done to account for non-specific interactions and heat of dilution. The binding constant (K_{d}) was derived by fitting the data with one set of site model using the MicroCal PEAQ-ITC analysis software.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—ER Export of ShhN Depends on its Cardin-Weintraub (CW) Motif

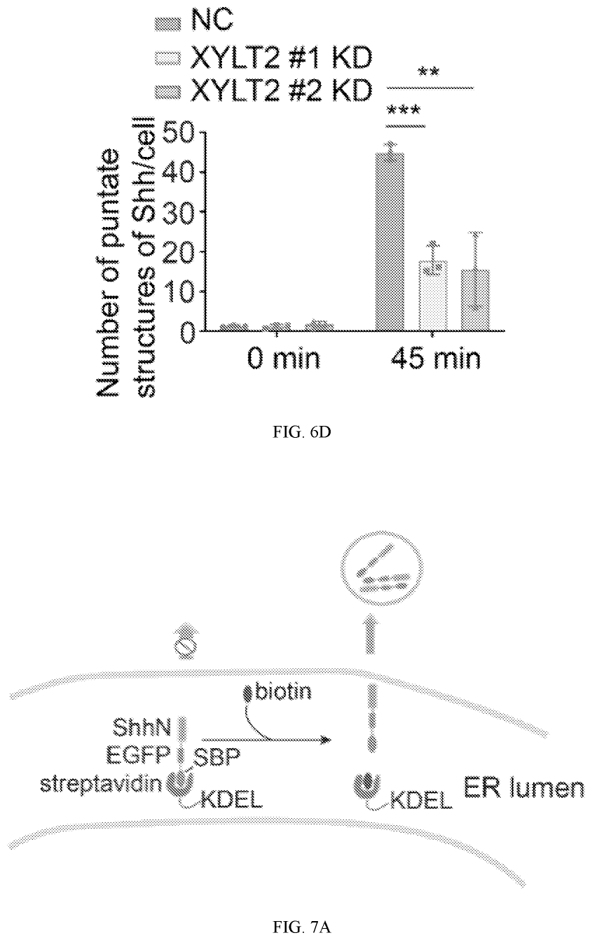

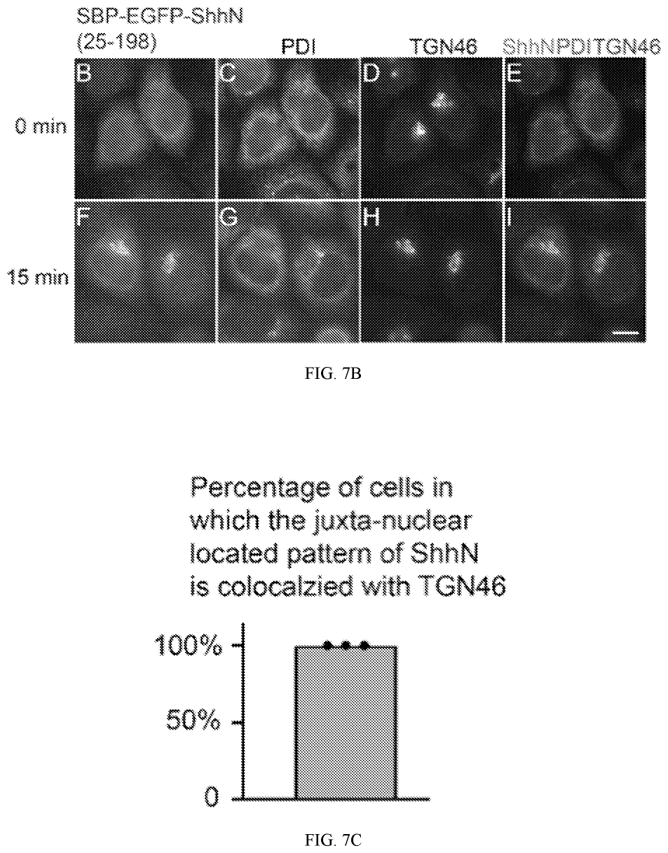

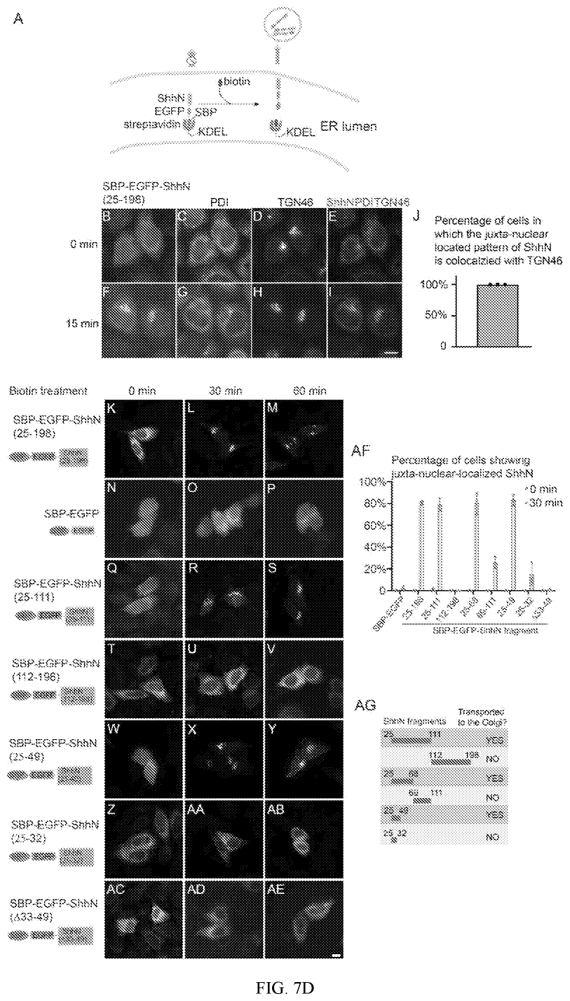

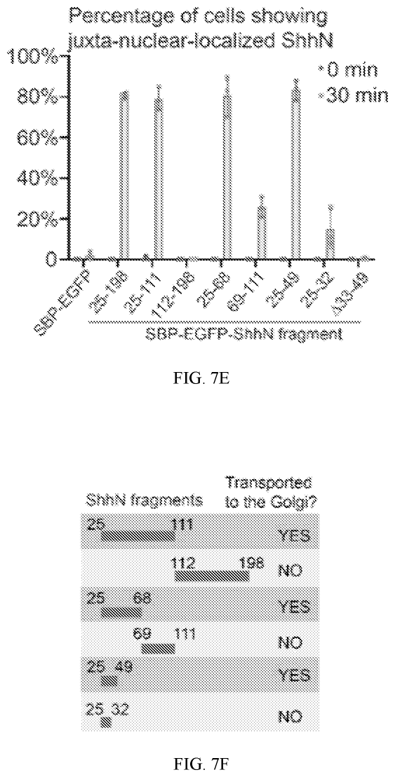

As a first step, in order to avoid potential complications from post-translational modification pathways, we examined the N-terminal fragment (ShhN) that lacks the cholesterol modification. A Retention Using Selective Hook (RUSH) transport assay (23) was performed to analyze surface delivery in a synchronized manner. In the RUSH assay, HeLa cells were transfected with plasmids encoding mouse N-terminal Shh fragment with the signal peptide removed (aa: 25-198) fused downstream of EGFP and the streptavidin binding peptide (SBP) that had an N-terminal signal peptide derived from IL-2 (SBP-EGFP-ShhN or SBP-EGFP-ShhN²⁵⁻¹⁹⁸, FIG. 7A). This plasmid also encodes streptavidin fused to a C-terminal ER retention signal (Lys-Asp-Glu-Leu; Str-KDEL). Due to the binding between streptavidin and SBP, SBP-EGFP-ShhN was retained in the ER, and colocalized with the ER marker protein disulfide isomerase (PDI) (FIG. 7B Panels B-E). When cells are incubated with biotin, SBP is released from streptavidin thereby releasing SBP-EGFP-ShhN from the ER (FIG. 7B Panels F-I, FIG. 7D Panels L-M). 15 min (FIG. 7B Panels F-I) or 30 min (FIG. 7D Panel L) after biotin treatment, SBP-EGFP-ShhN proteins localized at the juxta-nuclear area in the majority of cells. We counted 100 random cells showing a juxta-nuclear pattern of ShhN 15 min after biotin treatment in three independent experiments, finding that ShhN colocalized with the Golgi marker TGN46 in all of the cells (FIG. 7C). Thus, juxta-nuclear ShhN was considered to be located at the Golgi area in the following analyses. SBP-EGFP-ShhN proteins localized at the juxta-nuclear Golgi area in around 80% of cells after 30 min biotin treatment (FIG. 7D Panel L and quantification in FIG. 7E). A RUSH construct that did not contain the ShhN sequence (SBP-EGFP) was retained in the ER in over 95% of cells after biotin treatment (FIG. 7D Panels N-O and quantification in FIG. 7E). These results indicate that the RUSH assay is sufficiently robust to analyze the kinetics of ER-to-Golgi transport of ShhN. These results also suggest that ShhN contains motifs that drive efficient export of ShhN from the ER.

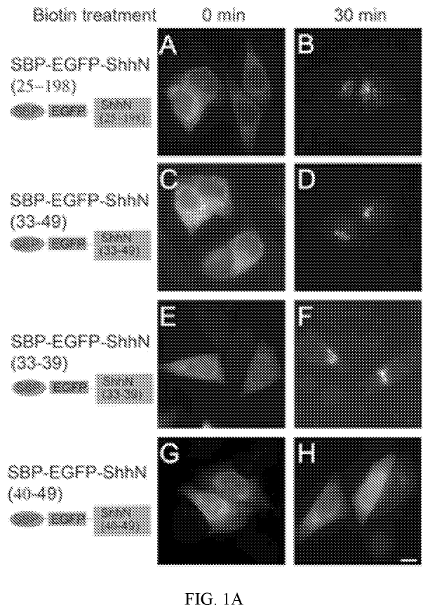

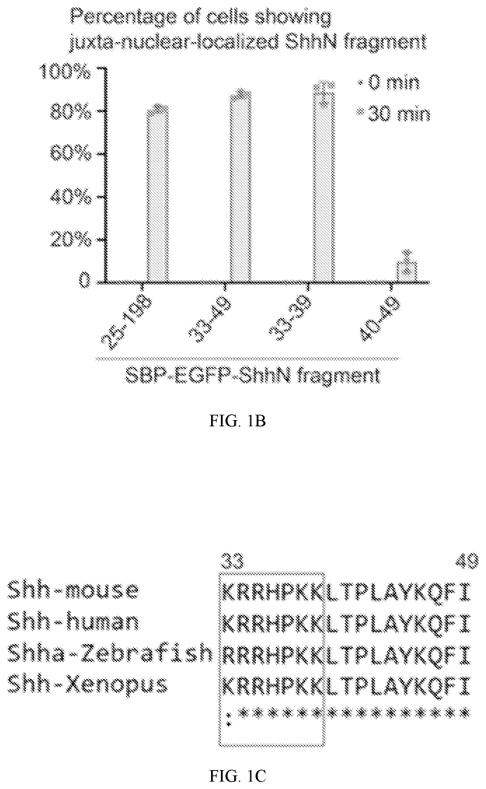

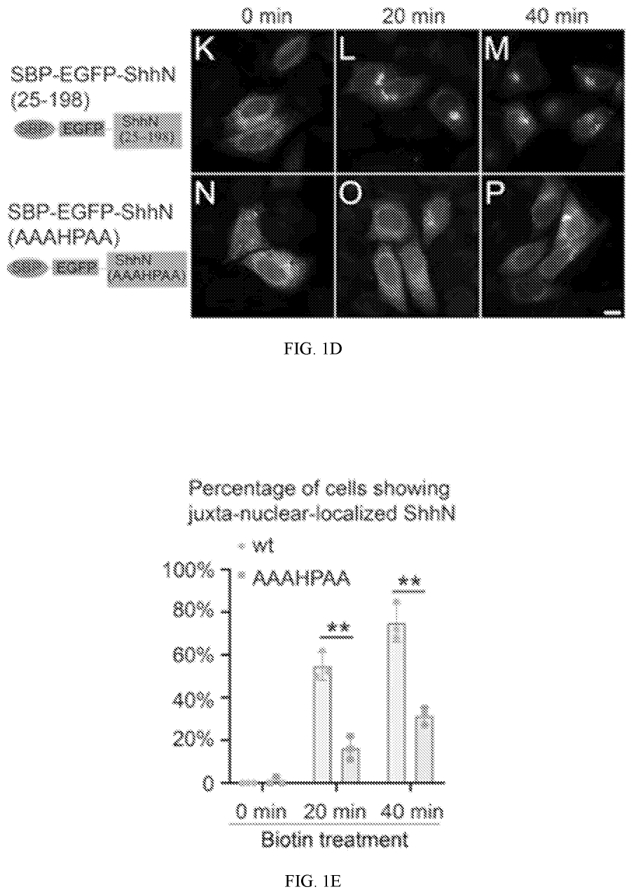

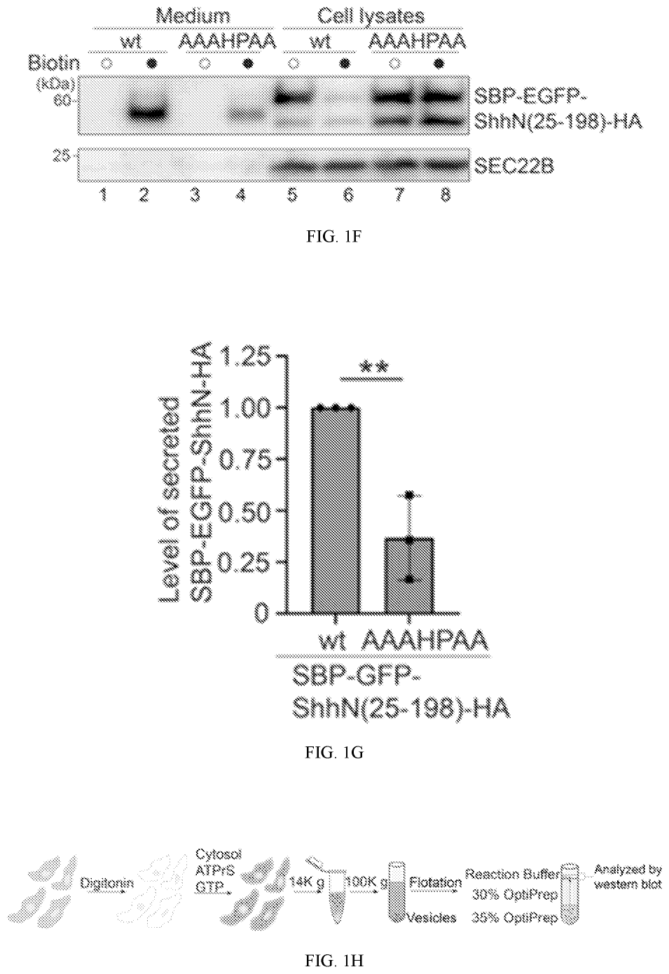

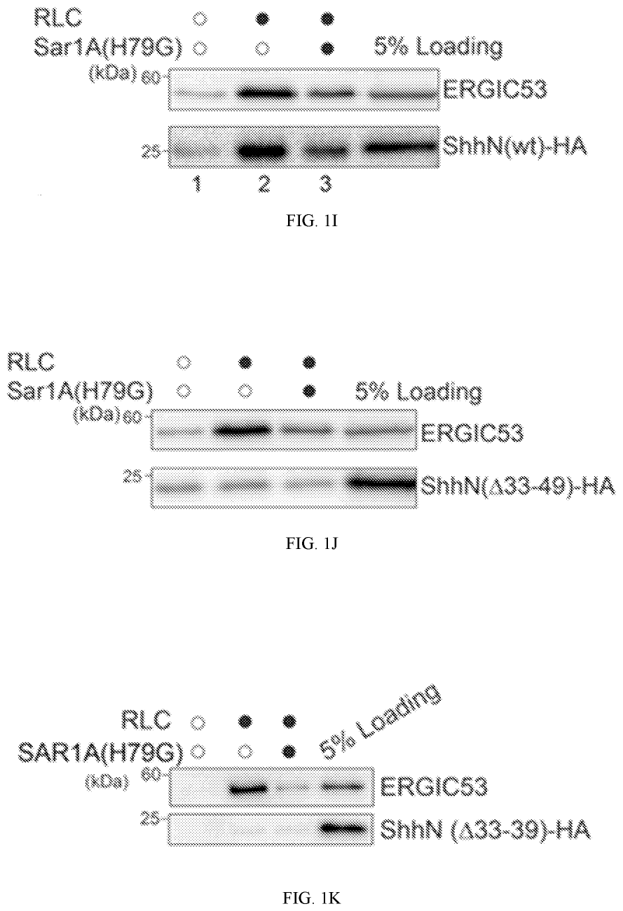

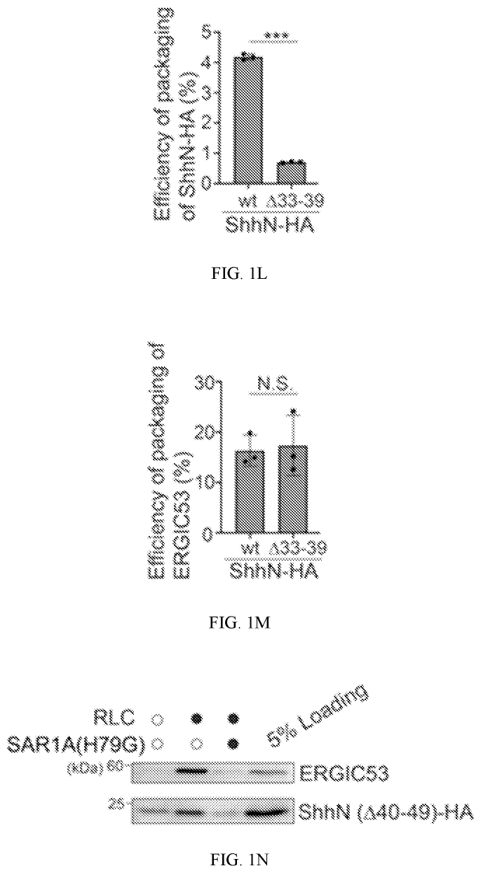

We next generated a series of the RUSH constructs containing truncated versions of Shh. We found that SBP-EGFP-ShhN²⁵⁻¹¹¹, SBP-EGFP-ShhN²⁵⁻⁶⁸, SBP-EGFP-ShhN²⁵⁻⁴⁹ were efficiently exported from the ER to the Golgi (FIG. 7D Panels Q-S, W-Y and FIG. 7E). In contrast, SBP-EGFP-ShhN¹¹²⁻¹⁹⁸, SBP-EGFP-ShhN⁶⁹⁻¹¹¹, SBP-EGFP-ShhN²⁵⁻³² were ER-retained (FIG. 7D Panels T-V, Z-AB and FIG. 7E), suggesting that residues 33-49 in ShhN are important for ER export (FIG. 7F). Indeed, deleting these residues in ShhN caused defects in ER-to-Golgi transport (FIG. 7D Panels AC-AE and FIG. 7E). Interestingly, we found that a RUSH construct composed of Shh residues 33-49 fused to SBP-EGFP (SBP-EGFP-Shh³³⁻⁴⁹) was delivered from the ER to the Golgi after biotin treatment with efficiency similar to that of SBP-EGFP-ShhN (FIG. 1A panels A-D and quantification in FIG. 1B), indicating that these residues are sufficient to allow SBP-EGFP to exit the ER. Further analysis indicated that residues 33-39 in mouse ShhN were sufficient for SBP-EGFP to exit the ER whereas residues 40-49 were not (FIG. 1A panels E-H and quantification in FIG. 1B).

The N-terminus of Shh is highly conserved. Sequence alignment of this region in mouse ShhN (amino acids 33-49) across species revealed a conserved KRRHPKK (SEQ ID NO: 8) or RRRHPKK (SEQ ID NO: 7) motif, termed the Cardin-Weintraub (CW) motif (B′B′B′XXB′B′, where B′ represents a basic amino acid (e.g., arginine or lysine), X represents any amino acid) (FIG. 1C). The CW motif is predicted to be a heparin binding domain that functions in protein-glycosaminoglycan (GAG) interactions (24). Alanine substitutions of the charged amino acids within this motif caused defects in ER-to-Golgi transport in the RUSH assay (FIG. 1D panels K-P and quantification in FIG. 1E). We therefore asked whether this motif is important for the release of ShhN to the extracellular milieu. To test this, we added an HA tag to the C-terminus of SBP-EGFP-ShhN and measured the efficiency of secretion by immunoblotting with antibodies against the HA tag. This analysis indicates that SBP-EGFP-ShhN-HA was secreted into the medium in a biotin-dependent manner (FIG. 1F, compare lanes 1 and 2). Mutating the charged amino acids among the CW motif significantly reduced the efficiency of secretion (FIG. 1F, compare lanes 2 and 4 and quantification in FIG. 1G). These results revealed that the CW motif plays an important role in exporting of ShhN from the ER, eventually to be secreted from the cells. We note that the mobility of SBP-EGFP-ShhN-HA in the medium was slightly different from that in cell lysates (FIG. 1F, compare lanes 2 and 5). The change in mobility may be caused by altered posttranslational modifications.

### Example 2—The CW Motif is Important for the Packaging of ShhN Into COPII Vesicles

To analyze whether the CW motif is important for the packaging of ShhN into COPII vesicles, we reconstituted vesicular release of ShhN in HEK293T cells (FIG. 1H). HEK293T cells transfected with 3xHA-tagged ShhN (ShhN-HA) were permeabilized by digitonin. After permeabilization, the semi-intact cells were washed with buffer to remove endogenous cytosolic proteins. Semi-intact cells were then incubated at 30° C. with rat liver cytosol (RLC), GTP and an ATP regeneration system (ATPrS) in the presence or absence of a GTP hydrolysis defective mutant form of Sar1A, Sar1A (H79G). The vesicles released were then isolated by centrifugation and analyzed by immunoblotting (FIG. 1H). SAR1A (H79G) inhibits COPII-dependent ER export (25) and abolishes the vesicular capture of standard COPII cargo proteins, SEC22B and ERGIC53 (26, 27). In contrast, SAR1A (H79G) does not affect the vesicular release of a trans Golgi network (TGN)-derived cargo protein, TGN46, but enhances membrane association of SEC23A/B (28). GTP hydrolysis of SAR1A allows the release of COPII from vesicle membranes to sustain efficient COPII vesicle formation. Based on these analyses, we consider the dependence on SAR1A (H79G) as indirect evidence for cargo packaging into COPII vesicles. ShhN-HA as well as a COPII cargo protein, ERGIC53, were efficiently packaged into transport vesicles in the presence of cytosol (FIG. 1I, compare lanes 1 and 2). The efficiencies of packaging of ShhN-HA and ERGIC53 into transport vesicles were greatly reduced when the vesicle formation assay was performed in the presence of Sar1A (H79G) (FIG. 1J, compare lanes 2 and 3), providing evidence that a major fraction of ShhN detected in the vesicle fraction was present in COPII vesicles. Deleting residues 33-49 (FIG. 1J) or deleting the CW motif (residues 33-39) in mouse ShhN caused a significant reduction in the efficiency of packaging of ShhN into vesicles (FIG. 1K and quantification in FIG. 1L), while the abundance of ERGIC53 in transport vesicles was unchanged (FIG. 1M). Deleting residues 40-49 caused no defects (FIG. 1M). These results indicate that the CW motif is important for the packaging of ShhN into COPII vesicles.

### Example 3—SURF4 Mediates Packaging of ShhN into Transport Vesicles and Regulates the ER-to-Golgi Trafficking and the Secretion of ShhN

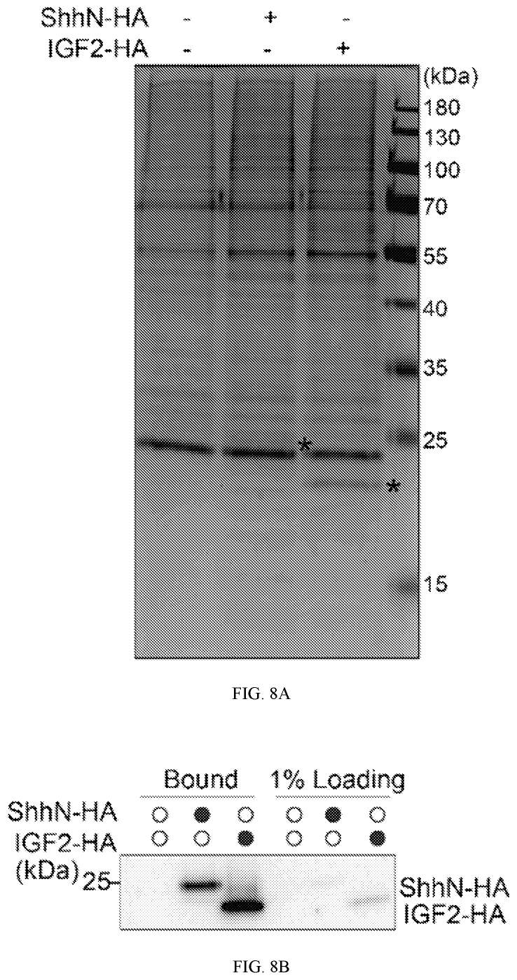

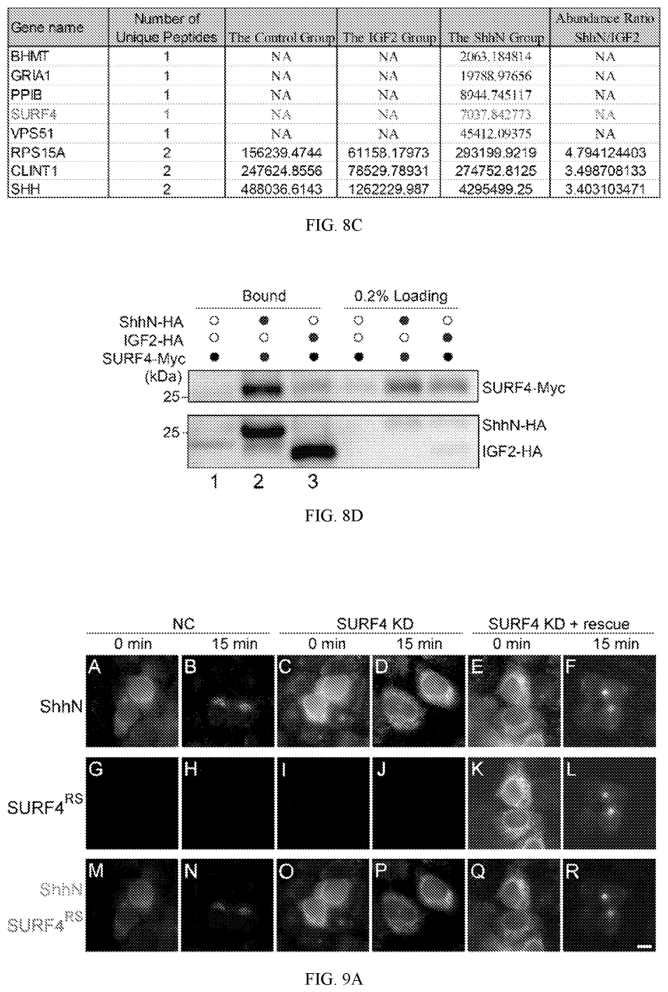

Soluble cargo proteins interact with the cytosolic COPII inner coat indirectly through transmembrane cargo receptors. To reveal cargo receptors that bind ShhN for packaging into transport vesicles, we performed immunoprecipitation experiments. Cell lysates from un-transfected HEK293T cells (the Control group) or cells transfected with plasmids encoding HA-tagged ShhN (the ShhN group) or HA-tagged insulin growth factor like-2 (IGF2, the IGF2 group) were incubated with beads conjugated with HA antibodies. The immobilized proteins were then eluted and analyzed by SDS-PAGE and Coomassie blue staining (FIG. 8A, asterisks indicate the position of ShhN-HA or IGF2-HA). Immunoblot analysis confirmed that IGF2-HA and ShhN-HA were efficiently immunoprecipitated (FIG. 8B). The eluted proteins were then trypsin digested and analyzed by mass spectrometry. We identified 5 proteins in the ShhN-HA group that were not identified in the other two groups (FIG. 8C): betaine-homocysteine S-methyltransferase (BHMT), glutamate receptor 1 (GRIA1), peptidyl-prolyl cis-trans isomerase B (PPIB), Surfeit locus protein 4 (SURF4) and vacuolar protein sorting-associated protein 51 homolog (VPS51). BHMT is a soluble protein and regulates homocysteine metabolism; GRIA1 belongs to a family of AMPA receptors; PPIB may assist folding of ShhN in the ER; VPS51 is involved in retrograde transport from early and late endosomes to the TGN and may regulate endosome-to-TGN trafficking of ShhN. SURF4 has been reported to mediate the ER export of soluble proteins, including lipoproteins and proprotein convertase subtilisin/kexin type 9 (PCSK9) (19-21) (FIG. 8C). Therefore, we hypothesized that SURF4 is the cargo receptor for Shh. To verify the mass spectrometry results, cell lysates from HEK293T cells transfected with plasmids encoding Myc-tagged SURF4 (SURF4-Myc) or cells co-transfected with plasmids encoding ShhN-HA and SURF4-Myc or cells co-transfected with IGF2-HA and SURF4-Myc were incubated with beads conjugated with HA antibodies. The immobilized proteins were then analyzed by immunoblot analysis. Co-immunoprecipitation results indicated that SURF4 interacted with ShhN more robustly than IGF2 (FIG. 8D, compare lanes 2 and 3), suggesting that SURF4 may function as a cargo receptor in the ER export of ShhN.

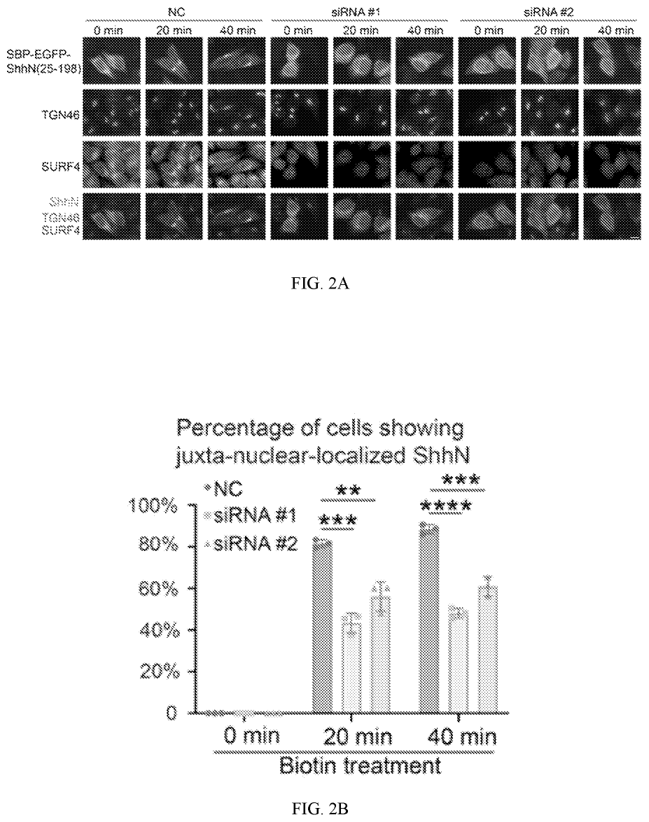

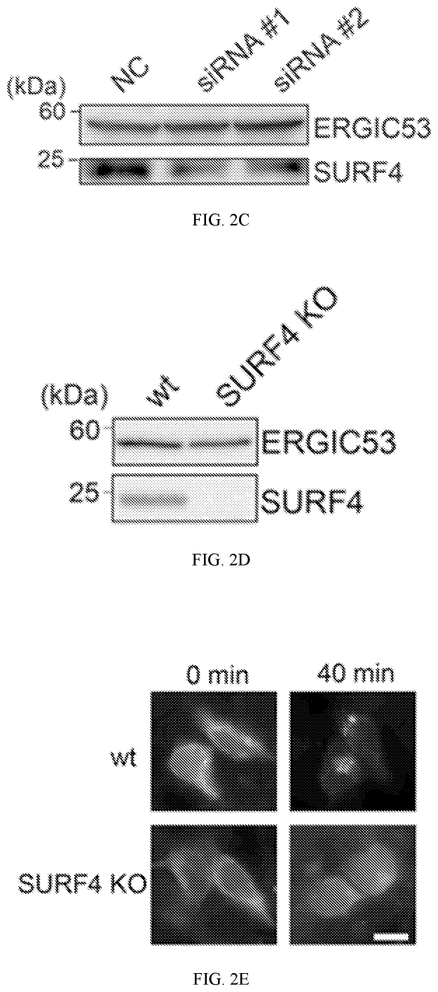

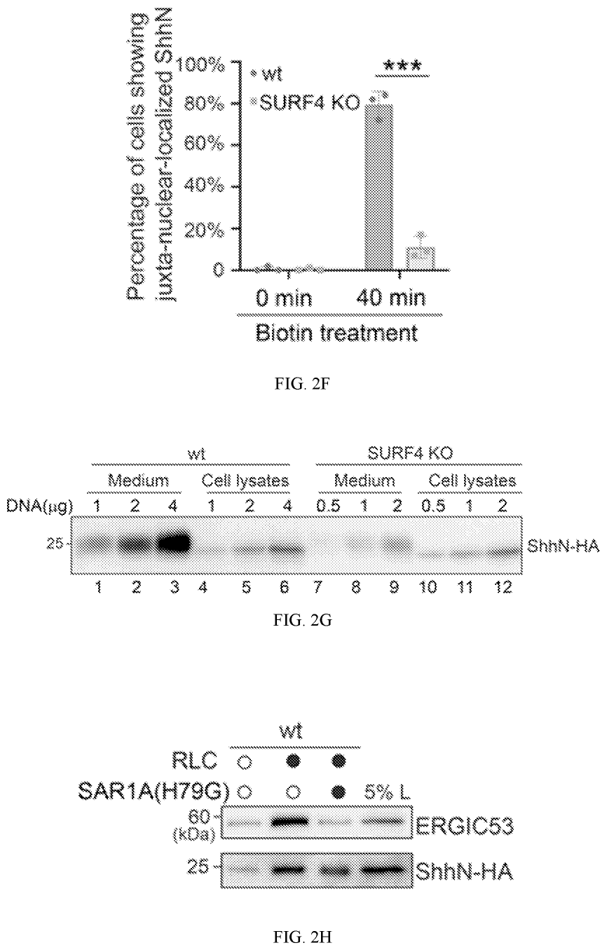

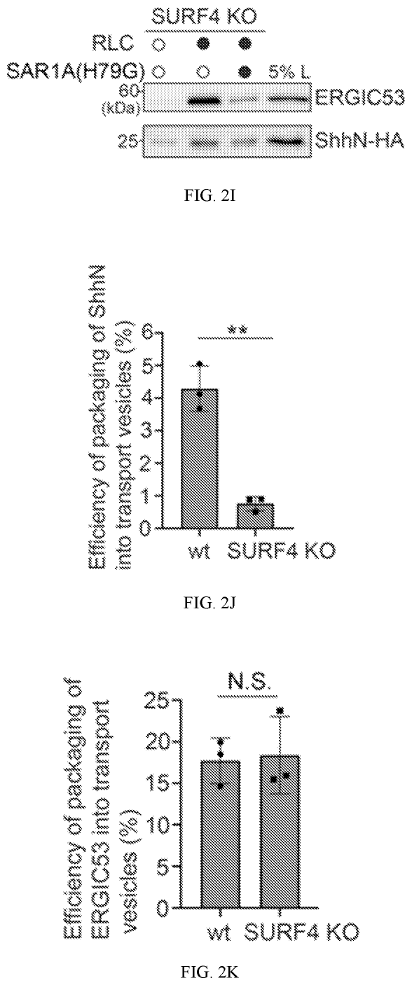

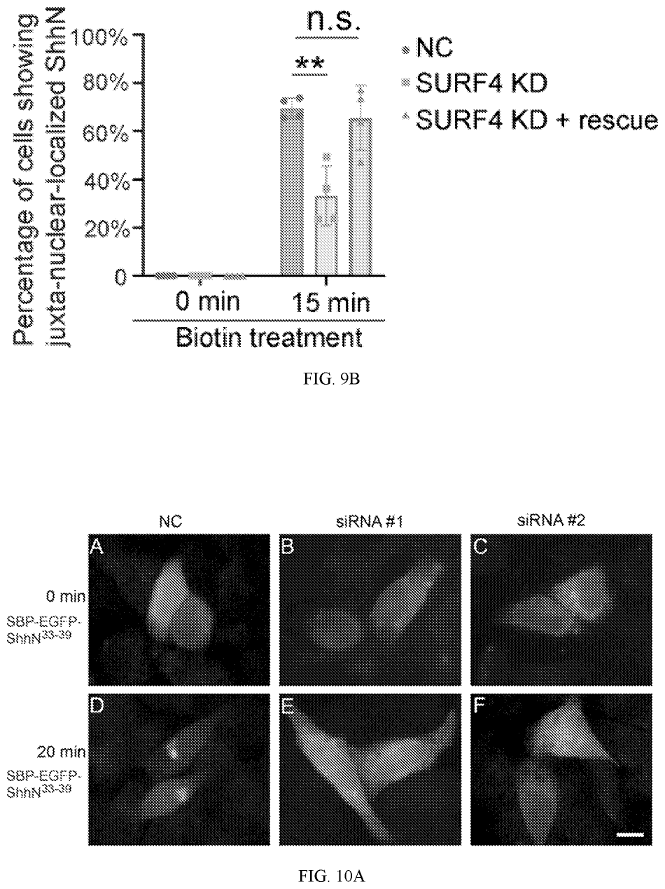

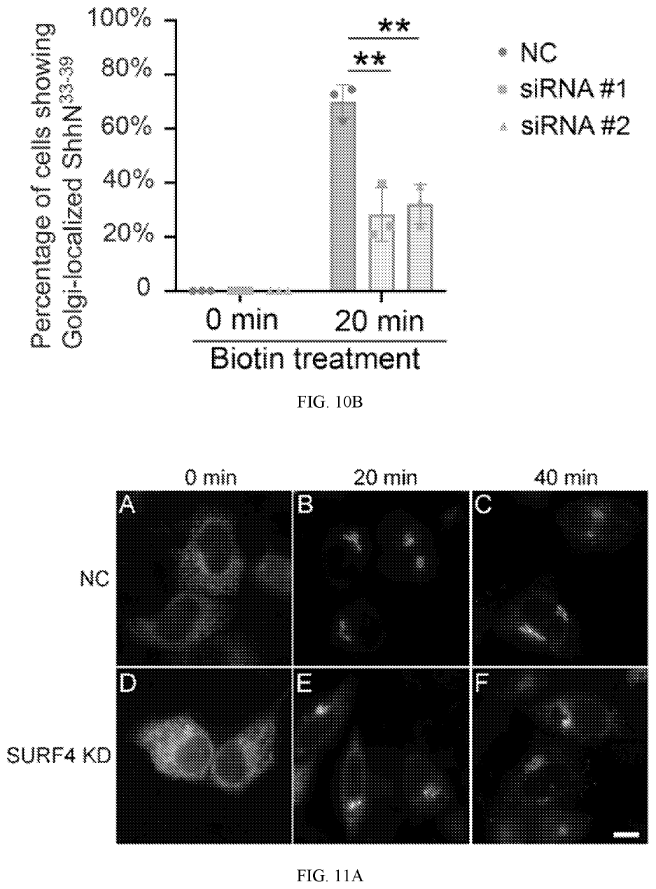

We next performed an siRNA knockdown experiment to reduce the expression of SURF4 and analyzed the impact on ER export of ShhN. Nearly all of the cells transfected with siRNA against SURF4 showed reduced SURF4 signal (FIG. 2A). Western blot analysis indicates that the level of SURF4 was greatly reduced in the knockdown cells (FIG. 2C). The nuclear staining pattern labelled by the SURF4 antibody may be caused by non-specific staining. We found that knockdown of SURF4 caused a kinetic delay in delivery of SBP-EGFP-ShhN to the Golgi (FIG. 2A and quantification in FIG. 2B). This defect was rescued by the expression of an siRNA-resistant construct of SURF4 (SURF4^{RS}-HA) (FIG. 9A and quantification in FIG. 9B). Knockdown of SURF4 also caused a kinetic delay in delivery of SBP-EGFP tagged mouseShhN (33-39) to the Golgi (FIG. 10A and quantification in FIG. 10B). We then generated HEK293Trex SURF4 knockout (KO) cells (FIG. 2D). An ER-to-Golgi trafficking defect was also detected in SURF4 KO cells (FIG. 2E and quantification in FIG. 2F). KO of SURF4 greatly reduced the secretion of ShhN-HA without the SBP-EGFP tag (FIG. 2G). Increasing the concentration of plasmids encoding ShhN-HA for transfection caused increased expression levels of ShhN-HA (FIG. 2G, compare lanes 4-6 and 10-12), and the secretion of ShhN-HA in SURF4 KO cells was greatly reduced in each condition (FIG. 2G, compares lanes 1-3 with lanes 7-9). Utilizing the in vitro vesicle formation assay, we found that ShhN-HA is packaged into transport vesicles in a cytosol-dependent manner in HEK293Trex cells and SARIA (H79G) reduced the efficiency of packaging (FIG. 2H). Knockout of SURF4 caused a significant reduction of the efficiency of packaging of ShhN into transport vesicles, while the efficiency of packaging of ERGIC53 in transport vesicles was unchanged (FIGS. 2H-2I and quantifications in FIGS. 2J-2K).

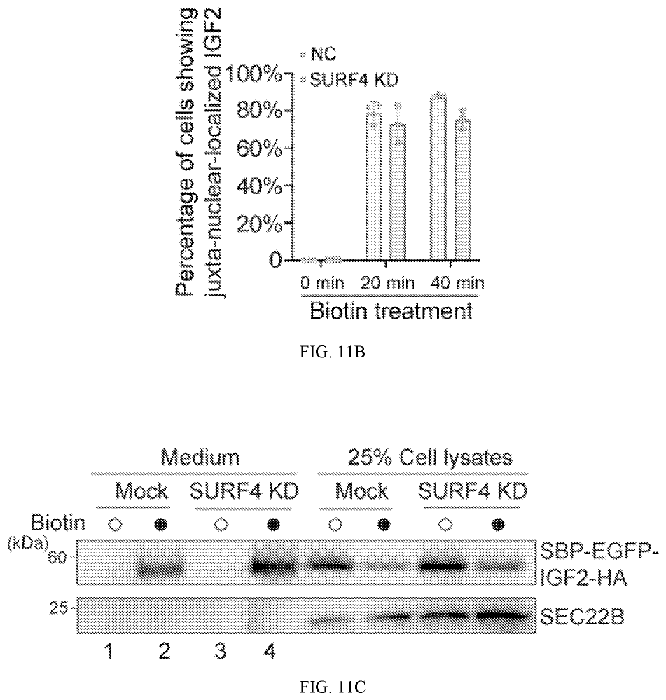

Since SURF4 binds more efficiently to ShhN than to IGF2 (FIG. 8D), we tested whether SURF4 regulates surface delivery of IGF2. We generated a RUSH construct of IGF2 (SBP-EGFP-IGF2). SBP-EGFP-IGF2 was delivered to the Golgi and secreted in a biotin-dependent manner (FIG. 11A panels A-C and FIG. 11C, compare lanes 1 and 2). ER-to-Golgi transport and secretion of SBP-EGFP-IGF2 was normal in the SURF4 knockdown cells (FIG. 11A panels D-F and quantifications in FIG. 11B, FIG. 11C, compare lanes 2 and 4). These results indicate that SURF4 functions as a cargo receptor to regulate the packaging of ShhN but not IGF2 into COPII vesicles to be delivered to the Golgi.

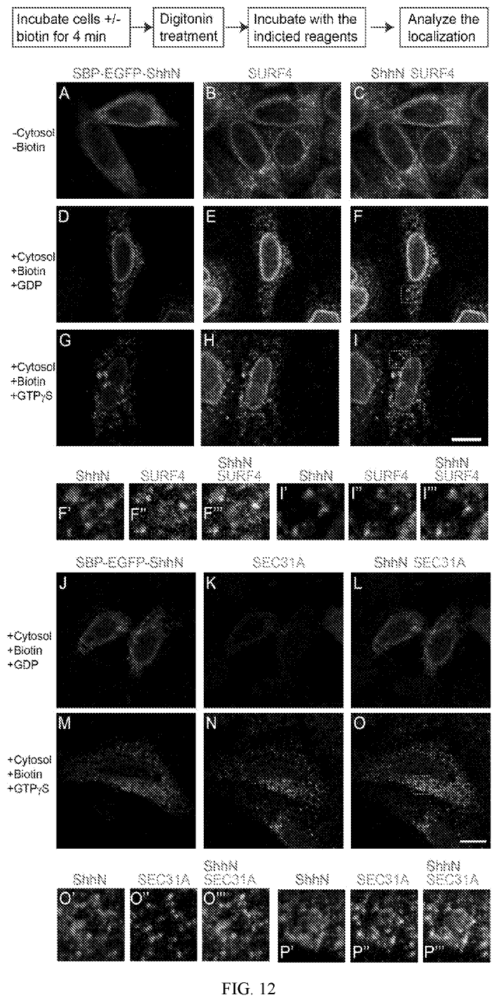

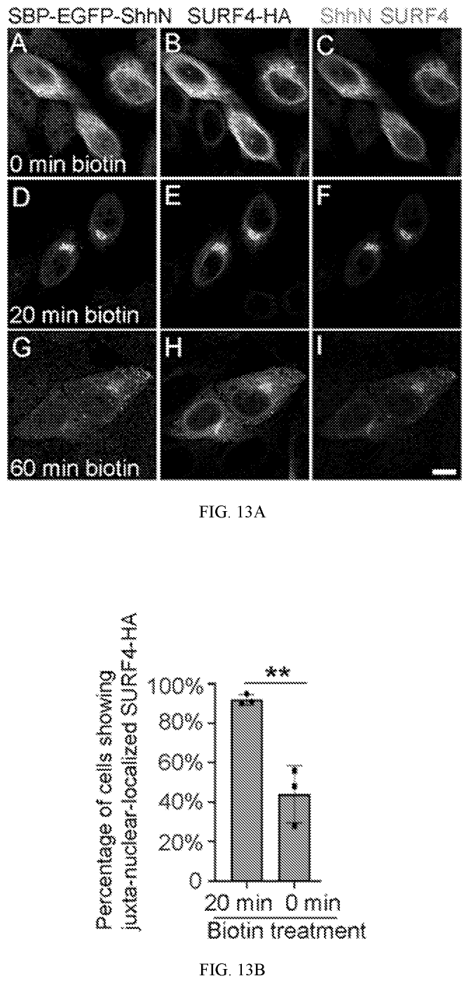

We next performed a permeabilized cell assay to analyze the colocalization between SURF4 and ShhN at early time points after biotin treatment. HeLa cells expressing SBP-EGFP-ShhN were incubated with biotin for 4 min, and permeabilized by digitonin. Subsequently, the semi-intact cells were washed to remove the endogenous cytosolic proteins and then incubated with rat liver cytosol in the presence of GDP or GTPγS. After such incubation, the COPII components are recruited to punctate structures in the cell periphery (28, 29) and Arf1 is recruited to the juxtanuclear Golgi area (30) in a GTP-dependent manner. We found that SBP-EGFP-ShhN showed an ER-located pattern after incubation without cytosol and biotin (FIG. 12 panels A-C). When the semi-intact cells were incubated with cytosol, biotin and GDP, SBP-EGFP-ShhN was partially located in peripheral punctate structures and partially located at the ER in the majority of cells (FIG. 12 panel D). When permeabilized cells were incubated with cytosol, biotin and GTPYS, SBP-EGFP-ShhN was partially located at punctate structures, with the ER pool of ShhN greatly diminished (FIG. 12 panel G). Although punctate structures of ShhN were detected in the cell periphery after incubation in the presence of GDP or GTPYS, we did not detect accumulation of ShhN at the juxtanuclear Golgi area 15 min after incubation with cytosol and nucleotides (FIG. 12 panels D and G). These analyses indicate that this assay locks the ER export process at the cargo sorting stage, providing a convenient way to accumulate cargo proteins at ERES and to analyze the colocalization between cargo proteins and their receptors.

Many of the punctate structures of SBP-EGFP-ShhN colocalized with SURF4 in the presence of either GDP or GTPγS (FIG. 12 panels D-I, magnified views in FIG. 12 panels F′-I′″), suggesting that SURF4 is associated with ShhN in a GTP-independent manner. SEC31A was recruited to the semi-intact cells in a GTP-dependent manner (FIG. 12 panels K, N), consistent with previous reports (28, 29). Many ShhN punctate structures partially overlapped with SEC31A in the presence of GTPγS (FIG. 12 panels M-O, magnified views in FIG. 12 panels O′-P′″), further suggesting stalled transport intermediates.

### Example 4—SURF4 Directly Interacts with the CW Motif on ShhN at the ER

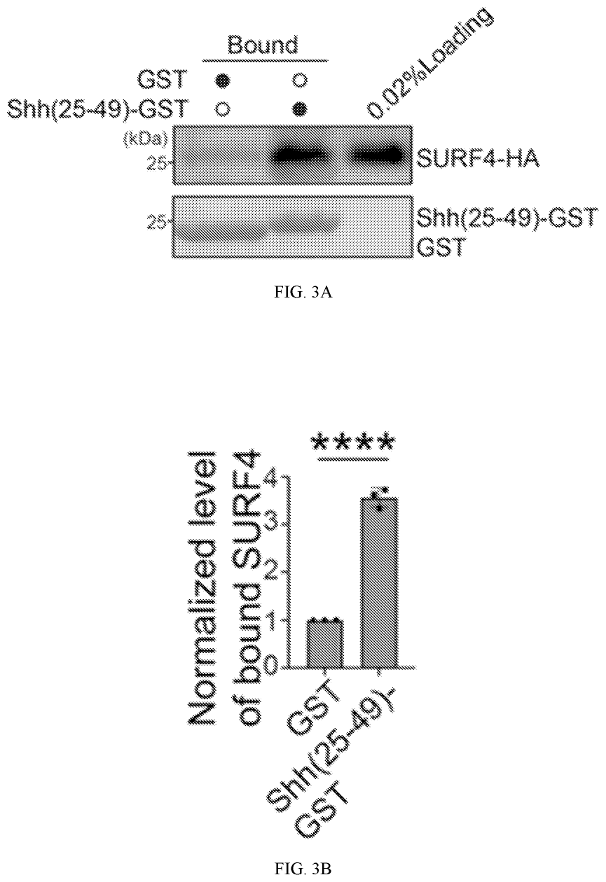

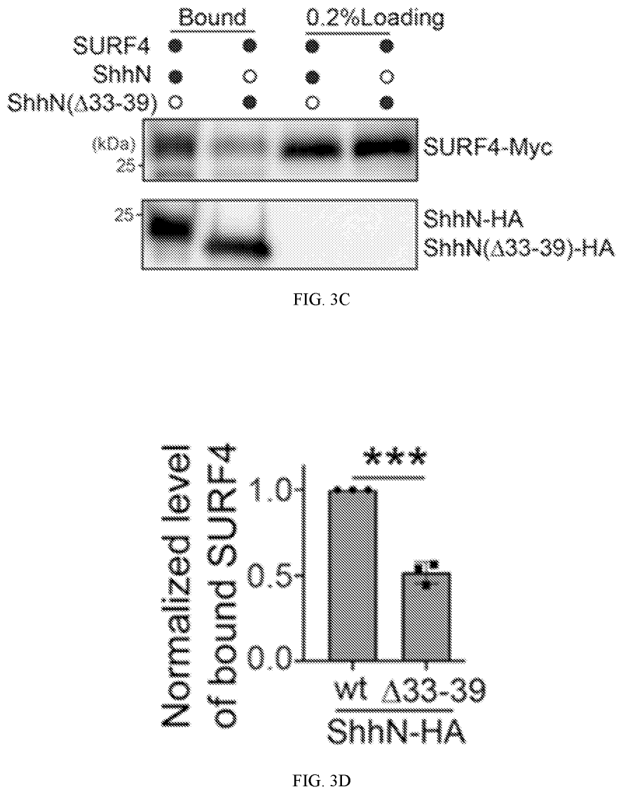

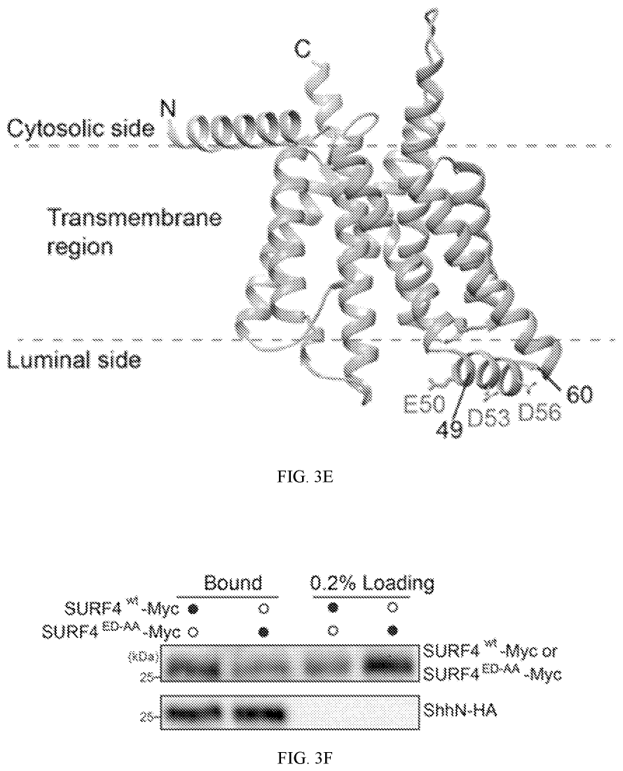

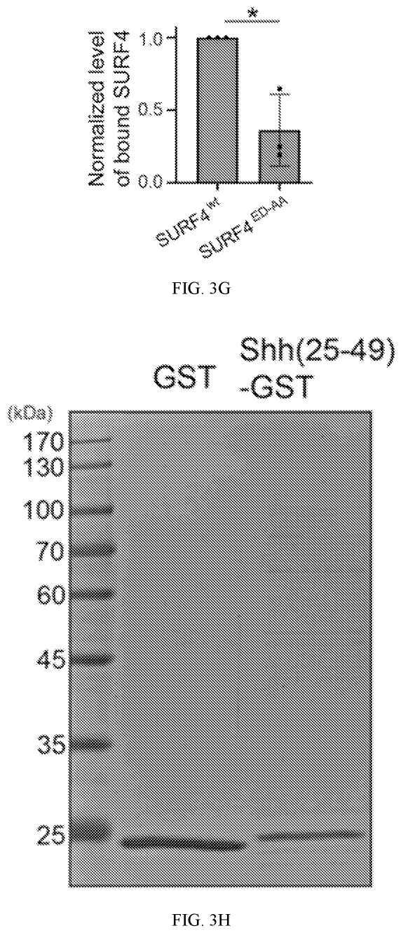

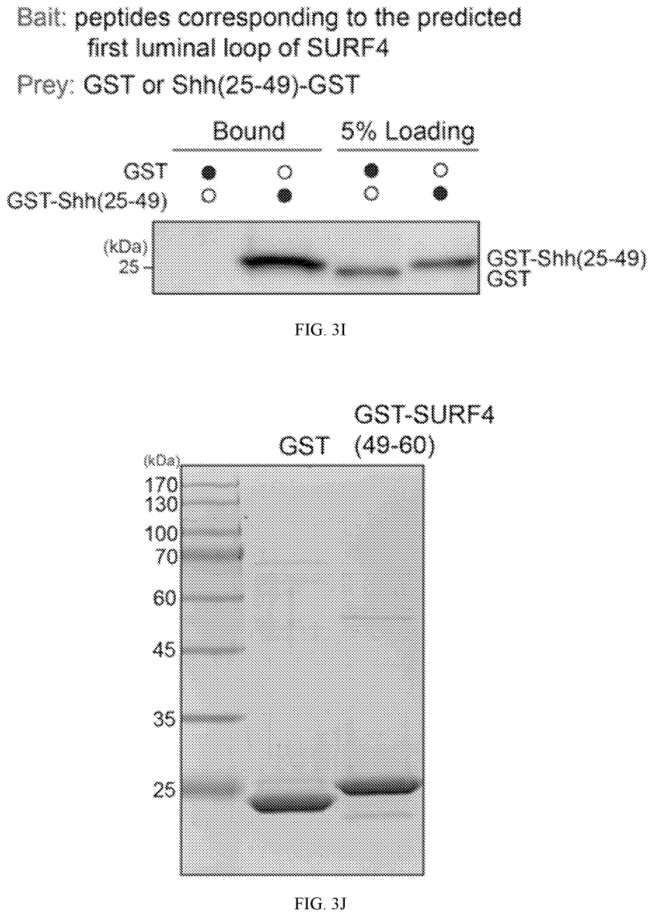

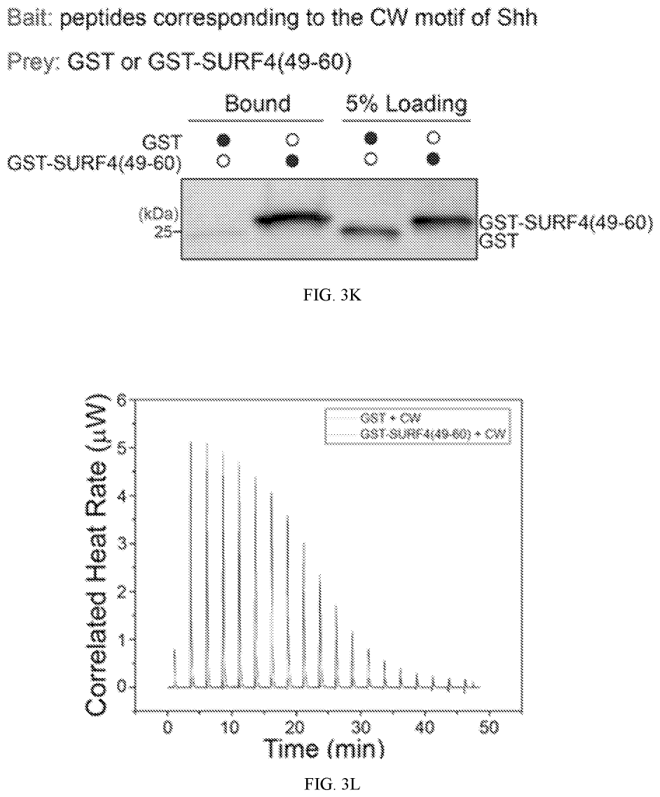

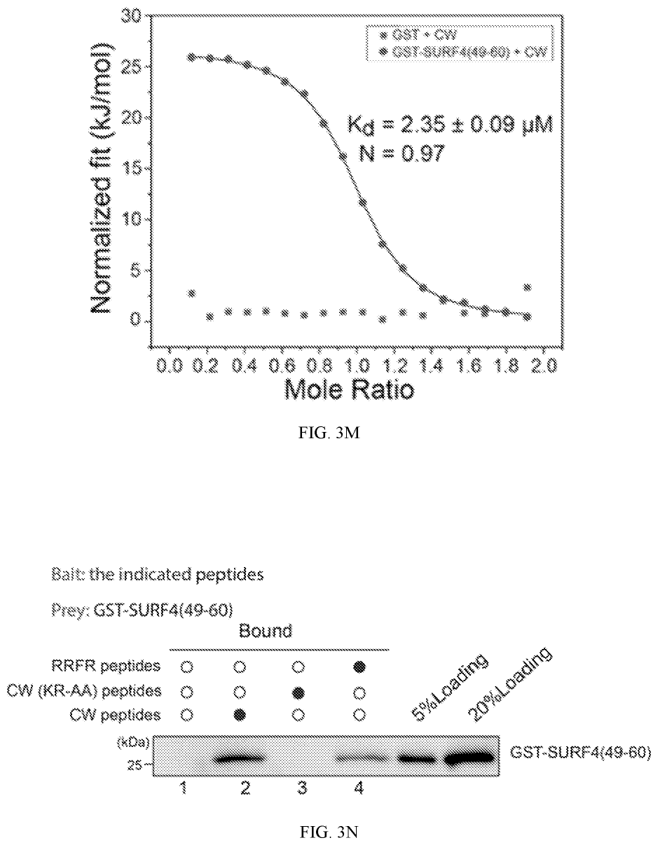

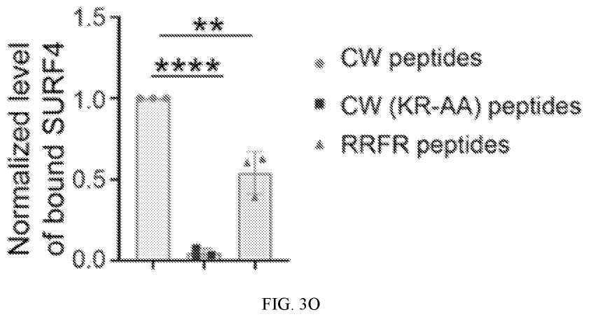

Since ER export of ShhN depends on its CW motif (FIGS. 1A-1M), we next tested whether this signal mediates interaction with SURF4. Purified GST-tagged ShhN²⁵⁻⁴⁹, which contains the CW motif in an N-terminal orientation, interacted with SURF4-HA from lysates of HEK293T cells overexpressing SURF4-HA. In contrast, GST alone recruited SURF4-HA poorly (FIG. 3A and quantification in FIG. 3B). To test whether the CW motif is important for the interaction between SURF4 and ShhN, we performed coimmunoprecipitation (co-IP) experiments using HEK293T cells co-transfected with plasmids encoding SURF4-Myc and ShhN-HA or HA-tagged CW motif-depleted mouse ShhN (ShhN^{Δ33-39}-HA). We found that the percentage of SURF4-Myc that bound to ShhN-HA was significantly higher than the percentage of SURF4-Myc that bound to mouse ShhN^{Δ33-39}-HA (FIG. 3C and quantification in FIG. 3D), suggesting that ShhN interacted with SURF4 through the CW motif.

The N- and C-termini of SURF4 are thought to be exposed to the cytosolic face of the ER, similar to the yeast homologue of SURF4, Erv29 (21, 31, 32). The structure of human SURF4 predicted by AlphaFold (33, 34) indicates that SURF4 contains 8 transmembrane helixes (FIG. 3E). The cytosolic N-terminus of SURF4 is predicted to form an amphipathic helix with the hydrophobic side contacting the cytosolic leaflet of the lipid bilayer. Interestingly, the first luminal loop of SURF4 (aa: 49-60) is predicted to form a helix with three negatively charged residues that point toward the lumen (residues E50, D53 and D56 highlighted in FIG. 3E). Co-IP analysis indicates that mutating these residues to alanine significantly reduced the interaction between SURF4-Myc and ShhN-HA (FIGS. 3F-3G).

To measure a direct interaction, we immobilized the first luminal loop of SURF4 (SEQRDYIDTTWNC; SEQ ID NO: 10, referred to as SURF4 luminal peptides) on beads and then performed pull down analysis using purified GST and Shh²⁵⁻⁴⁹-GST as prey (FIG. 3H). Strikingly, we found that Shh²⁵⁻⁴⁹-GST but not GST interacts with SURF4 luminal peptides (FIG. 3I). As an additional test, we performed pull down analysis using peptides corresponding to the CW motif (KRRHPKKC: SEQ ID NO: 6, referred to as CW peptides) as baits and purified GST-SURF4⁴⁹⁻⁶⁰ as prey (FIG. 3J). We found that GST-SURF4⁴⁹⁻⁶⁰ binds CW peptides, whereas GST binds weakly (FIG. 3K). Isothermal titration calorimetry (ITC)-based measurement indicates that GST-SURF4⁴⁹⁻⁶⁰ bound to CW peptides with a K_{d} of 2.35±0.09 μM, whereas no binding was detected between GST and CW peptides (FIGS. 3L-3M). Further analysis indicates that GST-SURF4⁴⁹⁻⁶⁰ bound more efficiently to the wild-type CW sequence than an alanine substituted mutant (AAAHPAAC: SEQ ID NO: 9, referred to as CW(KR-AA) peptides) (FIG. 3N, compare lanes 2 and 3, and quantification in FIG. 3O). These results indicate that the CW motif directly interacts with the first luminal domain of SURF4 through electrostatic interactions. We then immobilized peptides corresponding to the first intracellular loop of Frizzled6 (VRRFRYPERPC; SEQ ID NO: 11, referred to as RRFR) (27) on beads. Although RRFR peptides are also positively charged, the level of GST-SURF4⁴⁹⁻⁶⁰ bound to RRFR peptides was significantly lower than that bound to the CW peptides (FIG. 3N, compare lanes 2 and 4, and quantification in FIG. 3O).

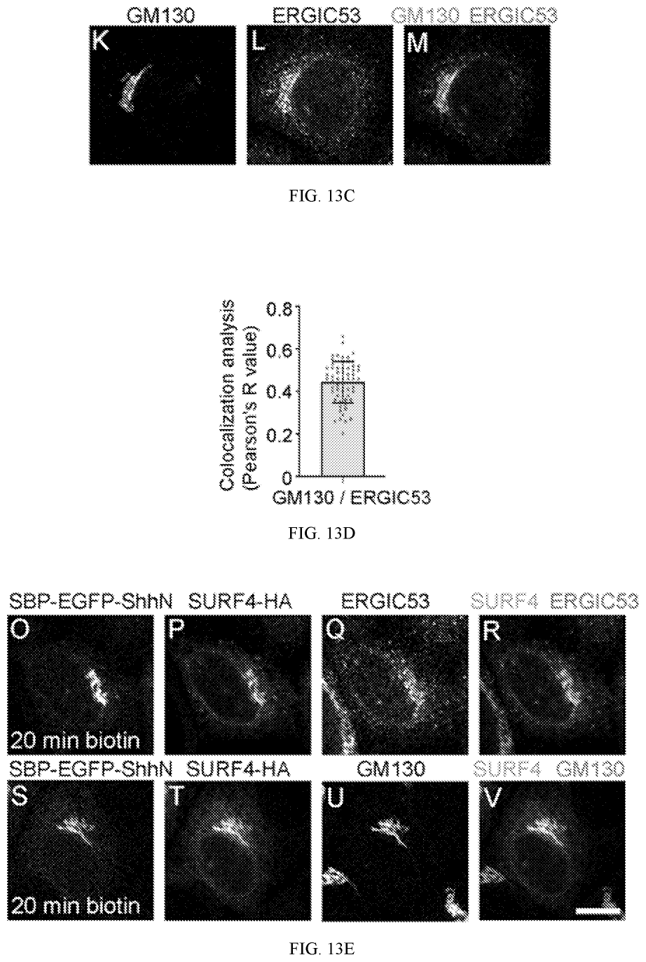

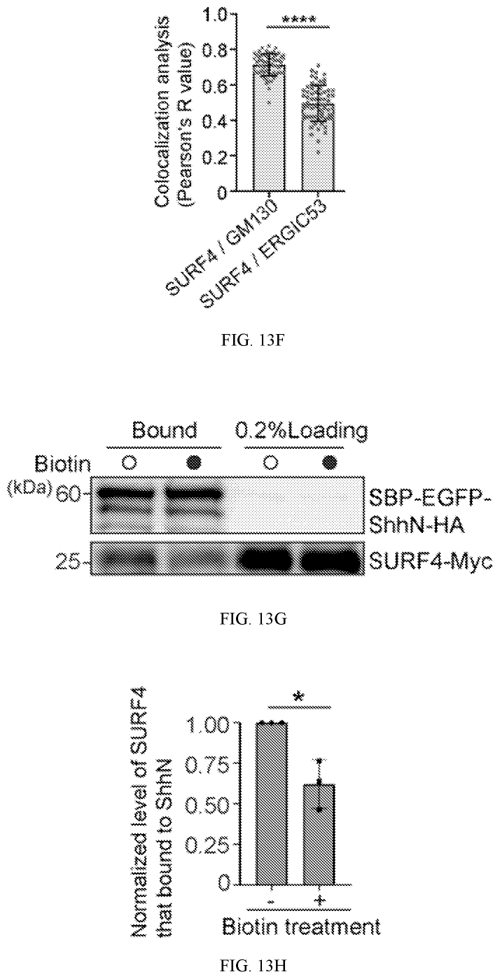

SURF4 is shown to localize to the ER, ERES and ER-Golgi intermediate compartment (ERGIC) (20, 22). Mutations in the COPI-binding motif of SURF4 or expression of the GTPase defective mutant form of Arf1, Arf1(Q71L), accumulate SURF4 at the Golgi, suggesting that SURF4 cycles between the ER and the Golgi (21). When SURF4-HA was co-expressed with SBP-EGFP-ShhN, it was located at the ER in the absence of biotin in ˜60% of the SURF4 and ShhN co-expressing cells (FIG. 13A panels A-C and quantification in FIG. 13B). 20 min after biotin treatment, SBP-EGFP-ShhN was located at the juxta-nuclear Golgi area (FIG. 13A panels D-F). Interestingly, SURF4-HA was co-localized with SBP-EGFP-ShhN at the juxta-nuclear Golgi area 20 min after biotin treatment in ˜90% of the SURF4 and ShhN co-expressing cells (FIG. 13A panels D-F and quantification in FIG. 13B). Quantification indicates that the fraction of cells showing juxta-nuclear SURF4-HA was significantly increased 20 min after biotin treatment in the co-expressing cells (FIG. 13B). 60 min after biotin treatment, SBP-EGFP-ShhN was exported out of the Golgi and was located to some intracellular punctate structures in the cytoplasm and near the cell surface (FIG. 13A panel G). In contract, SURF4-HA was localized in the ER and the Golgi area at this time point (FIG. 13A panel H). These analyses indicate that SURF4 traffics together with SBP-EGFP-ShhN from the ER to the Golgi, then SURF4 is retrieved to the ER while ShhN travels to the cell surface.

As an additional experiment to test whether SURF4 traffics together with ShhN to the Golgi, we compared the localization of SURF4 20 min after biotin treatment with the localization of a cis-Golgi marker, GM130 or with the localization of an ERGIC marker, ERGIC53, in HeLa cells. GM130 showed a juxta-nuclear localization pattern (FIG. 13C panel K). ERGIC53 showed a punctate localization pattern in the cell periphery and also a juxta-nuclear localization pattern (FIG. 13C panel L). The punctate pattern of ERGIC53 is adjacent to the ERES (35) and the juxta-nuclear pattern of ERGIC53 partially colocalized with GM130 (FIG. 13C panel M). The majority of SURF4-HA was located at the juxta-nuclear area 20 min after biotin treatment (FIG. 13E panels P and T). The juxta-nuclear-located SURF4 overlapped more with GM130 than with ERGIC53 (FIG. 13E panels R and V). We quantified the colocalization between ERGIC53 and GM130 in the juxtanuclear area labelled by GM130 (FIG. 13D). We also quantified the colocalization between SURF4-HA and GM130 or ERGIC53 in the juxtanuclear area labelled by SBP-EGFP-ShhN (FIG. 13F). The Pearson correlation coefficient (Pearson's R value) was calculated as an indicator of the colocalization. This quantification indicates that the colocalization between SURF4-HA and GM130 was significantly higher than that between SURF4-HA and ERGIC53 (FIG. 13F, R values of 0.714 and 0.496, respectively). These analyses suggest that SURF4 and ShhN traffic together to the cis Golgi 20 min after biotin treatment.

We performed co-IP experiments using HEK293T cells co-transfected with plasmids encoding SURF4-Myc and Str-KDEL_SBP-EGFP-ShhN-HA with or without biotin treatment. Under the no-biotin condition, the majority of SURF4 and ShhN locate to the ER (FIG. 13A Panels A-C), and robustly co-IP (FIGS. 13G-13H). Under conditions of biotin treatment, combined with incubation at 20° C. to block cargo export from the TGN, the majority of ShhN and SURF4 showed a Golgi-localized pattern (FIG. 13A panels D-F) and co-IP was reduced (FIGS. 13G-13H). Together, these data suggest that ShhN and SURF4 interact with each other at the ER and separate from each other after entering the Golgi.

### Example 5—Proteoglycans (PGS) Regulate Export of ShhN out of the TGN

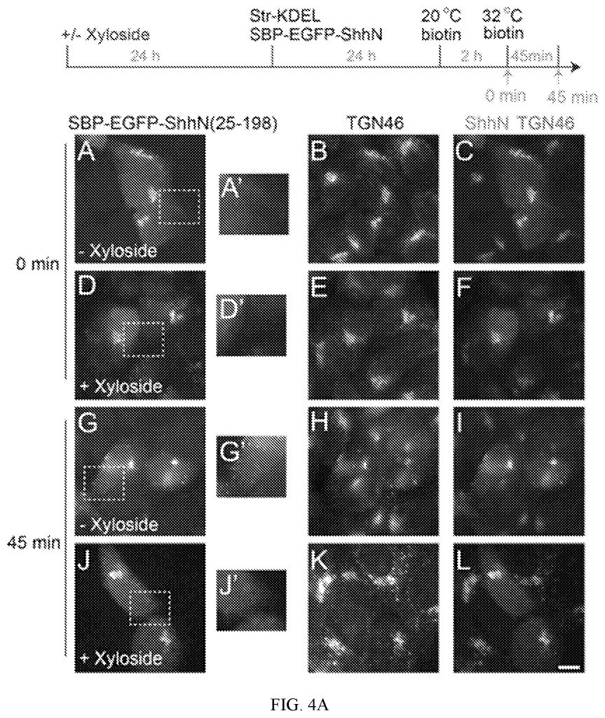

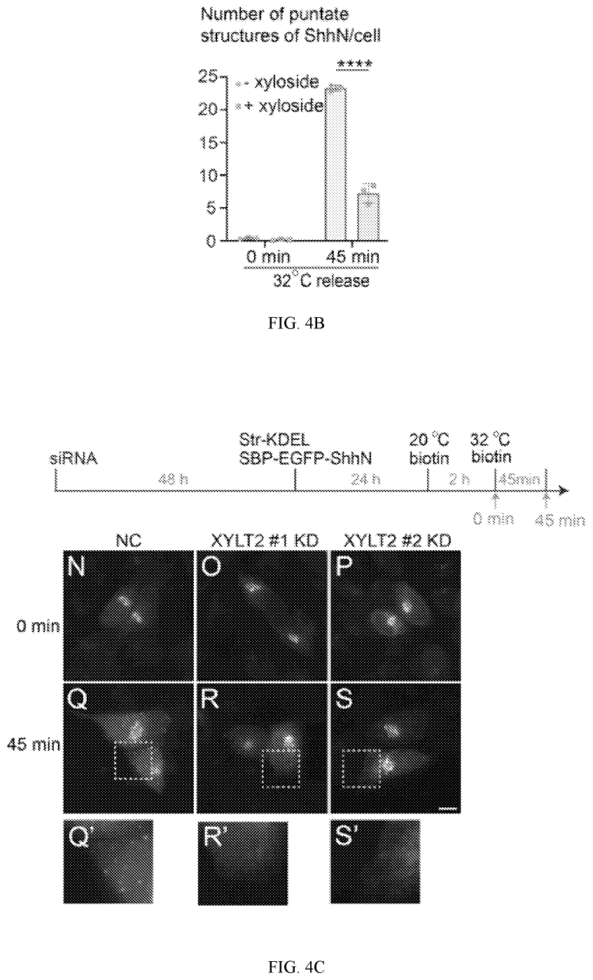

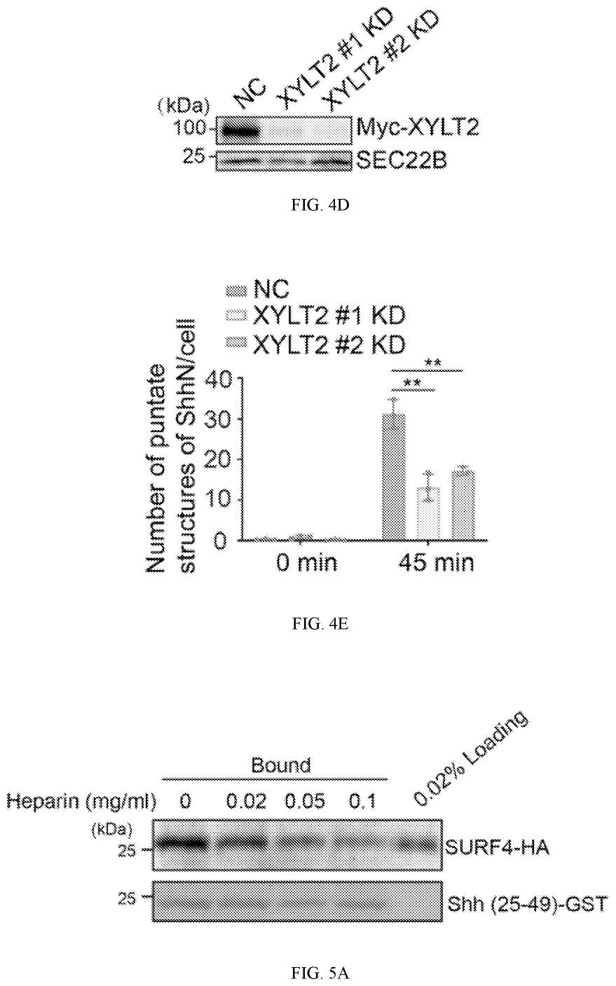

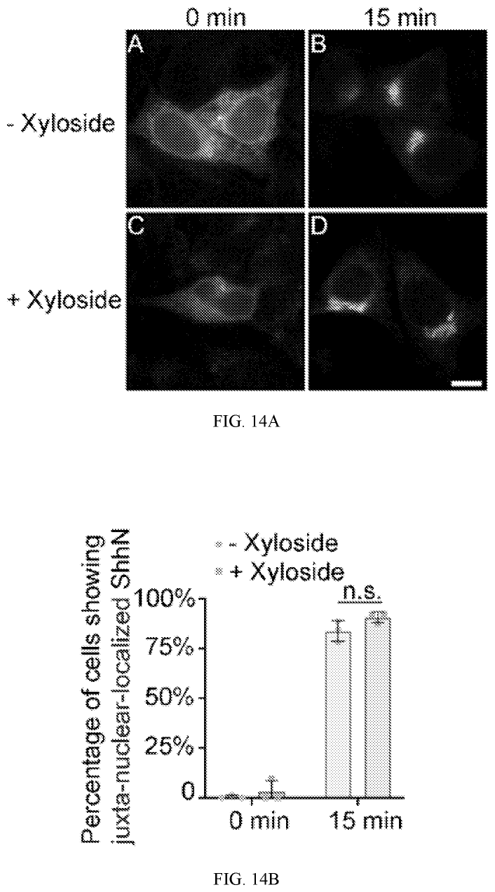

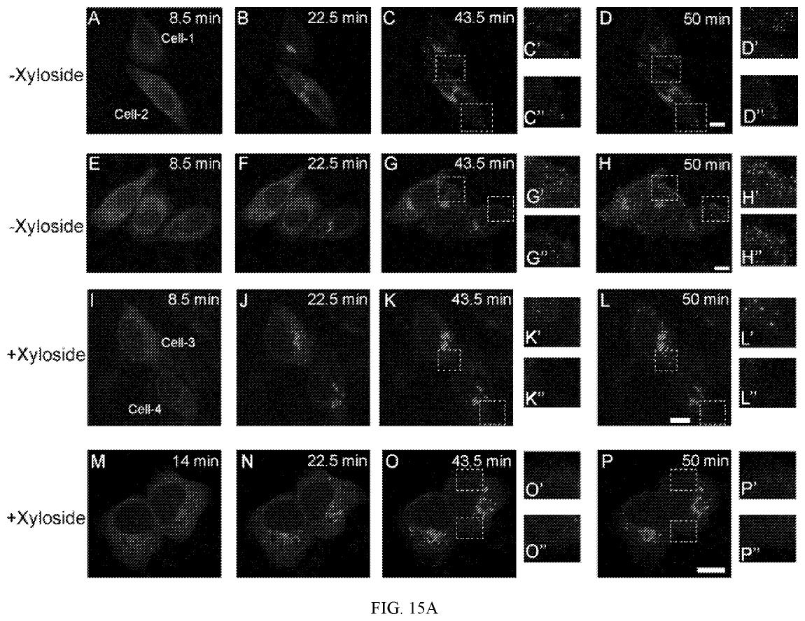

PGs are important for TGN export of soluble cargo proteins including the soluble enzyme lipoprotein lipase (LPL) (36). Therefore, we tested whether PGs regulate TGN export of ShhN. We treated the cells with xyloside, which inhibits the attachment of GAGs during PG maturation. Treatment with xyloside did not cause defects in ER-to-Golgi transport of SBP-EGFP-ShhN (FIGS. 14A-14B). We then analyzed the kinetics of TGN export of ShhN using the RUSH assay. HeLa cells expressing Str-KDEL and SBP-EGFP-ShhN were incubated at 20° C. in the presence of biotin to accumulate cargo in the TGN, then shifted to 32° C. to release cargo. After the 20° C. incubation, SBP-EGFP-ShhN accumulated at the juxta-nuclear Golgi area with no detectable punctate structures in the cytoplasm (FIG. 4A panels A-F, and magnified views in panels A′ and D′). 45 min after incubation at 32° C., SBP-EGFP-ShhN in the majority of cells showed a punctate localization pattern (FIG. 4A panels G-I, and magnified view in panel 4G′). We hypothesize that these punctate structures are TGN-derived transport vesicles enriched with SBP-EGFP-ShhN. The average number of punctate structures containing SBP-EGFP-ShhN in each expressing cell 45 min after incubation at 32° C. was significantly decreased after xyloside treatment (FIG. 4A panels J-L, magnified views in panel J′, and quantification in FIG. 4B).

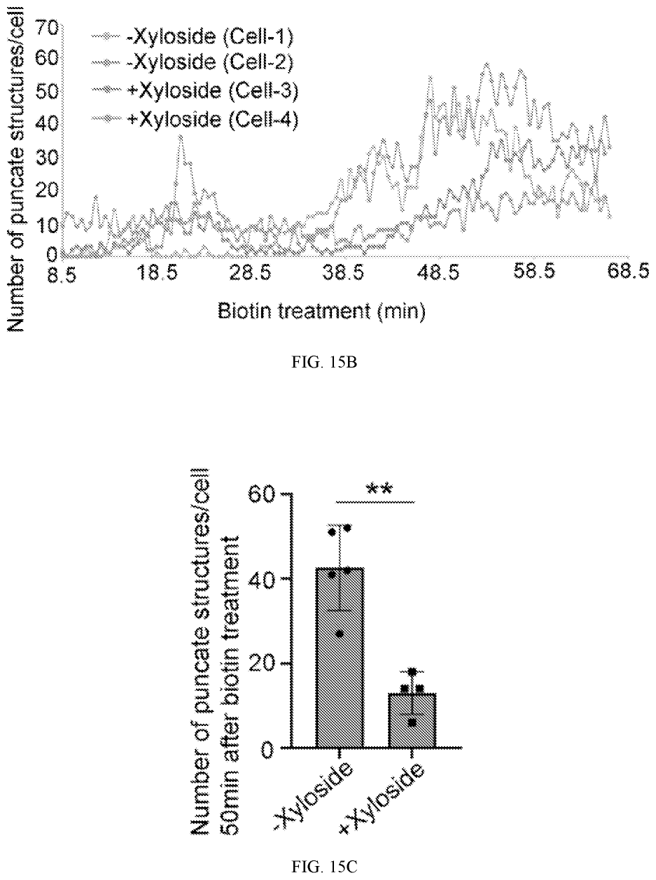

We next performed a live imaging analysis to visualize the surface delivery of SBP-EGFP-ShhN. We found that SBP-EGFP-ShhN was delivered to the juxta-nuclear Golgi area after biotin treatment (FIG. 15A panels A-H). We observed punctate structures of SBP-EGFP-ShhN in the cytoplasm during post-Golgi trafficking of SBP-EGFP-ShhN, and the majority of these punctate structures showed a clear mobility towards the cell surface (FIG. 15A panels C-D and G-H, and magnified views in panels C′-D″ and G′-H″). These punctate structures are post-Golgi vesicles. Xyloside treatment did not block the ER-to-Golgi transport of SBP-EGFP-ShhN (FIG. 15A panels I-J and M-N) but the number of punctate structures during post-Golgi trafficking was greatly reduced compared to the cells without drug treatment (FIG. 15A panels K-L and O-P, and magnified views in panels K′-L″ and O′-P″, quantification in FIGS. 15B-15C). We did not observe any apparent movement of the punctate structures toward the cell surface in the drug treated cells. The live imaging analyses were consistent with our analyses using fixed cells, indicating that synthesis of PGs regulates export of ShhN out of the TGN.

As an additional experiment to test the effect of PG synthesis on TGN export of ShhN, we knocked down expression of xylosyltransferase 2 (XYLT2), which catalyzes the attachment of GAG chains to PG core proteins, using two different siRNAs (FIG. 4D). We then performed a temperature shift experiment, in which cells were incubated at 20° C. in the presence of biotin to accumulate cargo in the TGN, then shifted to 32° C. to release cargo. We quantified the number of punctate structures of SBP-EGFP-ShhN 45 min after incubation at 32° C. The average number of punctate structures of SBP-EGFP-ShhN in each expressing cell was significantly decreased in cells transfected with either siRNA against XYLT2 (FIG. 4C panels N-S, magnified views in panels Q′-S′, and quantification in FIG. 4E), suggesting a defect in export of SBP-EGFP-ShhN out of the TGN in XYLT2 knockdown cells.

### Example 6—PGS Compete With SURF4 to Bind ShhN and Facilitate Trafficking of ShhN Through the Golgi

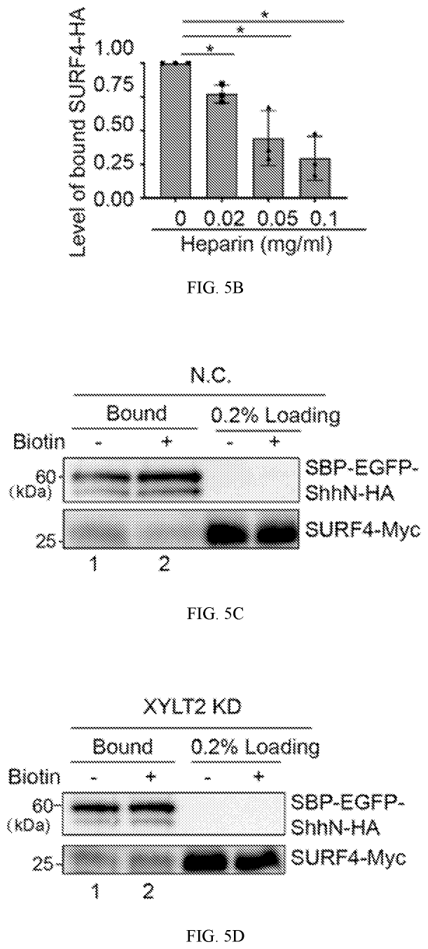

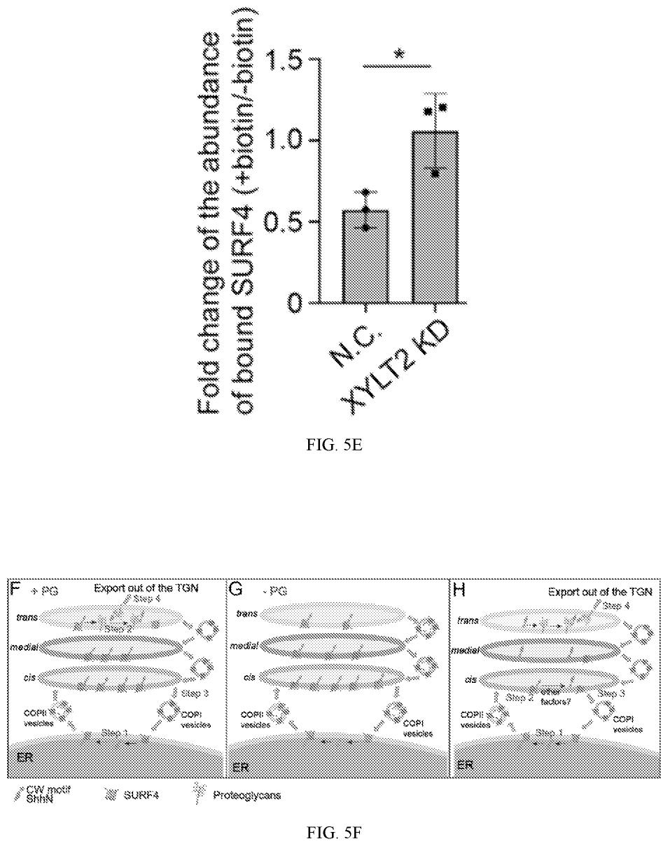

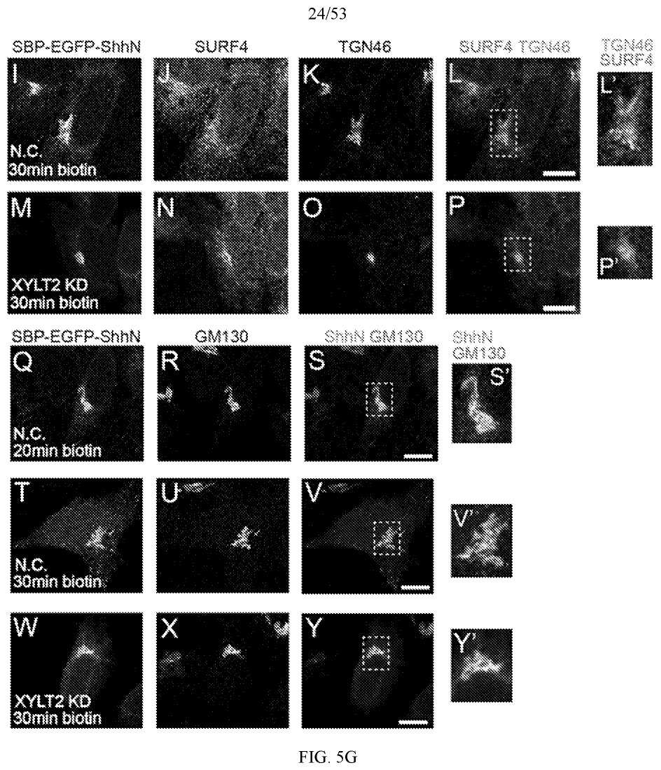

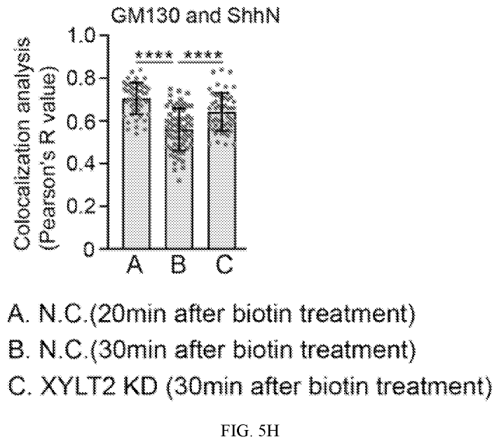

The CW motif of Shh interacts with GAG chains of PGs (37, 38). Mutating this motif causes defects in hedgehog signaling in mice (37, 39). Using GST pull downs, we found that the addition of a GAG, heparin, inhibited the interaction between ShhN and SURF4 in a concentration-dependent manner (FIG. 5A and quantification in FIG. 5B), suggesting heparin competes with SURF4 to bind ShhN. As GAG chains are attached to the PG core proteins in the Golgi, we hypothesized that this competition mediates the dissociation of SURF4 from ShhN at the Golgi. To test this hypothesis, we performed crosslinking co-IP experiments in the absence (ER-localized complex) and presence (Golgi-localized complex) of biotin. Precipitation of SURF4-Myc with the Golgi-localized ShhN RUSH construct was reduced relative to that co-precipitated in the ER-localized condition (FIG. 5C). In contrast, in XYLT2 knockdown cells, the abundance of SURF4 that associated with the Golgi-localized SBP-EGFP-ShhN was equivalent to that co-precipitating with the ER-localized pool (FIG. 5D; quantification in FIG. 5E). These analyses suggest that blocking PG synthesis causes defects in the dissociation of SURF4 from the ShhN at the Golgi.

The SURF4-ShhN complex, after being delivered to the Golgi, will dissociate via a competitive interaction with PGs. SURF4 would then be retrieved back to the ER via COPI vesicles, and ShhN in association with PGs would be exported toward the cell surface (FIG. 5F panel F). This model predicts that defects in the dissociation of SURF4 from ShhN at the Golgi may result in two possible consequences: 1) SURF4 penetrates to the TGN area in XYLT2 knockdown cells after biotin treatment; or 2) accumulation of ShhN at the cis Golgi together with its associated SURF4 (FIG. 5F panel G). To test the first possibility, we analyzed the colocalization between endogenous SURF4 and TGN46 in XYLT2 KD cells 30 min after biotin treatment. However, we did not detect a clear colocalization between SURF4 and TGN46 in control cells or in the knockdown cells (FIG. 5G panels I-P, magnified views in panels L′ and P′). SURF4 is rapidly and constitutively retrieved back to the cis Golgi after the SURF4-ShhN complex is delivered to the Golgi, making TGN-located SURF4 difficult to detect.

To test the second possibility, we analyzed trafficking of SBP-EGFP-ShhN through the cis Golgi in cells treated with biotin for 20 min or 30 min. We quantified the colocalization between SBP-EGFP-ShhN and GM130 in the juxtanuclear area labelled by SBP-EGFP-ShhN. We found that the colocalization between GM130 and the juxtanuclear-located SBP-EGFP-ShhN was significantly reduced in cells treated with biotin for 30 min than in cells treated with biotin for 20 min (FIG. 5G panels Q-V, magnified views in panels S′ and V′, quantifications in FIG. 5H), demonstrating the passage of SBP-EGFP-ShhN out of the cis Golgi during biotin treatment. In XYLY2 KD cells, we found that colocalization between SBP-EGFP-ShhN and GM130 was significantly higher than that in control cells 30 min after biotin treatment (FIG. 5G panels T-Y, magnified views in panels V′ and Y′, quantifications in FIG. 5H). This analysis suggests that blocking PG synthesis causes defects in trafficking of ShhN beyond the cis Golgi.

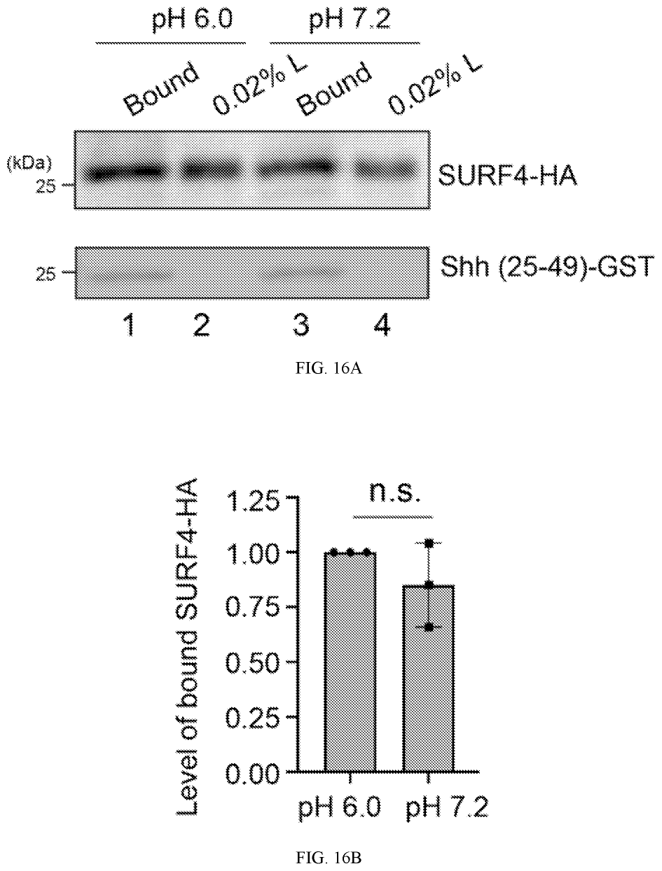

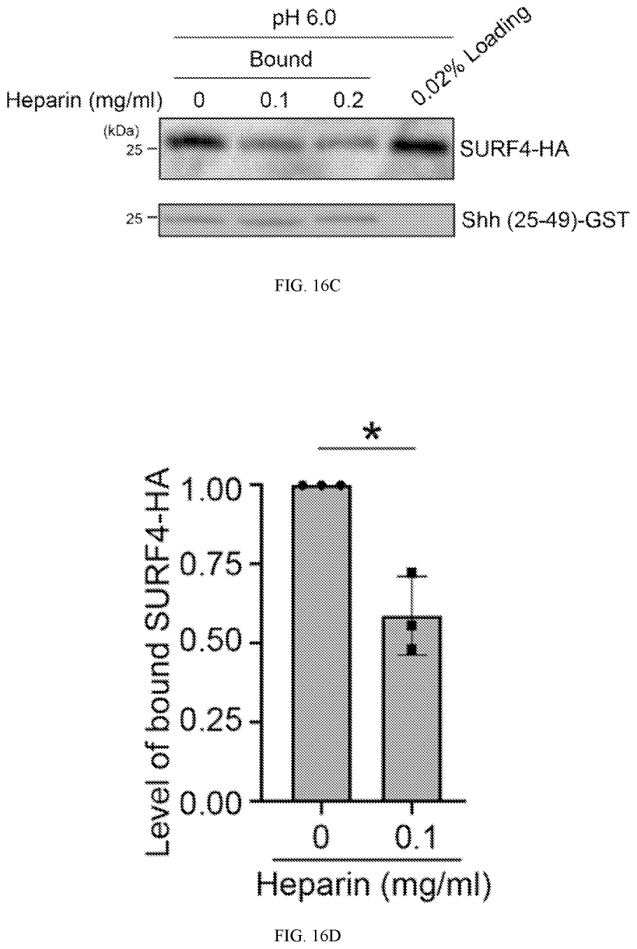

The protein interaction and colocalization analyses provide evidence suggesting that displacement of SURF4 from ShhN by PGs is important for trafficking of ShhN through the Golgi. In addition to this mechanism, many soluble cargo proteins are dissociated from their receptors at low pH (40, 41). The luminal pH of the ER is nearly neutral and the luminal pH of the TGN is around 6.0 (42). We found that lowering the pH from 7.2 to 6.0 did not cause a significant reduction of the abundance of SURF4-HA that bound Shh (25-49)-GST (FIG. 16A, compare lanes 1 and 3, quantification in FIG. 16B), suggesting the slightly acidic pH at the TGN is unlikely to drive release of SURF4 from ShhN. At pH 6.0, heparin still inhibits the ShhN-SURF4 interaction (FIGS. 16C-16D) indicating that PGs can compete SURF4 to bind ShhN in the slightly acidic environment at the TGN. However, we cannot rule out the possibility that other molecules also contribute to release of SURF4 from ShhN at the Golgi, with the released ShhN subsequently engaging with PGs at the TGN for onward traffic (FIG. 5G panel H). The analyses here also indicate that delays in intra-Golgi transport of ShhN in XYLT2 knockdown cells may indirectly interfere with TGN export.

### Example 7—SURF4 and Synthesis of PGS Are Important for ER Export and TGN Export of Full Length SHH Respectively

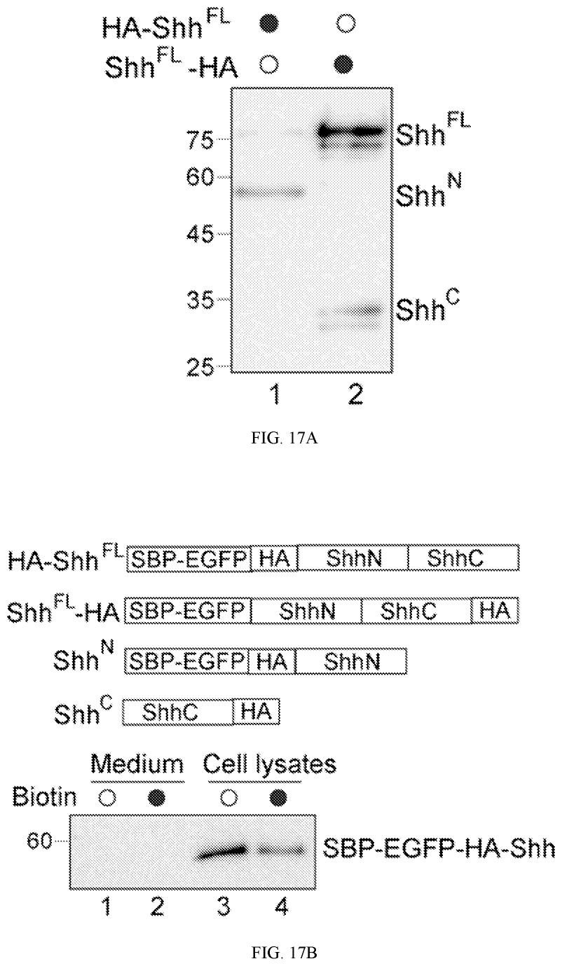

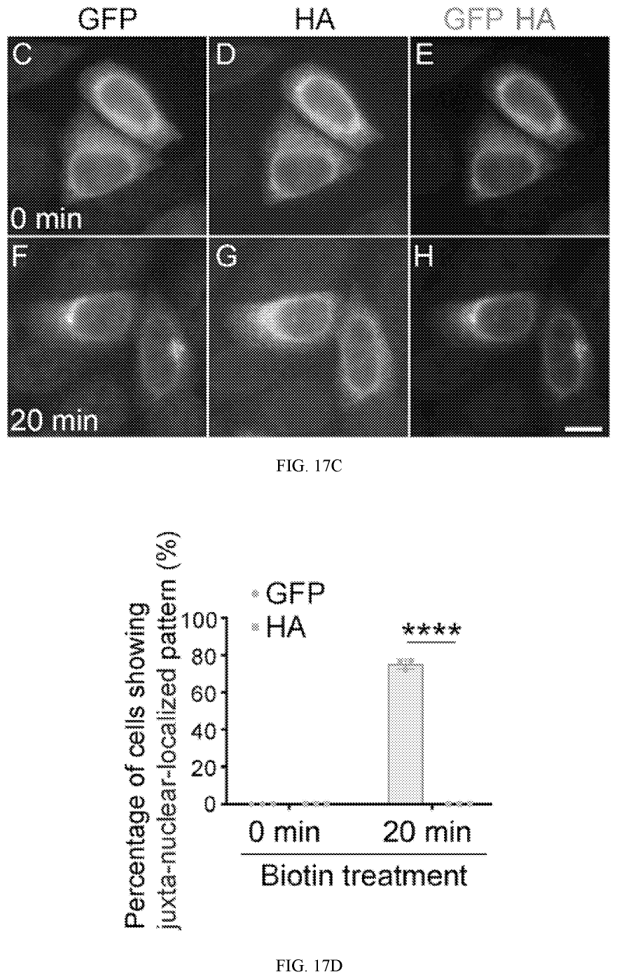

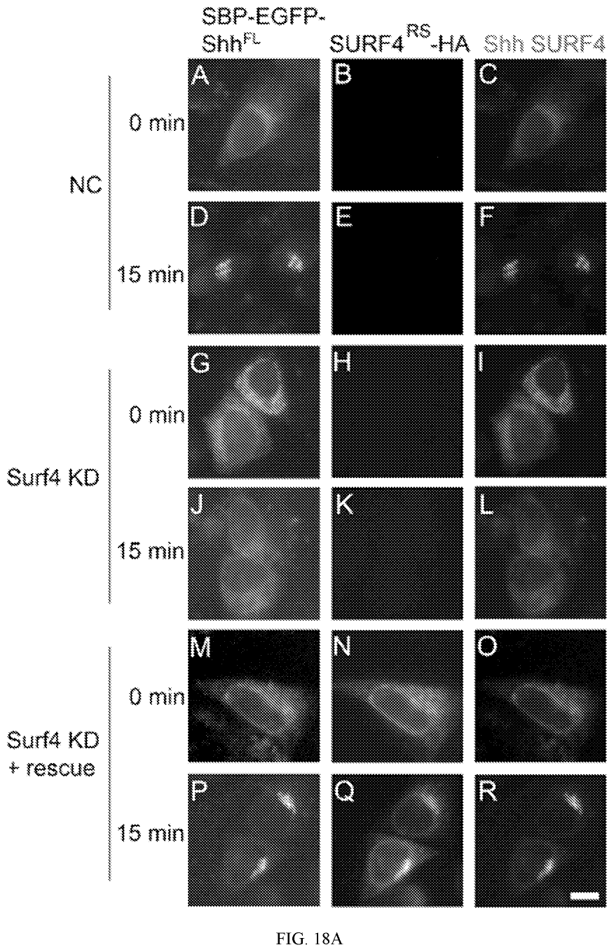

Since the ShhN construct we used is not modified by cholesterol, we wanted to test the effects of SURF4 under more native conditions. We therefore generated a RUSH construct of full length Shh (SBP-EGFP-Shh^{FL}). To test whether the proteins encoded by the RUSH construct can be processed into the N- and C-terminal fragments, HEK293T cells were transfected with the RUSH construct of ShhFL bearing a N-terminal or C-terminal HA tag (SBP-EGFP-HA-Shh^{FL} or SBP-EGFP-ShhFL-HA). Immunoblot analyses showed that two bands can be detected by anti-HA antibody in cell lysates from HEK293T cells expressing SBP-EGFP-HA-Shh^{FL} (HA-Shh^{FL}). Their molecular weights matched those predicted for the N-terminal fragment and full-length precursor of Shh, SBP-EGFP-HA-ShhN (˜54 kDa) and SBP-EGFP-HA-Shh^{FL} (˜80 kDa) (FIG. 17A, lane 1). Two major bands can be detected by anti-HA antibody in cell lysates from HEK293T cells expressing SBP-EGFP-Shh^{FL}-HA (Shh^{FL}-HA). The molecular weights matched the predicted C-terminal fragment and full-length precursor of Shh, ShhC-HA (˜33 kDa) and SBP-EGFP-Shh^{FL}-HA (˜80 kDa) (FIG. 17A, lane 2). These analyses indicate that SBP-EGFP-Shh^{FL} is processed into N-terminal and C-terminal fragments. SBP-EGFP-Shh^{FL} was not detected in the media fraction from cell cultures treated with biotin (FIG. 17B). We suggest that the N-terminal fragment generated by cleavage of SBP-EGFP-HA-Shh^{FL} is modified by cholesterol and this modification prevents the release of the N-terminal fragment from the plasma membrane to the medium. We also detected a reduction in the abundance of the processed RUSH fusion of Shh^{FL} in cell lysates after biotin treatment (compare lanes 3 and 4 in FIG. 17B). We hypothesize that some of the N-terminal fragment of Shh, after delivery to the cell surface, is internalized and routed to lysosomes for degradation. We therefore performed immunofluorescence to visualize the localization of processed Shh fragments in Hela cells expressing SBP-EGFP-Shh^{FL}-HA. The processed N-terminal fragment of Shh was annotated as SBP-EGFP-ShhN, and the processed C-terminal domain of Shh was annotated as ShhC-HA. SBP-EGFP-ShhN and ShhC-HA were located at the ER in the absence of biotin (FIG. 17C panels C-E). 20 min after biotin treatment, ˜80% cells showed Golgi-localized SBP-EGFP-ShhN, whereas ShhC-HA was still in the ER (FIG. 17C panels F-H and quantification in FIG. 17D). These results indicate that the N-terminal fragment of SBP-EGFP-ShhFL can be transported from the ER to the Golgi, while the C-terminal fragment cannot.

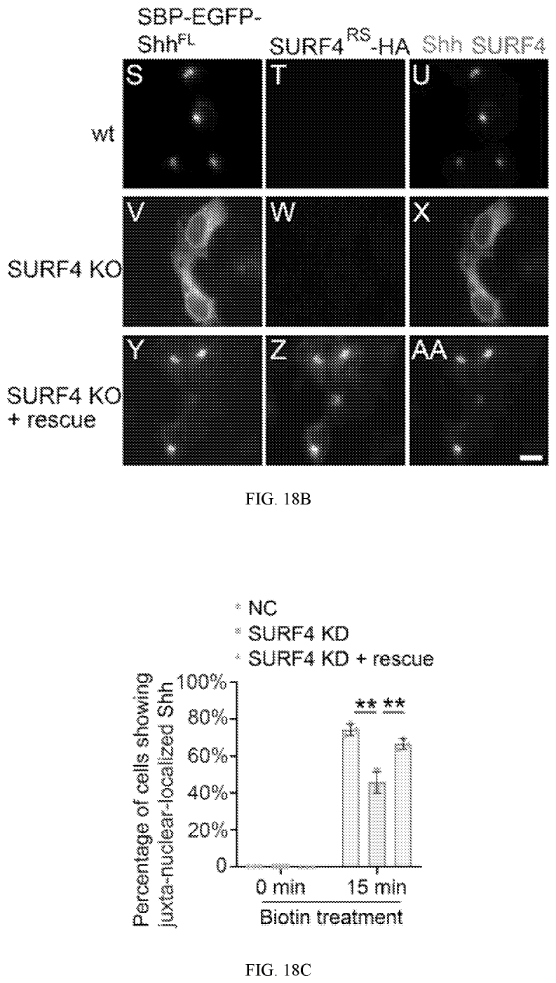

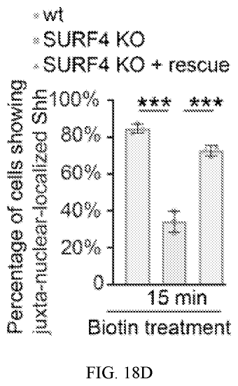

We next used the RUSH constructs of Shh^{FL} to analyze ER export and TGN export. We found that SBP-EGFP-Shh^{FL} can be delivered to the juxtanuclear Golgi area in a biotin-dependent manner (FIG. 18A panels A-F). The kinetics of ER-to-Golgi transport of SBP-EGFP-Shh^{FL} was significantly reduced in SURF4 knockdown cells and this defect was rescued by expressing siRNA-resistant SURF4-HA (FIG. 18A panels G-R and quantification in FIG. 18C). ER-to-Golgi trafficking defects were also observed in SURF4 KO cells (FIG. 18B panels S-X and quantification in FIG. 18D) and this defect was rescued by expressing the siRNA-resistant SURF4-HA (FIG. 18B panels Y-AA and quantification in FIG. 18D).

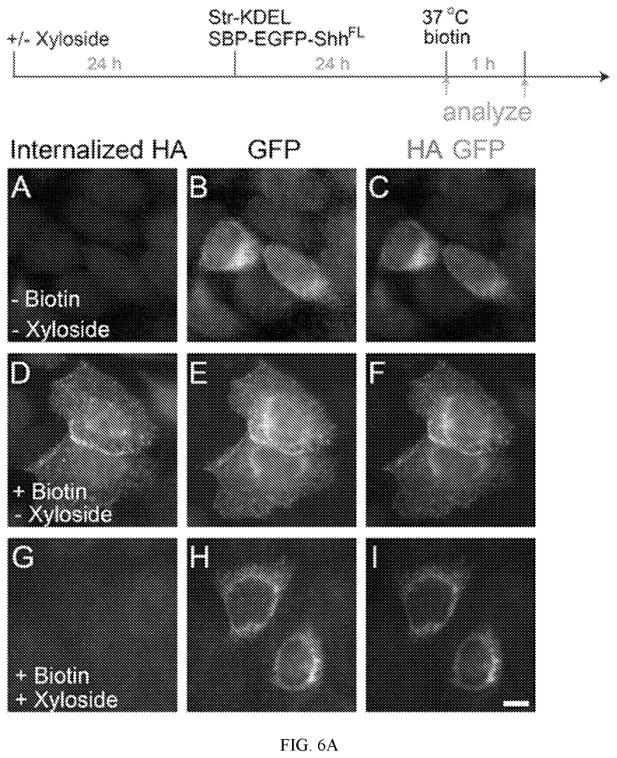

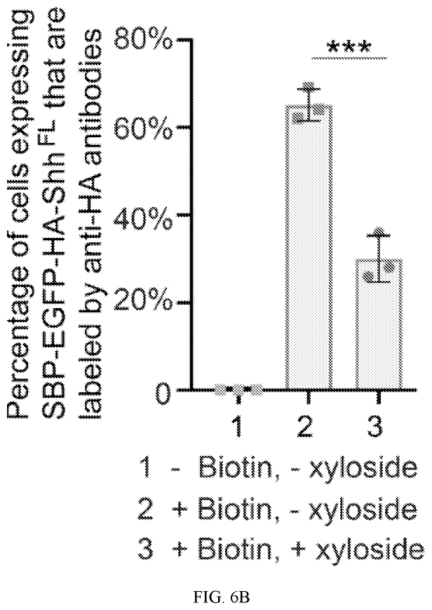

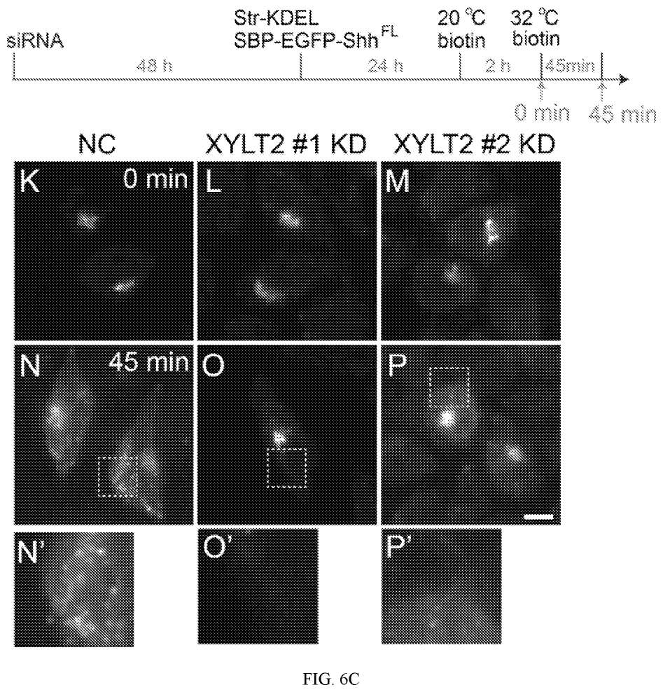

SBP-EGFP-Shh^{FL} is not clearly detectable on the cell surface after biotin treatment presumably because the membrane-anchored protein is rapidly internalized after delivery to the plasma membrane. To test whether synthesis of PG is important for surface delivery of Shh, we utilized the RUSH construct of Shh that contains an HA tag (SBP-EGFP-HA-Shh^{FL}) and performed antibody uptake experiments. Mouse anti-HA antibodies were used to label SBP-EGFP-HA-Shh^{FL} that had been delivered to the cell surface, and rabbit anti-GFP antibodies were used to label the total signal of SBP-EGFP-HA-Shh^{FL}. The Shh-expressing cells were not detected by mouse anti-HA antibodies in the absence of biotin (FIG. 6A panels A-C, FIG. 6B). 1 h after biotin treatment, around 65% of the Shh-expressing cells were detected by anti-HA antibodies in the absence of xyloside (FIG. 6A panels D-F, FIG. 6B). In contrast, the percentage of Shh-expressing cells detected by anti-HA antibodies was significantly lower in the presence of xyloside after biotin treatment (65% vs. 30%, FIG. 6A panels G-I, FIG. 6B). This result indicates that blocking PG synthesis causes defects in surface delivery of SBP-EGFP-HA-Shh^{FL}. SBP-EGFP-HA-Shh^{FL} showed a juxtanuclear-located pattern and ER-like pattern in many of the cells treated with xyloside after biotin treatment (FIG. 6A panel H). The continued localization of SBP-EGFP-HA-Shh^{FL} at the ER after biotin treatment may be caused by incomplete cycloheximide effectiveness, or by inappropriate Golgi-ER retrieval of the SURF4-Shh complex in xyloside-treated cells.

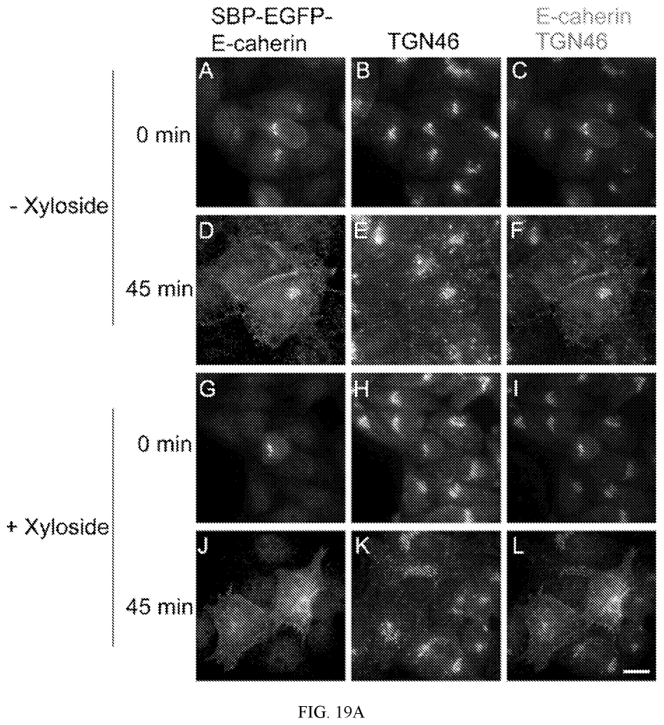

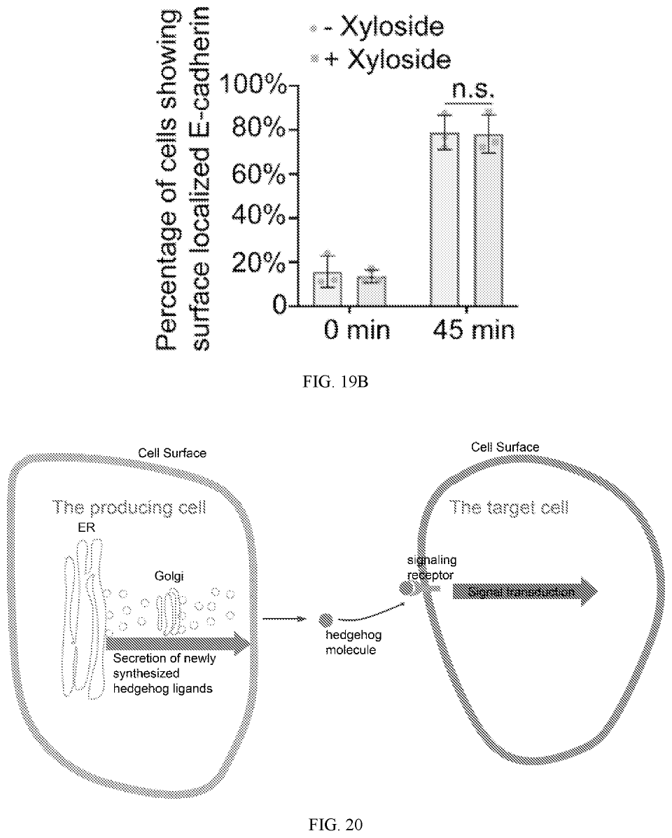

To demonstrate that xyloside treatment does not cause global secretory defects, we analyzed trafficking of a RUSH construct of E-cadherin (SBP-EGFP-E-cadherin). After 20° C. incubation, SBP-EGFP-E-cadherin accumulated at the juxtanuclear Golgi area (FIG. 19A panels A-C and FIG. 19A panels G-I). 45 min after incubation at 32° C., SBP-EGFP-E-cadherin was detectable on the cell surface or cell junction in the majority of cells (FIG. 19A panels D-F, FIG. 19B). Xyloside treatment did not cause defects in surface delivery of SBP-EGFP-E-cadherin after incubation at 32° C. (FIG. 19A panels J-L, and quantification in FIG. 19B), suggesting that blocking PG synthesis does not block TGN-to-cell surface delivery of E-cadherin.

Finally, we performed a temperature shift experiment by incubating cells in the presence of biotin at 20° C. for 2 h and then 32° C. for 45 min. We found that the average number of punctate structures containing SBP-EGFP-Shh in each cell was significantly decreased after XYLT2 knockdown (FIG. 6C panels K-P, and magnified views in panels N′-P′, FIG. 6D). These analyses suggest that synthesis of PGs is important for the TGN export of the RUSH construct of Shh^{FL}.

### Example 8—Shh Trafficking

Regulating the release of newly synthesized signaling molecules by modulating their secretion can influence downstream signaling pathways in the target cells. Although fundamentally important, the underlying molecular mechanisms that mediate the biosynthetic trafficking of signaling molecules remain largely unclear. We analyzed the trafficking of a secreted signaling molecule, Shh. The sorting and secretion of newly synthesized Shh is achieved in several steps (FIG. 5F): 1) a cargo receptor, SURF4, interacts with the CW motif of Shh to package ShhN into COPII vesicles at the ER (step 1); 2) after being delivered to the Golgi, the SURF4-ShhN complex is dissociated (step 2); 3) the released SURF4 is retrieved to the ER by COPI vesicles (step 3); 4) the released ShhN associates with PGs and is exported out of the TGN (step 4). We found that PGs compete with SURF4 to bind ShhN, and defects in PG synthesis enhance the association between SURF4 and ShhN at the Golgi. These analyses suggest that PGs are important factors regulating the displacement of SURF4 from ShhN at the Golgi (FIG. 5F panel F). In addition to PGs, other factors, such as other charged molecules may also contribute to the dissociation of SURF4 from ShhN at the Golgi (FIG. 5F panel H).

Selective retention of proteins in the ER and capturing of cargo proteins in COPII vesicles have been shown to regulate the specificity of ER export (18). In addition to selective capture, cargo proteins can also exit the ER through bulk flow (18). In this mechanism, inclusion of cargo proteins into COPII vesicles occurs by default and is not dependent on receptors or export signals. Utilizing the RUSH assay, we found that EGFP without the CW motif is not transported from the ER to the Golgi after 30 min of biotin treatment (FIG. 7D panels AC-AE). In contrast, the CW motif of Shh is sufficient for efficient export of EGFP out of the ER (FIGS. 1A-1B). These analyses indicate that EGFP is not subject to significant forward transport by bulk flow.

After being delivered to the target compartment, cargo proteins need to be dissociated from their receptors, which are recycled back to the donor compartment to perform another round of cargo sorting. One mechanism that regulates this dissociation is a pH-sensitive ligand uncoupling mechanism (40, 41). This mechanism regulates the dissociation of soluble acid hydrolase precursor and their receptor Mannose 6-phosphate receptor (M6PR) in endosomes so that M6PR can be retrieved to the TGN to mediate the next cycle of hydrolase trafficking (41). In this study, we revealed a direct electrostatic interaction between the CW motif and negatively charged residues located in the predicted first luminal loop of SURF4. We provide evidence suggesting that PGs compete with SURF4 to interact with the CW motif in Shh at the Golgi, providing a new way for dissociating cargo proteins from cargo receptors.

In addition to trafficking of Shh, SURF4 also mediates the export of other soluble proteins, including lipoproteins, PCSK9, and extracellular dentins from the ER. It also participates in ERES organization and interacts with amino-terminal hydrophobic-proline-hydrophobic motifs of soluble cargo proteins (19, 20, 22). The N-terminal tripeptide motif interacts with a domain on SURF4 that is distinct from the CW-motif binding site on SURF4. Another possibility is that the N-terminal tripeptide motif interact indirectly with SURF4 through an unknown cellular factor. As SURF4 contains a C-terminal retrieval signal (21), the C-terminal HA-tagged SURF4 may not has the maximal capacity as SURF4 without the HA tag. We found that SURF4-HA traffics together with ShhN from the ER to the Golgi and rescued the defects of ER-to-Golgi trafficking of ShhN in the RUSH assay. These analyses suggest that SURF4-HA is functional to promote ER-to-Golgi transport of ShhN, although it may not possess the maximum capacity as an untagged version of SURF4. Knockout of Shh causes embryonic lethality and induces defects in patterning of embryonic tissues, including the brain and eye, the spinal cord, the axial skeleton structures and the limbs (43). Knockout of SURF4 also results in early embryonic lethality in mice with loss of all knockout embryos between embryonic days 3.5 and 9.5 (44). Our results suggest that Shh is a key SURF4 client, and that knockout of SURF4 causes defects in the secretion of Shh, which contributes to defects in early embryonic development.

Proteoglycans are composed of core proteins linked to the GAG family of sugars, which includes heparan sulfate, dermatan sulfate, keratin sulfate and chondroitin sulfate (45). All have been shown to interact with a variety of signaling molecules (45). These interactions regulate the free diffusion of signaling molecules and allow the proteoglycans to function as signal coreceptors to regulate signal transduction (46). In Drosophila, a cell surface located heparan sulfate proteoglycan (HSPG), glypican, regulates the association of hedgehog with lipoproteins to facilitate the release of hedgehog in lipoprotein particles and thereby regulates the spread of hedgehog through a tissue (47). Heparan sulfate chains have been shown to regulate metalloprotease-mediated Shh release from producing cells (48) and hedgehog signaling in target cells (47). The CW motif of Shh is important for the interaction with heparan sulfate chains (37, 38). Mutations in this motif (R34A/K38A) in Shh reduce the affinity between Shh and proteoglycan in the cerebellum and decrease Shh-induced proliferation of granule cells in mice in situ (37). Interestingly, R34A/K38A mutations in Shh cause defects in proliferation of neural precursor cells, but not in tissue patterning (39). In this study, we revealed that the CW motif can be sequentially recognized by SURF4 and proteoglycans to mediate its surface delivery. In addition to ShhN, other CW motif-containing secretory proteins include bone morphogenetic protein (BMP) 8A, BMP8B, and extracellular sulfatase (Sulf)-1. We hypothesize that the SURF4-proteoglycan relay mechanism may provide a general regulation for the ER-Golgi transport of CW motif-containing proteins.

PGs regulate TGN export of ShhN by two possible non-mutually exclusive mechanisms: 1) deficiencies in PG synthesis induce delays in intra-Golgi transport of ShhN, which indirectly interfere with subsequent TGN export; 2) PG functions as a cargo receptor that regulates TGN sorting of Shh. The integral membrane proteoglycan Syndecan-1 (SDC1) acts as a cargo receptor that regulates TGN sorting of lipoprotein lipase (LPL) (36). SDC1 and LPL are co-secreted in secretory vesicles enriched in sphingomyelin (SM) (36). Physical features of the SDC1 transmembrane domain drives association with the SM-rich membrane of the TGN, and that this association concentrates SDC1 and its associated LPL, thereby targeting SDC1 and bound LPL into the sphingomyelin secretion pathway (36).

The α-amino group of the cysteine residue at the N-terminus of Shh is modified by palmitoylation catalyzed by Hedgehog acyltransferase (15, 49). The palmitoylation modification requires an N-terminal cysteine with a free amino group (15). In the RUSH construct of Shh^{FL} or ShhN, the α-amino group of the cysteine residue at the N-terminus of Shh forms a peptide bond with the SBP-GFP tag, suggesting the RUSH constructs of ShhN or Shh^{FL} utilized in our study are not modified by palmitoylation. The RUSH construct of ShhN can be efficiently secreted, indicating that the palmitoylation modification is not required for the secretion of Shh.

In this study, we used mammalian cells exogenously expressing a specific cargo protein as a system to investigate the molecular mechanisms of cargo sorting. This system is convenient to perform biochemical and cell biological approaches to reveal novel mechanistic insights. Ligand production by tumor cells or the surrounding stroma has been demonstrated to activate the Hh signaling pathway to promote tumorigenesis (50-52). The protein interactions identified in our study that mediate the sorting and secretion of Shh provide novel therapeutic targets to downregulate Hh signaling for cancer treatment by inhibiting the secretion of Shh.

SEQUENCES
SEQ ID NO: 1:
SURF4 sgRNA
(AGTCGCGCTGCTCGCTCCAC)
SEQ ID NO: 2:
siRNA against SURF4
(GCAGGAACTTCGTGCAGTA)
SEQ ID NO: 3:
siRNA against SURF4
(GCATCCGTATGTGGTTCCA)
SEQ ID NO: 4:
siRNA against XYLT2
(CTGGTAGTGTGGAGCTTCA)
SEQ ID NO: 5:
siRNA against XYLT2
(GCGTGCACCTGTATTTCTA)
SEQ ID NO: 6:
Cardin-Weintraub peptide
(KRRHPKKC)
SEQ ID NO: 7:
Cardin-Weintraub peptide
(RRRHPKK)
SEQ ID NO: 8:
Cardin-
Weintraub peptide
(KRRHPKK)
SEQ ID NO: 9:
Cardin-Weintraub peptide^{MT} peptide
(AAAHPAAC)
SEQ ID NO: 10:
SURF4 peptide:
(SEQRDYIDTTWNC)
SEQ ID NO: 11:
RRFR peptide
(VRRFRYPERPC)
SEQ ID NO: 12:
SURF4 peptide
(SEQRDYIDTTWN)
SEQ ID NO: 13:
Nucleotide sequence encoding Cardin-
Weintraub peptide of SEQ ID NO: 8
(AAGAGGAGGCACCCCAAAAAG)
SEQ ID NO: 14:
Nucleotide sequence encoding SURF4
peptide of SEQ ID NO: 10
(AGCGAGCAGCGCGACTACATCGACACCACCTGGAACTGC)
SEQ ID NO: 15:
Human SURF4 amino acid sequence
(MGQNDLMGTAEDFADQFLRVTKQYLPHVARLCLISTFLEDGIRM
WFQWSEQRDYIDTTWNCGYLLASSFVFLNLLGQLTGCVLVLSRNF
VQYACFGLFGIIALQTIAYSILWDLKFLMRNLALGGGLLLLLAES
RSEGKSMFAGVPTMRESSPKQYMQLGGRVLLVLMFMTLLHFDASF
FSIVQNIVGTALMILVAIGFKTKLAALTLVVWLFAINVYFNAFWT
IPVYKPMHDFLKYDFFQTMSVIGGLLLVVALGPGGVSMDEKKKEW)
SEQ ID NO: 16:
Human SURF4 cDNA nucleotide sequence
(ATGGGCCAGAACGACCTGATGGGCACGGCCGAGGACTTCGCCGA
CCAGTTCCTCCGTGTCACAAAGCAGTACCTGCCCCACGTGGCGCG
CCTCTGTCTGATCAGCACCTTCCTGGAGGACGGCATCCGTATGTG
GTTCCAGTGGAGCGAGCAGCGCGACTACATCGACACCACCTGGAA
CTGCGGCTACCTGCTGGCCTCGTCCTTCGTCTTCCTCAACTTGCT
GGGACAGCTGACTGGCTGCGTCCTGGTGTTGAGCAGGAACTTCGT
GCAGTACGCCTGCTTCGGGCTCTTTGGAATCATAGCTCTGCAGAC
GATTGCCTACAGCATTTTATGGGACTTGAAGTTTTTGATGAGGAA
CCTGGCCCTGGGAGGAGGCCTGTTGCTGCTCCTAGCAGAATCCCG
TTCTGAAGGGAAGAGCATGTTTGCGGGCGTCCCCACCATGCGTGA
GAGCTCCCCCAAACAGTACATGCAGCTCGGAGGCAGGGTCTTGCT
GGTTCTGATGTTCATGACCCTCCTTCACTTTGACGCCAGCTTCTT
TTCTATTGTCCAGAACATCGTGGGCACAGCTCTGATGATTTTAGT
GGCCATTGGTTTTAAAACCAAGCTGGCTGCTTTGACTCTTGTTGT
GTGGCTCTTTGCCATCAACGTATATTTCAACGCCTTCTGGACCAT
TCCAGTCTACAAGCCCATGCATGACTTCCTGAAATACGACTTCTT
CCAGACCATGTCGGTGATTGGGGGCTTGCTCCTGGTGGTGGCCCT
GGGCCCTGGGGGTGTCTCCATGGATGAGAAGAAGAAGGAGTGGTA
A)
SEQ ID NO: 17:
Human Sonic Hedgehog amino acid sequence
(MLLLARCLLLVLVSSLLVCSGLACGPGRGFGKRRHPKKLTPLAY
KQFIPNVAEKTLGASGRYEGKISRNSERFKELTPNYNPDIIFKDE
ENTGADRLMTQRCKDKLNALAISVMNQWPGVKLRVTEGWDEDGHH
SEESLHYEGRAVDITTSDRDRSKYGMLARLAVEAGFDWVYYESKA
HIHCSVKAENSVAAKSGGCFPGSATVHLEQGGTKLVKDLSPGDRV
LAADDQGRLLYSDFLTFLDRDDGAKKVFYVIETREPRERLLLTAA
HLLEVAPHNDSATGEPEASSGSGPPSGGALGPRALFASRVRPGQR
VYVVAERDGDRRLLPAAVHSVTLSEEAAGAYAPLTAQGTILINRV
LASCYAVIEEHSWAHRAFAPFRLAHALLAALAPARTDRGGDSGGG
DRGGGGGRVALTAPGAADAPGAGATAGIHWYSQLLYQIGTWLLDS
EALHPLGMAVKSS)
SEQ ID NO: 18:
Human Sonic Hedgehog cDNA nucleotide sequence
(ATGCTGCTGCTGGCGAGATGTCTGCTGCTAGTCCTCGTCTCCTC
GCTGCTGGTATGCTCGGGACTGGCGTGCGGACCGGGCAGGGGGTT
CGGGAAGAGGAGGCACCCCA(AAAAGCTGACCCCTTTAGCCTACA
AGCAGTTTATCCCCAATGTGGCCGAGAAGACCCTAGGCGCCAGCG
GAAGGTATGAAGGGAAGATCTCCAGAAACTCCGAGCGATTTAAGG
AACTCACCCCCAATTACAACCCCGACATCATATTTAAGGATGAAG
AAAACACCGGAGCGGACAGGCTGATGACTCAGAGGTGTAAGGACA
AGTTGAACGCTTTGGCCATCTCGGTGATGAACCAGTGGCCAGGAG
TGAAACTGCGGGTGACCGAGGGCTGGGACGAAGATGGCCACCACT
CAGAGGAGTCTCTGCACTACGAGGGCCGCGCAGTGGACATCACCA
CGTCTGACCGCGACCGCAGCAAGTACGGCATGCTGGCCCGCCTGG
CGGTGGAGGCCGGCTTCGACTGGGTGTACTACGAGTCCAAGGCAC
ATATCCACTGCTCGGTGAAAGCAGAGAACTCGGTGGCGGCCAAAT
CGGGAGGCTGCTTCCCGGGCTCGGCCACGGTGCACCTGGAGCAGG
GCGGCACCAAGCTGGTGAAGGACCTGAGCCCCGGGGACCGCGTGC
TGGCGGCGGACGACCAGGGCCGGCTGCTCTACAGCGACTTCCTCA
CTTTCCTGGACCGCGACGACGGCGCCAAGAAGGTCTTCTACGTGA
TCGAGACGCGGGAGCCGCGCGAGCGCCTGCTGCTCACCGCCGCGC
ACCTGCTCTTTGTGGCGCCGCACAACGACTCGGCCACCGGGGAGC
CCGAGGCGTCCTCGGGCTCGGGGCCGCCTTCCGGGGGCGCACTGG
GGCCTCGGGCGCTGTTCGCCAGCCGCGTGCGCCCGGGCCAGCGCG
TGTACGTGGTGGCCGAGCGTGACGGGGACCGCCGGCTCCTGCCCG
CCGCTGTGCACAGCGTGACCCTAAGCGAGGAGGCCGCGGGCGCCT
ACGCGCCGCTCACGGCCCAGGGCACCATTCTCATCAACCGGGTGC
TGGCCTCGTGCTACGCGGTCATCGAGGAGCACAGCTGGGCGCACC
GGGCCTTCGCGCCCTTCCGCCTGGCGCACGCGCTCCTGGCTGCAC
TGGCGCCCGCGCGCACGGACCGCGGCGGGGACAGCGGCGGCGGGG
ACCGCGGGGGCGGCGGCGGCAGAGTAGCCCTAACCGCTCCAGGTG
CTGCCGACGCTCCGGGTGCGGGGGCCACCGCGGGCATCCACTGGT
ACTCGCAGCTGCTCTACCAAATAGGCACCTGGCTCCTGGACAGCG
AGGCCCTGCACCCGCTGGGCATGGCGGTCAAGTCCAGCTGA)

It should be understood that the examples and embodiments described herein are for illustrative purposes only and that various modifications or changes in light thereof will) be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### EXEMPLIFIED EMBODIMENTS

The invention may be better understood by reference to certain illustrative examples, including but not limited to the following:
Embodiment 1. A polypeptide molecule selected from the group comprising of:
i) a polypeptide molecule comprising SEQ ID NOs: 6-8;
ii) a polypeptide molecule comprising SEQ ID NO: 10; or
iii) a polypeptide molecule that has at least 90% sequence identity with the polypeptide of (i) or (ii).

Embodiment 2. An isolated polynucleotide encoding the polypeptide of embodiment 1.

Embodiment 3. The polypeptide of embodiment 1, wherein polypeptide blocks the interaction between Surfeit locus protein 4 (SURF4) and Sonic hedgehog (Shh).

Embodiment 4. The polypeptide of embodiment 1, wherein polypeptide binds to Shh.

Embodiment 5. A composition comprising the polypeptide of embodiment 1.

Embodiment 6. The composition of embodiment 5, further comprising a pharmaceutically acceptable carrier and/or excipient.

Embodiment 7. A method for inhibiting the Hh signaling pathway, the method comprising administering a composition comprising a large molecule that blocks the interaction between SURF4 and Shh or the polypeptide molecule of embodiment 1 to a subject.

Embodiment 8. The method of embodiment 7, wherein the polypeptide molecule blocks the interaction between SURF4 and Shh.

Embodiment 9. The method of embodiment 7, wherein the polypeptide molecule binds to Shh.

Embodiment 10. The method of embodiment 7, wherein the large molecule is a glycosaminoglycan.

Embodiment 11. The method of embodiment 10, wherein the glycosaminoglycan is heparin, heparin sulfate, heparin sulfate proteoglycans (HSPG), or chondroitin sulfate proteoglycans (CSPGs).

Embodiment 12. The method of embodiment 7, wherein the subject has a ligand-dependent cancer.

Embodiment 13. The method of embodiment 7, wherein the subject is a human.

Embodiment 14. The method of embodiment 7, wherein the composition further comprises a pharmaceutically acceptable carrier and/or excipient.

Embodiment 15. A method for inhibiting the Hh signaling pathway, the method comprising mutating in a subject:
i) amino acid residue E50, D53, D56, or any combination thereof of human SURF4;
ii) a nucleotide encoding amino acid residues E50, D53, D56, or any combination thereof of human SURF4;
iii) amino acid residue 32, 33, 34, 35, 36, 37, 38, of human Shh or any combination thereof; or
iv) a nucleotide encoding amino acid residue 32, 33, 34, 35, 36, 37, 38, or any combination thereof of human Shh.

Embodiment 16. The method of embodiment 15, wherein the mutations of i)-iv) inhibit the interaction of SURF4 and Shh in a subject.

Embodiment 17. The method of embodiment 15, wherein the subject has a ligand-dependent cancer.

Embodiment 18. The method of embodiment 15, wherein the subject is a human.

### REFERENCES

1. P. W. Ingham, Y. Nakano, C. Seger, Mechanisms and functions of Hedgehog signalling across the metazoa. Nat Rev Genet 12, 393-406 (2011).
2. R. Petrova, A. L. Joyner, Roles for Hedgehog signaling in adult organ homeostasis and repair. Development 141, 3445-3457 (2014).
3 J. L. Mullor, P. Sanchez, A. Ruiz i Altaba, Pathways and consequences: Hedgehog signaling in human disease. Trends Cell Biol 12, 562-569 (2002).
4. P. W. Ingham, M. Placzek, Orchestrating ontogenesis: variations on a theme by sonic hedgehog. Nat Rev Genet 7, 841-850 (2006).
5. B. St-Jacques, M. Hammerschmidt, A. P. McMahon, Indian hedgehog signaling regulates proliferation and differentiation of chondrocytes and is essential for bone formation. Genes Dev 13, 2072-2086 (1999).
6. M. J. Bitgood, L. Shen, A. P. McMahon, Sertoli cell signaling by Desert hedgehog regulates the male germline. Curr Biol 6, 298-304 (1996).
7. H. H. Yao, W. Whoriskey, B. Capel, Desert Hedgehog/Patched 1 signaling specifies fetal Leydig cell fate in testis organogenesis. Genes Dev 16, 1433-1440 (2002).
8. M. Wijgerde, M. Ooms, J. W. Hoogerbrugge, J. A. Grootegoed, Hedgehog signaling in mouse ovary: Indian hedgehog and desert hedgehog from granulosa cells induce target gene expression in developing theca cells. Endocrinology 146, 3558-3566 (2005).
9 E. Dessaud, A. P. McMahon, J. Briscoe, Pattern formation in the vertebrate neural tube: a sonic hedgehog morphogen-regulated transcriptional network. Development 135, 2489-2503 (2008).
10. P. T. Yam, F. Charron, Signaling mechanisms of non-conventional axon guidance cues: the Shh, BMP and Wnt morphogens. Curr Opin Neurobiol 23, 965-973 (2013).
11. A. P. McMahon, P. W. Ingham, C. J. Tabin, Developmental roles and clinical significance of hedgehog signaling. Curr Top Dev Biol 53, 1-114 (2003).
12. X. Chen et al., Processing and turnover of the Hedgehog protein in the endoplasmic reticulum. J Cell Biol 192, 825-838 (2011).
13. I. A. Rose, J. V. Warms, An enzyme with ubiquitin carboxy-terminal esterase activity from reticulocytes. Biochemistry 22, 4234-4237 (1983).
14. J. A. Porter, K. E. Young, P. A. Beachy, Cholesterol modification of hedgehog signaling proteins in animal development. Science 274, 255-259 (1996).
15. J. A. Buglino, M. D. Resh, Hhat is a palmitoylacyltransferase with specificity for N-palmitoylation of Sonic Hedgehog. J Biol Chem 283, 22076-22088 (2008).
16. J. Dancourt, C. Barlowe, Protein sorting receptors in the early secretory pathway. Annu Rev Biochem 79, 777-802 (2010).
17. A. Schweizer, J. A. Fransen, T. Bachi, L. Ginsel, H. P. Hauri, Identification, by a monoclonal antibody, of a 53-kD protein associated with a tubulo-vesicular compartment at the cis-side of the Golgi apparatus. J Cell Biol 107, 1643-1653 (1988).
18. C. Barlowe, A. Helenius, Cargo Capture and Bulk Flow in the Early Secretory Pathway. Annu Rev Cell Dev Biol 32, 197-222 (2016).
19. K. Saegusa, M. Sato, N. Morooka, T. Hara, K. Sato, SFT-4/Surf4 control ER export of soluble cargo proteins and participate in ER exit site organization. J Cell Biol 217, 2073-2085 (2018).
20. B. T. Emmer et al., The cargo receptor SURF4 promotes the efficient cellular secretion of PCSK9. Elife 7 (2018).
21. X. Wang et al., Receptor-Mediated ER Export of Lipoproteins Controls Lipid Homeostasis in Mice and Humans. Cell Metab 10.1016/j.cmet.2020.10.020 (2020).
22. Y. Yin et al., Surf4 (Erv29p) binds amino-terminal tripeptide motifs of soluble cargo proteins with different affinities, enabling prioritization of their exit from the endoplasmic reticulum. PLoS Biol 16, e2005140 (2018).
23. G. Boncompain et al., Synchronization of secretory protein traffic in populations of cells. Nat Methods 9, 493-498 (2012).
24. A. D. Cardin, H. J. Weintraub, Molecular modeling of protein-glycosaminoglycan interactions. Arteriosclerosis 9, 21-32 (1989).
25. M. Aridor, S. I. Bannykh, T. Rowe, W. E. Balch, Sequential coupling between COPII and COPI vesicle coats in endoplasmic reticulum to Golgi transport. J Cell Biol 131, 875-893 (1995).
26. L. Niu et al., Atlastin-mediated membrane tethering is critical for cargo mobility and exit from the endoplasmic reticulum. Proc Natl Acad Sci U S A 116, 14029-14038 (2019).
27. X. Tang et al., Molecular mechanisms that regulate export of the planar cell-polarity protein Frizzled-6 out of the endoplasmic reticulum. J Biol Chem 10.1074/jbc.RA120.012835 (2020).
28. Y. Huang et al., An in vitro vesicle formation assay reveals cargo clients and factors that mediate vesicular trafficking. Proc Natl Acad Sci U S A 118 (2021).
29. Y. Guo, A. D. Linstedt, COPII-Golgi protein interactions regulate COPII coat assembly and Golgi size. J Cell Biol 174, 53-63 (2006).
30. F. Yang, T. Li, Z. Peng, Y. Liu, Y. Guo, The amphipathic helices of Arfrp1 and Arl14 are sufficient to determine subcellular localizations. J Biol Chem 10.1074/jbc.RA120.014999 (2020).
31. S. Otte, C. Barlowe, Sorting signals can direct receptor-mediated export of soluble proteins into COPII vesicles. Nat Cell Biol 6, 1189-1194 (2004).
32. W. J. Belden, C. Barlowe, Role of Erv29p in collecting soluble secretory proteins into ER-derived transport vesicles. Science 294, 1528-1531 (2001).
33. M. Varadi et al., AlphaFold Protein Structure Database: massively expanding the structural coverage of protein-sequence space with high-accuracy models. Nucleic Acids Res 10.1093/nar/gkab 1061 (2021).
34. J. Jumper et al., Highly accurate protein structure prediction with AlphaFold. Nature 596, 583-589 (2021).
35. H. Hughes et al., Organisation of human ER-exit sites: requirements for the localisation of Sec16 to transitional ER. J Cell Sci 122, 2924-2934 (2009).
36. E. L. Sundberg, Y. Deng, C. G. Burd, Syndecan-1 Mediates Sorting of Soluble Lipoprotein Lipase with Sphingomyelin-Rich Membrane in the Golgi Apparatus. Dev Cell 51, 387-398 e384 (2019).
37. J. B. Rubin, Y. Choi, R. A. Segal, Cerebellar proteoglycans regulate sonic hedgehog responses during development. Development 129, 2223-2232 (2002).
38. P. Farshi et al., Dual roles of the Cardin-Weintraub motif in multimeric Sonic hedgehog. J Biol Chem 286, 23608-23619 (2011).
39. J. A. Chan et al., Proteoglycan interactions with Sonic Hedgehog specify mitogenic responses. Nat Neurosci 12, 409-417 (2009).
40. C. G. Davis et al., Acid-dependent ligand dissociation and recycling of LDL receptor mediated by growth factor homology region. Nature 326, 760-765 (1987).
41. P. Ghosh, N. M. Dahms, S. Kornfeld, Mannose 6-phosphate receptors: new twists in the tale. Nat Rev Mol Cell Biol 4, 202-212 (2003).
42. P. Paroutis, N. Touret, S. Grinstein, The pH of the secretory pathway: measurement, determinants, and regulation. Physiology (Bethesda) 19, 207-215 (2004).
43. C. Chiang et al., Cyclopia and defective axial patterning in mice lacking Sonic hedgehog gene function. Nature 383, 407-413 (1996).
44. B. T. Emmer et al., Murine Surf4 is essential for early embryonic development. PLoS One 15, e0227450 (2020).
45. T. Annaval et al., Heparan Sulfate Proteoglycans Biosynthesis and Post Synthesis Mechanisms Combine Few Enzymes and Few Core Proteins to Generate Extensive Structural and Functional Diversity. Molecules 25 (2020).
46. P. Lu, V. M. Weaver, Z. Werb, The extracellular matrix: a dynamic niche in cancer progression. J Cell Biol 196, 395-406 (2012).
47. J. Briscoe, P. P. Therond, The mechanisms of Hedgehog signalling and its roles in development and disease. Nat Rev Mol Cell Biol 14, 416-429 (2013).
48. T. Dierker, R. Dreier, A. Petersen, C. Bordych, K. Grobe, Heparan sulfate-modulated, metalloprotease-mediated sonic hedgehog release from producing cells. J Biol Chem 284, 8013-8022 (2009).
49. R. B. Pepinsky et al., Identification of a palmitic acid-modified form of human Sonic hedgehog. J Biol Chem 273, 14037-14045 (1998).
50. D. M. Berman et al., Widespread requirement for Hedgehog ligand stimulation in growth of digestive tract tumours. Nature 425, 846-851 (2003).
51. R. L. Yauch et al., A paracrine requirement for hedgehog signalling in cancer. Nature 455, 406-410 (2008).
52. M. Niyaz, M. S. Khan, S. Mudassar, Hedgehog Signaling: An Achilles' Heel in Cancer. Transl Oncol 12, 1334-1344 (2019).
53. R. Natarajan, A. D. Linstedt, A cycling cis-Golgi protein mediates endosome-to-Golgi traffic. Mol Biol Cell 15, 4798-4806 (2004).
54. Y. Guo, G. Zanetti, R. Schekman, A novel GTP-binding protein-adaptor protein complex responsible for export of Vangl2 from the trans Golgi network. Elife 2, e00160 (2013).
55. X. Tang, F. Yang, Y. Guo, Cell-free Reconstitution of the Packaging of Cargo Proteins into Vesicles at the trans Golgi Network. Bio Protoc 10, e3537 (2020).
56. G. Boncompain et al., Synchronization of secretory protein traffic in populations of cells. Nat Methods 9, 493-498 (2012).
57. F. Yang, T. Li, Z. Peng, Y. Liu, Y. Guo, The amphipathic helices of Arfrp1 and Arl14 are sufficient to determine subcellular localizations. J Biol Chem 295, 16643-16654 (2020).
58. Y. Guo, G. Zanetti, R. Schekman, A novel GTP-binding protein-adaptor protein complex responsible for export of Vangl2 from the trans Golgi network. Elife 2, e00160 (2013).
59. X. Tang et al., Molecular mechanisms that regulate export of the planar cell-polarity protein Frizzled-6 out of the endoplasmic reticulum. J Biol Chem 10.1074/jbc.RA120.012835 (2020).
60. X. Tang, F. Yang, Y. Guo, Cell-free Reconstitution of the Packaging of Cargo Proteins into Vesicles at the trans Golgi Network. Bio Protoc 10, e3537 (2020).

1. A polypeptide molecule selected from the group consisting of:
i) a polypeptide molecule comprising SEQ ID NOs: 6-8;
ii) polypeptide molecule comprising SEQ ID NO: 10; and
iii) polypeptide molecules that have at least 90% sequence identity with the polypeptide of (i) or (ii).

2. An isolated polynucleotide encoding the polypeptide of claim 1.

3. The polypeptide of claim 1, wherein polypeptide blocks the interaction between Surfeit locus protein 4 (SURF4) and Sonic hedgehog (Shh).

4. The polypeptide of claim 1, wherein polypeptide binds to Shh.

5. A composition comprising the polypeptide of claim 1.

6. The composition of claim 5, further comprising a pharmaceutically acceptable carrier and/or excipient.

7. A method for inhibiting the Hh signaling pathway, the method comprising administering a composition comprising a large molecule that blocks the interaction between SURF4 and Shh or the polypeptide molecule of claim 1 to a subject.

8. The method of claim 7, wherein the polypeptide molecule blocks the interaction between SURF4 and Shh.

9. The method of claim 7, wherein the polypeptide molecule binds to Shh.

10. The method of claim 7, wherein the large molecule is a glycosaminoglycan.

11. The method of claim 10, wherein the glycosaminoglycan is heparin, heparin sulfate, heparin sulfate proteoglycans (HSPG), or chondroitin sulfate proteoglycans (CSPGs).

12. The method of claim 7, wherein the subject has a ligand-dependent cancer.

13. The method of claim 7, wherein the subject is a human.

14. The method of claim 7, wherein the composition further comprises a pharmaceutically acceptable carrier and/or excipient.

15. A method for inhibiting the Hh signaling pathway, the method comprising mutating in a subject:
i) amino acid residue E50, D53, D56, or any combination thereof of human SURF4;
ii) a nucleotide encoding amino acid residues E50, D53, D56, or any combination thereof of human SURF4;
iii) amino acid residue 32, 33, 34, 35, 36, 37, 38, of human Shh or any combination thereof; or
iv) a nucleotide encoding amino acid residue 32, 33, 34, 35, 36, 37, 38, or any combination thereof of human Shh.

16. The method of claim 15, wherein the mutations of i)-iv) inhibit the interaction of SURF4 and Shh in a subject.

17. The method of claim 15, wherein the subject has a ligand-dependent cancer.

18. The method of claim 15, wherein the subject is a human.


SEQUENCE LISTING (18 sequences)

SEQ ID NO: 1 - RNA, 20 nt, synthetic construct
  source 1..20: mol_type = other RNA
agtcgcgctgctcgctccac

SEQ ID NO: 2 - RNA, 19 nt, synthetic construct
  source 1..19: mol_type = other RNA
gcaggaacttcgtgcagta

SEQ ID NO: 3 - RNA, 19 nt, synthetic construct
  source 1..19: mol_type = other RNA
gcatccgtatgtggttcca

SEQ ID NO: 4 - RNA, 19 nt, synthetic construct
  source 1..19: mol_type = other RNA
ctggtagtgtggagcttca

SEQ ID NO: 5 - RNA, 19 nt, synthetic construct
  source 1..19: mol_type = other RNA
gcgtgcacctgtatttcta

SEQ ID NO: 6 - protein, 8 aa, synthetic construct
KRRHPKKC

SEQ ID NO: 7 - protein, 7 aa, synthetic construct
RRRHPKK

SEQ ID NO: 8 - protein, 7 aa, synthetic construct
KRRHPKK

SEQ ID NO: 9 - protein, 8 aa, synthetic construct
AAAHPAAC

SEQ ID NO: 10 - protein, 13 aa, synthetic construct
SEQRDYIDTTWNC

SEQ ID NO: 11 - protein, 11 aa, synthetic construct
VRRFRYPERPC

SEQ ID NO: 12 - protein, 12 aa, synthetic construct
SEQRDYIDTTWN

SEQ ID NO: 13 - DNA, 21 bp, synthetic construct
  source 1..21: mol_type = other DNA
aagaggaggcaccccaaaaag

SEQ ID NO: 14 - DNA, 39 bp, synthetic construct
  source 1..39: mol_type = other DNA
agcgagcagcgcgactacatcgacaccacctggaactgc

SEQ ID NO: 15 - protein, 269 aa, Homo sapiens
MGQNDLMGTAEDFADQFLRVTKQYLPHVARLCLISTFLEDGIRMWFQWSEQRDYIDTTWNCGYLLASSFVFLNLLGQLTGCVLVLSRNFVQYACFGLFGIIALQTIAYSILWDLKFLMRNLALGGGLLLLLAESRSEGKSMFAGVPTMRESSPKQYMQLGGRVLLVLMFMTLLHFDASFFSIVQNIVGTALMILVAIGFKTKLAALTLVVWLFAINVYFNAFWTIPVYKPMHDFLKYDFFQTMSVIGGLLLVVALGPGGVSMDEKKKEW

SEQ ID NO: 16 - DNA, 810 bp, Homo sapiens
  source 1..810: mol_type = other DNA
atgggccagaacgacctgatgggcacggccgaggacttcgccgaccagttcctccgtgtcacaaagcagtacctgccccacgtggcgcgcctctgtctgatcagcaccttcctggaggacggcatccgtatgtggttccagtggagcgagcagcgcgactacatcgacaccacctggaactgcggctacctgctggcctcgtccttcgtcttcctcaacttgctgggacagctgactggctgcgtcctggtgttgagcaggaacttcgtgcagtacgcctgcttcgggctctttggaatcatagctctgcagacgattgcctacagcattttatgggacttgaagtttttgatgaggaacctggccctgggaggaggcctgttgctgctcctagcagaatcccgttctgaagggaagagcatgtttgcgggcgtccccaccatgcgtgagagctcccccaaacagtacatgcagctcggaggcagggtcttgctggttctgatgttcatgaccctccttcactttgacgccagcttcttttctattgtccagaacatcgtgggcacagctctgatgattttagtggccattggttttaaaaccaagctggctgctttgactcttgttgtgtggctctttgccatcaacgtatatttcaacgccttctggaccattccagtctacaagcccatgcatgacttcctgaaatacgacttcttccagaccatgtcggtgattgggggcttgctcctggtggtggccctgggccctgggggtgtctccatggatgagaagaagaaggagtggtaa

SEQ ID NO: 17 - protein, 462 aa, Homo sapiens
MLLLARCLLLVLVSSLLVCSGLACGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLSPGDRVLAADDQGRLLYSDFLTFLDRDDGAKKVFYVIETREPRERLLLTAAHLLFVAPHNDSATGEPEASSGSGPPSGGALGPRALFASRVRPGQRVYVVAERDGDRRLLPAAVHSVTLSEEAAGAYAPLTAQGTILINRVLASCYAVIEEHSWAHRAFAPFRLAHALLAALAPARTDRGGDSGGGDRGGGGGRVALTAPGAADAPGAGATAGIHWYSQLLYQIGTWLLDSEALHPLGMAVKSS

SEQ ID NO: 18 - DNA, 1389 bp, Homo sapiens
  source 1..1389: mol_type = other DNA
atgctgctgctggcgagatgtctgctgctagtcctcgtctcctcgctgctggtatgctcgggactggcgtgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggctgcttcccgggctcggccacggtgcacctggagcagggcggcaccaagctggtgaaggacctgagccccggggaccgcgtgctggcggcggacgaccagggccggctgctctacagcgacttcctcactttcctggaccgcgacgacggcgccaagaaggtcttctacgtgatcgagacgcgggagccgcgcgagcgcctgctgctcaccgccgcgcacctgctctttgtggcgccgcacaacgactcggccaccggggagcccgaggcgtcctcgggctcggggccgccttccgggggcgcactggggcctcgggcgctgttcgccagccgcgtgcgcccgggccagcgcgtgtacgtggtggccgagcgtgacggggaccgccggctcctgcccgccgctgtgcacagcgtgaccctaagcgaggaggccgcgggcgcctacgcgccgctcacggcccagggcaccattctcatcaaccgggtgctggcctcgtgctacgcggtcatcgaggagcacagctgggcgcaccgggccttcgcgcccttccgcctggcgcacgcgctcctggctgcactggcgcccgcgcgcacggaccgcggcggggacagcggcggcggggaccgcgggggcggcggcggcagagtagccctaaccgctccaggtgctgccgacgctccgggtgcgggggccaccgcgggcatccactggtactcgcagctgctctaccaaataggcacctggctcctggacagcgaggccctgcacccgctgggcatggcggtcaagtccagctga